(19) United States
(12) Patent Application Publication
(10) Pub. No.: US 2014/0296248 A1
(43) Pub. Date: Oct. 2, 2014

(54) METHODS AND COMPOSITIONS FOR PREDICTING RESISTANCE TO ANTICANCER TREATMENT

(71) Applicants: Rene Bernards, Amsterdam (NL); Sidong Huang, Amsterdam (NL); Michael Holzel, Amsterdam (NL)

(72) Inventors: Rene Bernards, Amsterdam (NL); Sidong Huang, Amsterdam (NL); Michael Holzel, Amsterdam (NL)

(73) Assignee: Stichting het Nederlands Kanker Instiuut-Antoni van Leeuwenhoek ziekenhuis, Amsterdam (NL)

(21) Appl. No.: 14/009,448

(22) PCT Filed: Apr. 4, 2012
(86) PCT No.: PCT/US12/32202

§ 371 Date: May 8, 2014

Related U.S. Application Data
(60) Provisional application No. 61/471,601, filed on Apr. 4, 2011.
(60) Provisional application No. 61/472,165, filed on Apr. 5, 2011.
(60) Provisional application No. 61/610,349, filed on Mar. 13, 2012.

(51) Int. Cl. C12Q 1/68

(52) U.S. Cl. CPC C12Q 1/6886
USPC 514/252.18; 506/9; 435/6.11; 514/318

(57) ABSTRACT

The instant application provides methods and related compositions pertaining to the identification of resistance to anticancer treatment in a patient. In a particular embodiment, the invention provides biomarkers for the identification of resistance to anticancer treatment in a lung cancer patient, wherein a reduced expression of a MEDIATOR and/or SW1/SNF complex gene in the lung cancer cells of the patient indicates that the lung cancer cells in the patient may be resistant to treatment with a receptor tyrosine kinase inhibitor, such as gefitinib and/or erlotinib. In some embodiments, the invention relates to methods and related compositions for predicting resistance to anticancer treatment by detecting the expression levels of one or more TGF-beta pathway nucleic acids and/or proteins.

### CROSS-REFERENCE TO RELATED APPLICATIONS

This application claims the benefit of priority of U.S. Provisional Application Ser. No. 61/471,601 filed Apr. 4, 2011; U.S. Provisional Application Ser. No. 61/472,165, filed Apr. 5, 2011; and U.S. Provisional Application Ser. No. 61/610,349 filed Mar. 13, 2012, which are incorporated herein by reference in their entirety.

### FIELD OF THE INVENTION

The invention relates to the field of methods and related compositions for predicting resistance to anticancer treatment. In certain embodiments, the invention relates to the field of methods and related compositions for predicting resistance to anticancer treatment in a cancer patient by detecting a reduced expression level of a SWI/SNF complex and/or MEDIATOR complex and/or RAS-GAP gene and/or protein in one or more cancer cells of the patient. In other embodiments, the invention relates to the field of methods and related compositions for predicting resistance to anticancer treatment by detecting one or more inactivating mutations in a SWI/SNF complex and/or MEDIATOR complex and/or RAS-GAP gene. In some embodiments, the invention relates to the field of methods and related compositions for predicting resistance to anticancer treatment by detecting dysfunction and/or inactivity of one or more SWI/SNF complex and/or MEDIATOR complex and/or RAS-GAP proteins. In some embodiments, the invention relates to the field of methods and related compositions for predicting resistance to anticancer treatment by detecting the expression levels of one or more TGF-beta pathway nucleic acids and/or proteins.

### BACKGROUND OF THE INVENTION

Activation of signaling pathways in cancer is often the result of genomic alterations (mutations, translocations, copy number gains and/or losses) in key components of these pathways. Cancer cells often depend on the continued presence of the signals that emanate from these genomic alterations and sudden inhibition frequently results in death of the cancer cells, a phenomenon coined “oncogene addiction” (Sharma and Settleman, 2007). The presence of specific changes in the genomes of cancer cells can therefore have strong predictive value for responsiveness to therapies that target these mutations (Pao and Chmielecki).

Such drug response biomarkers are urgently needed for the rational selection of patients for these therapies, as their clinical benefit is often limited due to the fact that only a subset of patients responds. Considering the high cost of targeted therapeutics, response biomarkers are not only a clinical necessity, but also an economic requirement to keep the cost of cancer care in check by reducing the number of patients that receive expensive drugs without experiencing therapeutic benefit.

Lung cancer is a leading cause of cancer deaths worldwide and tobacco smoking remains the major risk factor. Genomic alterations of receptor tyrosine kinases are frequently found in non-small cell lung cancers, the predominant histological subtype, and are particularly enriched (˜40%) in non-smokers (Rudin et al., 2009). Lung cancers with activating mutations of the EGFR (epidermal growth factor receptor) respond well to treatment with EGFR inhibitors (gefitinib and erlotinib) in the clinic and constitute the largest subgroup of patients (˜10%-20%) tractable for an effective tyrosine kinase inhibitor therapy (Lynch et al., 2004; Maemondo et al.; Rosell et al., 2009; Sharma et al., 2007). Recently, EML4-ALK translocations were identified in ˜2%-5% of NSCLC providing a second promising molecular target for the treatment of NSCLC (Soda et al., 2007). The fusion of the N-terminal part of EML4 (echinoderm microtubule associated protein like 4) with the C-terminal kinase domain of ALK (anaplastic large cell lymphoma kinase) results in the stable dimerization and constitutive activation of the EML4-ALK fusion kinase. The dual tyrosine kinase inhibitor crizotinib potently inhibits ALK/MET and is currently evaluated in clinical trials. The first phase I study with crizotinib in EML4-ALK positive advanced NSCLC demonstrated remarkable activity (Kwak et al.).

Despite these encouraging clinical results, lung cancers with EGFR mutations or EML4-ALK translocations do not respond equally well to these inhibitors (primary resistance) and all tumors develop resistance (acquired resistance) under prolonged treatment (Jackman et al.). Several acquired resistance mechanisms were identified in pre-clinical studies and also confirmed in specimens from relapsed patients that initially responded well to EGFR or ALK inhibitor treatment. Second site mutations of the EGFR (EGFR^{T790M}) and MET amplifications account for ˜50% of the cases of acquired resistance to EGFR inhibitors (Engelman et al., 2007; Hammerman et al., 2009; Kobayashi et al., 2005). The EGFR^{T790M} gatekeeper mutation prevents binding of the inhibitors to the kinase domain, but preserves the activity of the kinase. The frequency of EML4-ALK second site mutations in relapsed tumors is currently unknown and was only found in a single case so far (Choi et al.).

Nevertheless, in a large number of cases the mechanism of resistance to EGFR or ALK inhibitors remains unknown and in particular the determinants of primary resistance are obscure. Understanding the relevant genes and signaling pathways that contribute to resistance of NSCLC cells to tyrosine kinase inhibitors might not only provide drug response markers to stratify treatment options, but might also delineate new therapeutic strategies to overcome the drug resistance.

Citation or identification of any document in this application is not an admission that such document is available as prior art to the present invention.

### SUMMARY OF THE INVENTION

In certain embodiments, the invention provides a method of evaluating and/or predicting resistance to anticancer treatment in a patient in need thereof, comprising (a) measuring expression levels of one or more SWI/SNF complex and/or MEDIATOR complex nucleic acid and/or proteins in the patient; and (b) comparing the expression levels of the one or more SWI/SNF complex and/or MEDIATOR complex nucleic acid and/or proteins in (a) with the expression levels of one or more reference SWI/SNF complex and/or MEDIATOR complex nucleic acid and/or proteins, wherein the one or more reference SWI/SNF complex and/or MEDIATOR complex nucleic acid and/or proteins are from a control sample, wherein a reduction in the expression of the one or more SWI/SNF complex and/or MEDIATOR complex nucleic acid and/or proteins in comparison to the one or more reference SWI/SNF complex and/or MEDIATOR complex nucleic acid and/or proteins is indicative of resistance to anticancer treatment in the patient.

In other embodiments, the invention provides a method of evaluating and/or predicting resistance to anticancer treatment in a patient in need thereof, comprising (a) isolating nucleic acid from the patient, wherein the nucleic acid comprises one or more SWI/SNF complex and/or MEDIATOR complex DNA and/or RNA; and (b) analyzing the nucleic acid of (a) for the presence of one or more inactivating mutations in the SWI/SNF complex and/or MEDIATOR complex DNA and/or RNA, wherein the presence of one or more inactivating mutations in the one or more SWI/SNF complex and/or MEDIATOR complex DNA and/or RNA analyzed in (b) is indicative of resistance to anticancer treatment in the patient.

In some embodiments, the invention relates to a method of evaluating and/or predicting resistance to anticancer treatment in a patient in need thereof, comprising (a) isolating protein from the patient, wherein the protein comprises one or more SWI/SNF complex and/or MEDIATOR complex proteins (b) analyzing the activity of the one or more SWI/SNF complex and/or MEDIATOR complex proteins in (a); and (c) comparing the activity of the one or more SWI/SNF complex and/or MEDIATOR complex proteins in (b) with the activity of one or more reference SWI/SNF complex and/or MEDIATOR complex proteins, wherein a difference in activity of the one or more SWI/SNF complex and/or MEDIATOR complex proteins from (b) in comparison to the one or more SWI/SNF complex and/or MEDIATOR complex reference proteins in (c) is indicative of resistance to anticancer treatment in the patient.

In certain embodiments, the expression levels of one or more SWI/SNF complex nucleic acids (e.g., DNA, RNA) and/or proteins are measured.

In certain embodiments, the expression levels of one or more MEDIATOR complex nucleic acids (e.g., DNA, RNA) and/or proteins are measured.

In some embodiments, the invention provides a method of evaluating and/or predicting resistance to anticancer treatment in a patient in need thereof, comprising (a) measuring expression levels of one or more RAS-GAP nucleic acid and/or proteins in the patient; and (b) comparing the expression levels of the one or more RAS-GAP nucleic acid and/or proteins in (a) with the expression levels of one or more reference RAS-GAP nucleic acid and/or proteins, wherein the one or more reference RAS-GAP nucleic acid and/or proteins are from a control sample, wherein a reduction in the expression of the one or more RAS-GAP nucleic acid and/or proteins in comparison to the one or more reference RAS-GAP nucleic acid and/or proteins is indicative of resistance to anticancer treatment in the patient.

In other embodiments, the invention provides a method of evaluating and/or predicting resistance to anticancer treatment in a patient in need thereof, comprising (a) isolating nucleic acid from the patient, wherein the nucleic acid comprises one or more RAS-GAP DNA and/or RNA; and (b) analyzing the nucleic acid of (a) for the presence of one or more inactivating mutations in the RAS-GAP DNA and/or RNA, wherein the presence of one or more inactivating mutations in the one or more RAS-GAP DNA and/or RNA analyzed in (b) is indicative of resistance to anticancer treatment in the patient.

In yet other embodiments, the invention provides a method of evaluating and/or predicting resistance to anticancer treatment in a patient in need thereof, comprising (a) isolating protein from the patient, wherein the protein comprises one or more RAS-GAP proteins; (b) analyzing the activity of the one or more RAS-GAP proteins in (a); and (c) comparing the activity of the one or more RAS-GAP proteins in (b) with the activity of one or more reference RAS-GAP proteins, wherein a difference in activity of the one or more RAS-GAP proteins from (b) in comparison to the one or more RAS-GAP reference proteins in (c) is indicative of resistance to anticancer treatment in the patient.

In some embodiments the expression levels of one or more RAS-GAP nucleic acids (e.g., DNA, RNA) are measured. In other embodiments, the expression levels of one or more RAS-GAP proteins are measured.

In some embodiments of the methods described herein for evaluating and/or predicting resistance to anticancer treatment in a patient in need thereof, the patient has lung cancer (e.g., non-small-cell lung cancer), breast cancer, ovarian cancer, lung cancer, head and neck cancer, bladder cancer, colorectal cancer, cervical cancer, mesothelioma, solid tumors, renal cell carcinoma, stomach cancer, sarcoma, prostate cancer, melanoma, thyroid cancer, brain cancer, adenocarcinoma, glioma, glioblastoma, esophageal cancer, neuroblastoma, and/or lymphoma.

In some embodiments, the resistance to anticancer treatment is resistance to treatment with a receptor tyrosine kinase inhibitor. Examples of receptor tyrosine kinase inhibitors include gefitinib, erlotinib, EKB-569, lapatinib, CI-1033, cetuximab, panitumumab, PKI-166, AEE788, sunitinib, sorafenib, dasatinib, nilotinib, pazopanib, vandetaniv, cediranib, afatinib, motesanib, CUDC-101, imatinib mesylate, crizotinib, ASP-3026, LDK378, AF802, and CEP37440.

In some embodiments, the resistance to anticancer treatment is resistance to treatment with an inhibitor of ERK activation. In certain embodiments, the inhibitor of ERK activation inhibits a cellular protein that interacts directly with ERK. In other embodiments, the inhibitor of ERK activation inhibits a cellular protein that interacts indirectly with ERK. In yet other embodiments, the inhibitor of ERK activation is a receptor tyrosine kinase inhibitor.

Examples of SWI/SNF complex nucleic acids and/or proteins include ARID1A, ARID1B, ARID2, SMARCA2, SMARCA4, PBRM1, SMARCC2, SMARCC1, SMARCD1, SMARCD2, SMARCD3, SMARCE1, ACTL6A, ACTL6B, and SMARCB1.

Examples of MEDIATOR complex nucleic acids and/or proteins include MED22, MED11, MED17, MED20, MED30, MED19, MED18, MED8, MED6, MED28, MED9, MED21, MED4, MED7, MED31, MED10, MED1, MED26, MED2, MED3, MED25, MED23, MED5, MED14, MED16, MED15, CycC, CDK8, MED13, MED12, MED12L, and MED13L.

Examples of RAS-GAP nucleic acids and/or proteins include DAB2IP, NF1, and RASAL3.

In some embodiments, analyzing nucleic acid comprises sequencing the nucleic acid. In other embodiments, analyzing nucleic acid comprises subjecting the nucleic acid to MLPA. In yet other embodiments, analyzing nucleic acid comprises subjecting the nucleic acid to CGH. In certain embodiments, analyzing nucleic acid comprises subjecting the nucleic acid to FISH.

In certain embodiments, an inactivating mutation is selected from the group consisting of: point mutations, translocations, amplifications, deletions, and hypomorphic mutations.

In certain embodiments, nucleic acid in a method of the invention comprises one or more SWI/SNF complex genes. In other embodiments, the nucleic acid comprises one or more MEDIATOR complex genes. In yet other embodiments, the nucleic acid comprises one or more RAS-GAP genes.

In certain embodiments, one or more SWI/SNF complex and/or MEDIATOR complex proteins analyzed are inactive. In further embodiments, the one or more SWI/SNF complex and/or MEDIATOR complex proteins are inactive due to one or more posttranslational modifications. In some embodiments, one or more RAS-GAP proteins analyzed are inactive. In further embodiments, the one or more RAS-GAP proteins are inactive due to one or more posttranslational modifications

In some embodiments, the invention relates to a microarray comprising a plurality of polynucleotide probes each complementary and hybridizable to a sequence in a different gene that is a SWI/SNF complex gene that is a marker for resistance to anticancer treatment in a patient that has cancer.

In other embodiments, the invention relates to a microarray comprising a plurality of polynucleotide probes each complementary and hybridizable to a sequence in a different gene that is a MEDIATOR complex gene that is a marker for resistance to anticancer treatment in a patient that has cancer.

In some embodiments, the invention relates to a microarray comprising a plurality of polynucleotide probes each complementary and hybridizable to a sequence in a different gene that is a SWI/SNF complex and/or MEDIATOR complex gene that is a marker for resistance to anticancer treatment in a patient that has cancer.

In other embodiments, the invention relates to a microarray comprising a plurality of polynucleotide probes each complementary and hybridizable to a sequence in a different gene that is a RAS-GAP gene that is a marker for resistance to anticancer treatment in a patient that has cancer.

In certain embodiments, a microarray of the invention comprises a plurality of probes, wherein the plurality of probes is at least 70%, at least 80%, at least 90%, at least 95%, or at least 98% of the probes on the microarray.

In certain embodiments, in a microarray of the invention, the SWI/SNF complex gene that is a marker for resistance to anticancer treatment is selected from the group consisting of ARID1A, ARID1B, ARID2, SMARCA2, SMARCA4, PBRM1, SMARCC2, SMARCC1, SMARCD1, SMARCD2, SMARCD3, SMARCE1, ACTL6A, ACTL6B, and SMARCB1.

In other embodiments, in a microarray of the invention, the MEDIATOR complex gene that is a marker for resistance to anticancer treatment is selected from the group consisting of MED22, MED11, MED17, MED20, MED30, MED19, MED18, MED8, MED6, MED28, MED9, MED21, MED4, MED7, MED31, MED10, MED1, MED26, MED2, MED3, MED25, MED23, MED5, MED14, MED16, MED15, CycC, CDK8, MED13, MED12, MED13L, and MED12L.

In still other embodiments, in a microarray of the invention, the RAS-GAP gene is selected from the group consisting of: DAB2IP, NF1, and RASAL3.

In some embodiments, the invention relates to a kit, comprising at least one pair of primers specific for a SWI/SNF complex gene that is a marker for resistance to anticancer treatment in a patient that has cancer, at least one reagent for amplification of the SWI/SNF complex gene, and instructions for use.

In other embodiments, the invention relates to a kit, comprising at least one pair of primers specific for a MEDIATOR complex gene that is a marker for resistance to anticancer treatment in a patient that has cancer, at least one reagent for amplification of the MEDIATOR complex gene, and instructions for use.

In some embodiments, the invention relates to a kit, comprising at least one pair of primers specific for a SWI/SNF complex and/or a MEDIATOR complex gene that is a marker for resistance to anticancer treatment in a patient that has cancer, at least one reagent for amplification of the SWI/SNF complex and/or MEDIATOR complex gene, and instructions for use.

In other embodiments, the invention relates to a kit, comprising at least one pair of primers specific for a RAS-GAP gene that is a marker for resistance to anticancer treatment in a patient that has cancer, at least one reagent for amplification of the RAS-GAP gene, and instructions for use.

In certain embodiments, in a kit of the invention, the primers are specific for a SWI/SNF complex gene selected from the group consisting of ARID1A, ARID1B, ARID2, SMARCA2, SMARCA4, PBRM1, SMARCC2, SMARCC1, SMARCD1, SMARCD2, SMARCD3, SMARCE1, ACTL6A, ACTL6B, and SMARCB1.

In certain embodiments, in a kit of the invention, the primers are specific for a MEDIATOR complex gene selected from the group consisting of MED22, MED11, MED17, MED20, MED30, MED19, MED18, MED8, MED6, MED28, MED9, MED21, MED4, MED7, MED31, MED10, MED1, MED26, MED2, MED3, MED25, MED23, MED5, MED14, MED16, MED15, CycC, CDK8, MED13, MED12, MED13L, and MED12L.

In certain embodiments, in a kit of the invention, the primers are specific for a RAS-GAP gene selected from the group, consisting of: DAB2IP, NF1, and RASAL3.

In certain embodiments, in a kit of the invention, the marker for resistance to anticancer treatment is a marker for resistance to a receptor tyrosine kinase inhibitor.

In certain embodiments, in a kit of the invention, the marker for resistance to anticancer treatment is a marker for resistance to an inhibitor of ERK activation. In some embodiments, the inhibitor of ERK activation inhibits a cellular protein that interacts directly with ERK. In some embodiments, the inhibitor of ERK activation inhibits a cellular protein that interacts indirectly with ERK. In other embodiments, the inhibitor of ERK activation is a receptor tyrosine kinase inhibitor.

In certain embodiments, the kit is a PCR kit. In other embodiments, the kit is an MLPA kit. In yet other embodiments, the kit is an RT-MLPA kit.

In some embodiments, the level of expression of one or more SWI/SNF complex and/or MEDIATOR complex and/or RAS-GAP genes in a method of the invention is measured by determination of their level of transcription, using a DNA array. In other embodiments, the level of expression of one or more SWI/SNF complex and/or MEDIATOR complex and/or RAS-GAP genes is measured by determination of their level of transcription, using quantitative RT-PCR.

In some embodiments the level of expression of one or more SWI/SNF complex and/or MEDIATOR complex and/or RAS-GAP genes in a method of the invention is measured in a tumor sample from the patient. In certain further embodiments, the tumor sample is a lung tumor sample.

In some embodiments, the resistance to anticancer treatment is resistance to treatment with a B-RAF inhibitor. Examples of B-RAF inhibitors include CEP-32496, vemurafenib, GSK-2118436, ARQ-736, RG-7256, XL-281, DCC-2036, GDC-0879, AZ628, and antibody fragment EphB4/Raf inhibitors.

In some embodiments, resistance to anticancer treatment is resistance to treatment with a MEK inhibitor. Examples of MEK inhibitors include CKI-27, RO-4987655, RO-5126766, PD-0325901, WX-554, AZD-8330, G-573, RG-7167, SF-2626, GDC-0623, RO-5068760, and AD-GL0001.

In certain embodiments, in a kit of the invention, the marker for resistance to anticancer treatment is a marker for resistance to treatment with a B-RAF inhibitor. In other embodiments, the marker for resistance to anticancer-treatment is a marker for resistance to treatment with a MEK inhibitor.

In certain embodiments, in the methods of the invention, the expression levels of SWI/SNF and/or MEDIATOR complex or RAS-GAP nucleic acid and/or proteins are measured in one or more cancer cells of the patient. In some embodiments, nucleic acid is isolated from one or more cancer cells of the patient. In other embodiments, protein is isolated from one or more cancer cells of the patient.

In certain embodiments, in a method of the invention, resistance to anticancer treatment in one or more cancer cells in a patient is primary resistance to anticancer treatment. In other embodiments, the resistance is secondary resistance to anticancer treatment.

In certain embodiments, the instant application relates to a method of treating resistance to one or more inhibitors of ERK activation in a patient in need thereof, comprising administering to the patient at least one inhibitor of the TGF-beta pathway in combination with the one or more inhibitors of ERK activation. In some embodiments, the inhibitor of ERK activation is selected from the group consisting of direct and indirect inhibitors of ERK activation. In certain embodiments, the direct inhibitor of ERK activation is a MEK inhibitor. In certain embodiments, the indirect inhibitor of ERK activation is selected from the group consisting of RTK inhibitors, RAS inhibitors, and B-RAF inhibitors.

In some embodiments, the resistance to one or more inhibitors of ERK activation is primary resistance. In other embodiments, the resistance to one or more inhibitors of ERK activation is secondary resistance. In yet other embodiments, the resistance to one or more inhibitors of ERK activation is evaluated and/or predicted according to a method as disclosed herein.

In other embodiments, the instant application relates to a method of evaluating and/or predicting resistance to anticancer treatment in a patient in need thereof, comprising (a) measuring expression levels of one or more TGFβ pathway nucleic acid and/or proteins in the patient; and (b) comparing the expression levels of the one or more TGFβ pathway nucleic acid and/or proteins in (a) with the expression levels of one or more reference TGFβ pathway nucleic acid and/or proteins, wherein the one or more reference TGFβ pathway nucleic acid and/or proteins are from a control sample, wherein an increase in the expression of the one or more TGFβ pathway nucleic acid and/or proteins in comparison to the one or more reference TGFβ pathway nucleic acid and/or proteins is indicative of resistance to anticancer treatment in the patient.

In yet other embodiments, the instant application relates to a method of evaluating and/or predicting resistance to anticancer treatment in a patient in need thereof, comprising (a) isolating nucleic acid from the patient, wherein the nucleic acid comprises one or more TGFβ pathway DNA and/or RNA; and (b) analyzing the nucleic acid of (a) for the presence of one or more activating mutations in the TGFβ pathway complex DNA and/or RNA, wherein the presence of one or more activating mutations in the one or more TGFβ pathway DNA and/or RNA analyzed in (b) is indicative of resistance to anticancer treatment in the patient.

In some embodiments, the instant application relates to a method of evaluating and/or predicting resistance to anticancer treatment in a patient in need thereof, comprising (a) isolating protein from the patient, wherein the protein comprises one or more TGFβ pathway proteins; (b) analyzing the activity of the one or more TGFβ pathway proteins in (a); and (c) comparing the activity of the one or more TGFβ pathway proteins in (b) with the activity of one or more reference TGFβ pathway proteins, wherein a difference in activity of the one or more TGFβ pathway proteins from (b) in comparison to the one or more TGFβ pathway reference proteins in (c) is indicative of resistance to anticancer treatment in the patient.

In certain embodiments, the instant application relates to a method of treating cancer in a patient in need thereof, comprising administering to the patient an inhibitor of ERK activation in combination with an inhibitor of TGFβ pathway activation. In certain further embodiments, the cancer is selected from the group consisting of: liver cancer, lung cancer, breast cancer, ovarian cancer, head and neck cancer, bladder cancer, colorectal cancer, cervical cancer, mesothelioma, solid tumors, renal cell carcinoma, stomach cancer, sarcoma, prostate cancer, melanoma, thyroid cancer, brain cancer, adenocarcinoma, glioma, glioblastoma, esophageal cancer, neuroblastoma, subependymal giant cell astrocytoma, endometrial cancer, a hematological cancer, and lymphoma.

In certain embodiments, the inhibitor of ERK activation is selected from the group consisting of: RTK inhibitors, RAS inhibitors, B-RAF inhibitors, and MEK inhibitors. In a particular embodiment, the inhibitor of ERK activation is a MET inhibitor.

In certain embodiments, the expression levels are measured of one or more of TGFβ pathway nucleic acid that is a TGFβ pathway target gene selected from the group consisting of: ALOX5AP, COL5A, TAGLN, ANGPTL4, LGALS1, IL11, LBH, and COL4A1.

In some embodiments, the inhibitor of TGFβ pathway activation is LY2157299. In certain embodiments, the inhibitor of TGFβ pathway activation inhibits MED12/TGFβ binding.

In some embodiments, inhibitor of ERK activation is crizotinib or gefitinib. In certain embodiments, the inhibitor of ERK activation inhibits MED12/TGFβ binding.

In some embodiments, the instant application relates to a method of identifying an inhibitor of ERK activation, comprising: measuring MED12/TGFβ binding in the presence and absence of a test compound, wherein a reduction in the amount of MED12/TGFβ binding in the presence of the test compound in comparison to the absence of the test compound indicates an inhibitor of ERK activation has been identified.

In other embodiments, the instant application relates to a method of identifying an inhibitor of TGFβ pathway activation, comprising: measuring MED12/TGFβ binding in the presence and absence of a test compound, wherein a reduction in the amount of MED12/TGFβ binding in the presence of the test compound in comparison to the absence of the test compound indicates an inhibitor of TGFβ pathway activation has been identified.

In yet other embodiments, the instant application relates to a method of evaluating and/or predicting resistance to anticancer treatment in a patient in need thereof, comprising: (a) measuring expression levels of one or more MED12 nucleic acid and/or proteins in the patient; (b) measuring one or more markers of an EMT-like phenotype; and (c) comparing the expression levels of the one or more MED12 nucleic acid and/or proteins in (a) with the expression levels of one or more reference MED12 nucleic acid and/or proteins, wherein a reduction in the expression of the one or more MED12 nucleic acid and/or proteins in comparison to the one or more reference MED12 nucleic acid and/or proteins in (c) and wherein one or more markers are measured of an EMT-like phenotype in (b) is indicative of resistance to anticancer treatment in the patient.

In some embodiments, the nucleic acid in (a) is isolated from one or more cancer cells from the patient. In other embodiments, the protein in (a) is isolated from one or more cancer cells from the patient. In certain embodiments, the one or more markers of an EMT-like phenotype are measured in one or more cancer cells from the patient. In certain further embodiments, the cancer is selected from the group consisting of: liver cancer, lung cancer, breast cancer, ovarian cancer, head and neck cancer, bladder cancer, colorectal cancer, cervical cancer, mesothelioma, solid tumors, renal cell carcinoma, stomach cancer, sarcoma, prostate cancer, melanoma, thyroid cancer, brain cancer, adenocarcinoma, glioma, glioblastoma, esophageal cancer, neuroblastoma, subependymal giant cell astrocytoma, endometrial cancer, a hematological cancer, and lymphoma. In a particular embodiment, the cancer is colorectal cancer.

In certain embodiments, the resistance to anticancer treatment is resistance to treatment with a MEK inhibitor. In further embodiments, the MEK inhibitor is selected from the group consisting of: CKI-27, RO-4987655, RO-5126766, PD-0325901, WX-554, AZD-8330, G-573, RG-7167, SF-2626, GDC-0623, RO-5068760, and AD-GL0001.

In some embodiments, the resistance to anticancer treatment is resistance to treatment with a B-RAF inhibitor. In certain further embodiments, the B-RAF inhibitor is selected from the group consisting of: CEP-32496, vemurafenib, GSK-2118436, ARQ-736, RG-7256, XL-281, DCC-2036, GDC-0879, AZ628, and antibody fragment EphB4/Raf inhibitors.

In some embodiments, the one or more markers of an EMT-like phenotype are selected from mesenchymal markers. In certain embodiments, the one or more mesenchymal markers are selected from vimentin and N-cadherin.

In other embodiments, the instant application relates to a method of evaluating and/or predicting resistance to anticancer treatment in a patient in need thereof, comprising: (a) measuring expression levels of one or more MED12KD signature nucleic acid and/or proteins in one or more cancer cells of the patient; and (b) comparing the expression levels of the one or more MED12KD signature nucleic acid and/or proteins in (a) with the expression levels of one or more positive reference MED12KD signature nucleic acid and/or proteins, wherein if expression of the one or more MED12KD signature nucleic acid and/or proteins in (a) is similar to the one or more positive reference MED12KD signature nucleic acid and/or proteins, then resistance to anticancer treatment is indicated in the patient. In certain embodiments, the expression of the one or more MED12KD signature nucleic acid and/or proteins in (a) is about 2-fold, about 3-fold, about 4-fold, about 5-fold, about 6-fold, about 7-fold, about 8-fold, about 9-fold, or about 10-fold greater or lesser than the one or more positive reference MED12KD signature nucleic acid and/or proteins. In other embodiments, the expression of the one or more MED12KD signature nucleic acid and/or proteins in (a) is about the same as the one or more positive reference MED12KD signature nucleic acid and/or proteins.

In yet other embodiments, the instant application relates to a method of evaluating and/or predicting resistance to anticancer treatment in a patient in need thereof, comprising: (a) measuring expression levels of one or more MED12KD signature nucleic acid and/or proteins in one or more cancer cells of the patient; and (b) comparing the expression levels of the one or more MED12KD signature nucleic acid and/or proteins in (a) with the expression levels of one or more negative reference MED12KD signature nucleic acid and/or proteins, wherein if expression of the one or more MED12KD signature nucleic acid and/or proteins in (a) is greater or lesser than the expression of the one or more negative reference MED12KD signature nucleic acid and/or proteins, then resistance to anticancer treatment is indicated in the patient. In some embodiments, the one or more cancer cells of the patient in (a) are from cancer cells of the patient after the anticancer treatment, and wherein the negative reference MED12KD signature nucleic acid and/or proteins are from one or more cancerous cells of the patient prior to the anticancer treatment. In certain embodiments, the expression of the one or more MED12KD signature nucleic acid and/or proteins in (a) is greater than or equal to about 1.2 fold higher or lower than the expression of the one or more negative reference. MED12KD signature nucleic acid and/or proteins.

In some embodiments, the one or more cancer cells of the patient in (a) are from one or more cancer cells of the patient prior to the anticancer treatment. In other embodiments, the one or more cancer cells of the patient in (a) are from one or more cancer cells of the patient after the anticancer treatment.

In certain embodiments, the negative reference MED12KD signature nucleic acid and/or proteins are from one or more non-cancerous cells of the patient. In some embodiments, the negative reference MED12KD signature nucleic acid and/or proteins are from one or more cells known to be sensitive to the anticancer treatment. In certain embodiments, the negative reference MED12KD signature nucleic acid and/or proteins is the average expression of the MED12KD signature nucleic acid and/or proteins in one or more tumor or cell line samples known to be sensitive to the anticancer treatment.

In some embodiments, the one or more MED12^{KD} signature nucleic acids are upregulated nucleic acids. In certain embodiments, the upregulated nucleic acids are selected from the upregulated nucleic acids presented in FIG. 37. In certain embodiments, the upregulated nucleic acids are selected from the upregulated nucleic acids presented in FIG. 40. In certain embodiments, the upregulated nucleic acids are selected from the upregulated nucleic acids presented in FIG. 39.

In other embodiments, the one or more MED12^{KD} signature nucleic acids are downregulated nucleic acids. In certain embodiments, the downregulated nucleic acids are selected from the downregulated nucleic acids presented in FIG. 37. In certain embodiments, the downregulated nucleic acids are selected from the downregulated nucleic acids presented in FIG. 40. In certain embodiments, the downregulated nucleic acids are selected from the downregulated nucleic acids presented in FIG. 39.

In some embodiments, the resistance to anticancer treatment is resistance to treatment with a MEK inhibitor. In certain embodiments, the MEK inhibitor is selected from the group consisting of: CKI-27, RO-4987655, RO-5126766, PD-0325901, WX-554, AZD-8330, G-573, RG-7167, SF-2626, GDC-0623, RO-5068760, and AD-GL0001.

In some embodiments, the resistance to anticancer treatment is resistance to treatment with a B-RAF inhibitor. In certain embodiments, the B-RAF inhibitor is selected from the group consisting of: CEP-32496, vemurafenib, GSK-2118436, ARQ-736, RG-7256, XL-281, DCC-2036, GDC-0879, AZ628, and antibody fragment EphB4/Raf inhibitors.

In certain embodiments, the cancer is selected from the group consisting of: liver cancer, lung cancer, breast cancer, ovarian cancer, head and neck cancer, bladder cancer, colorectal cancer, cervical cancer, mesothelioma, solid tumors, renal cell carcinoma, stomach cancer, sarcoma, prostate cancer, melanoma, thyroid cancer, brain cancer, adenocarcinoma, glioma, glioblastoma, esophageal cancer, neuroblastoma, subependymal giant cell astrocytoma, endometrial cancer, a hematological cancer, and lymphoma.

In some embodiments, the instant application relates to a method of evaluating and/or predicting of resistance to anticancer treatment in a patient in need thereof, comprising: measuring expression levels of one or more MED12KD signature nucleic acid and/or proteins in one or more cancer cells of the patient; and comparing the expression levels of the one or more MED12KD signature nucleic acid and/or proteins in (a) with the expression levels of (i) one or more MED12KD signature nucleic acid and/or proteins from cells known to be resistant to said anticancer treatment AND (ii) one or more MED12KD signature nucleic acid and/or proteins from cells known to be sensitive to said anticancer treatment, whereby the cancer cells of the patient are considered to be resistant if the difference in expression levels between the cells in (a) and the cells in (i) is smaller than the difference in expression levels between the cells in (a) and the cells in (ii).

In other embodiments, the instant application relates to a method of evaluating and/or predicting of resistance to anticancer treatment in a patient in need thereof, comprising measuring expression levels of one or more MED12KD signature nucleic acid and/or proteins in one or more cancer cells of the patient; and comparing the expression levels of the one or more MED12KD signature nucleic acid and/or proteins in (a) with the expression levels of (i) one or more MED12KD signature nucleic acid and/or proteins from cells known to be resistant to said anticancer treatment AND (ii) one or more MED12KD signature nucleic acid and/or proteins from cells known to be sensitive to said anticancer treatment, whereby the cancer cells of the patient are considered to be sensitive if the difference in expression levels between the cells in (a) and the cells in (i) is greater than the difference in expression levels between the cells in (a) and the cells in (ii).

In yet other embodiments, the present application relates to a method of evaluating and/or predicting of resistance to anticancer treatment in a patient in need thereof, comprising measuring expression levels of one or more MED12KD signature nucleic acid and/or proteins in one or more cancer cells of the patient; and comparing the expression levels of the one or more MED12KD signature nucleic acid and/or proteins in (a) with the average expression levels of (i) one or more MED12KD signature nucleic acid and/or proteins taken from two or more cell samples, whereby the cancer cells of the patient are considered to be resistant if the difference in expression levels of the one or more MED12KD signature nucleic acid and/or proteins between the cells in (a) and the average expression levels of the one or more MED12KD signature nucleic acid and/or proteins in (i) is greater than a factor 1.2.

These and other embodiments are disclosed or are obvious from and encompassed by, the following Detailed Description.

### BRIEF DESCRIPTION OF THE DRAWINGS

FIG. 1 depicts the results of a genome-wide RNAi screen that identifies MED12, ARID1A and SMARCE1 as critical determinants of drug sensitivity to ALK inhibitors in EML4-ALK mutant NSCLC cells. (A) Schematic outline of the ALK inhibitor resistance barcode screen performed in H3122 cells. Human shRNA library polyclonal virus was produced to infect H3122 cells, which were then left untreated (control) or treated with 5 nM NVP-TAE684. After 4 weeks of selection, shRNA inserts from both populations were recovered, labeled and hybridized to DNA. (B) Analysis of the relative abundance of the recovered shRNA cassettes from ALK inhibitor barcode experiment. Averaged data from three independent experiments were normalized and 2 log transformed. Among the 49 top shRNA candidates (M>1.5 and A>7), two independent shMED12, one shARID1A and one shSMARCE1 vectors were identified. (C) Individual shRNAs from the library targeting MED12, ARID1A and SMARCE1 confer resistance to ALK inhibitors. H3122 cells expressing the empty vector pRS, control shGFP, shMED12#1, shMED12#2, shARID1A or shSMARCE1, were left untreated for 2 weeks or treated with 300 nM Crizotinib or 2.5 nM NVP-TAE684 for 4 weeks, after which the cells were fixed, stained and photographed.

FIG. 2. A genome-wide RNAi screen identifies MED12 as a critical determinant of drug response to tyrosine kinase inhibitors in NSCLCs

(A) Schematic outline of the crizotinib resistance barcode screen performed in H3122 cells. NKI human shRNA library polyclonal virus was produced to infect H3122 cells, which were then left untreated (control) or treated with 300 nM crizotinib for 14 or 28 days, respectively. After selection, shRNA inserts from both populations were recovered, labeled and hybridized to DNA oligonucleotide barcode arrays. (B) Analysis of the relative abundance of the recovered shRNA cassettes from crizotinib barcode experiment. Averaged data from three independent experiments were normalized and 2 log transformed. Among the 43 top shRNA candidates (M>2 and A>7), two independent shMED12 vectors (in light gray at end of arrow points) were identified. (F to H) Suppression of MED12 also confers to EGFR inhibitors. F) Colony formation assay of PC9 cells expressing pLKO control or independent lentiviral shMED12 vectors (#4 and #5) were cultured in 50 nM gefitinib or 50 nM erlotinib. The cells were fixed, stained and photographed after 10 (untreated) or 28 days (treated). G) The level of knockdown of MED12 by each of the shRNAs was measured by examining the MED12 mRNA levels by qRT-PCR. Error bars denote SD. H) The level of knockdown of MED12 protein was measured by western blotting.

FIG. 3 depicts that suppression of MED12 confers drug resistance to ALK inhibitors in EML4-ALK mutant NSCLC cells. (A) Validation of independent retroviral shRNAs (in pRS vector) targeting MED12 in H3122 cells. The functional phenotypes of non-overlapping shMED12 vectors are indicated by the colony formation assay in 300 nM Crizotinib or 2.5 nM NVP-TAE684. The cells were fixed, stained and photographed after 2 weeks (untreated) or 4 weeks (ALK:inhibitors treatment). (B and C) The knockdown ability of each of the shRNAs was measured by examining the MED12 mRNA levels by qRT-PCR (B) and the MED12 protein levels by western blotting (C). Error bars denote standard deviation (SD). (D) Validation of independent lentiviral shRNAs (in pLKO vector) targeting MED12. The functional phenotypes of non-overlapping shMED12 vectors are indicated by the colony formation assay in 300 nM Crizotinib or 2.5 nM NVP-TAE684. The cells were fixed, stained and photographed after 2 weeks (untreated) or 4 weeks (ALK inhibitors treatment). (E and F) The knockdown ability of each of the shRNAs was measured by examining the MED12 mRNA levels by qRT-PCR (B) and the MED12 protein levels by western blotting. Error bars denote standard deviation (SD).

FIG. 4 shows that restoration of Med12 reverses the resistance to ALK inhibitors driven by MED12 knockdown in EML4-ALK mutant NSCLC cells. (A) Ectopic expression of mouse Med12 re-sensitizes the MED12 knockdown cells to ALK inhibitors. H3122 cells expressing pLKO control or shMED12 vectors were retrovirally infected with viruses containing pMX or pMX-Med12, and were grown in the absence or presence of 300 nM Crizotinib or 2.5 nM NVP-TAE684. Cells were then fixed, stained and photographed after 2 weeks (untreated) or 4 weeks (ALK inhibitors treatment). (B) The MED12/Med12 protein levels in H3122 cells (untreated) described in FIG. 4A. (C and D) The endogenous MED12 mRNA (C) and the exogenous Med12 mRNA were measured by qRT-PCR.

FIG. 5 shows that suppression of ARID1A or SMARCE1 confers drug resistance to ALK inhibitors in EML4-ALK mutant NSCLC cells. (A) Validation of independent retroviral shRNAs targeting ARID1A or SMARCE1 in H3122 cells. The functional phenotypes of non-overlapping shARID1A and shSMARCE1 vectors are indicated by the colony formation assay in 300 nM Crizotinib or 2.5 nM NVP-TAE684. The cells were fixed, stained and photographed after 2 weeks (untreated) or 4 weeks (ALK inhibitors treatment). (B and C) The knockdown ability of each of the shRNAs was measured by examining the ARID1A mRNA levels by qRT-PCR (B) and the ARID1A protein levels by western blotting (C). Error bars denote standard deviation (SD). (D and E) The knockdown ability of each of the shRNAs was measured by examining the SMARCE1 mRNA levels by qRT-PCR (D) and the SMARCE1 protein levels by western blotting (E). Error bars denote standard deviation (SD).

FIG. 6 shows that restoration of SMARCE1 reverses the resistance to ALK inhibitors driven by SMACRE1 knockdown in EML4-ALK mutant NSCLC cells. (A) Ectopic expression of SMARCE1-ND that cannot be targeted by shSMARCE1 vectors re-sensitizes the SMARCE1 knockdown cells to ALK inhibitors. H3122 cells expressing pRS control or shSMARCE1 vectors were retrovirally infected with viruses containing pMX or pMX-SMARCE1-ND, and were grown in the absence or presence of 300 nM Crizotinib or 2.5 nM NVP-TAE684. Cells were then fixed, stained and photographed after 2 weeks (untreated) or 4 weeks (ALK inhibitors treatment). (B) The SMARCE1 protein levels in H3122 cells (untreated) described in FIG. 4A. (C and D) The endogenous SMARCE1 mRNA was measured by qRT-PCR using a 3′ UTR specific primer set (C) and the total SMARCE1 mRNA was measured by qRT-PCR using an ORF specific primer set.

FIG. 7 shows that restoration of Med12 reverses the resistance to EGFR inhibitor driven by MED12 knockdown in PC9 EGFR mutant cells. (A) Ectopic expression of mouse Med12 re-sensitizes the otherwise resistant MED12 knockdown cells to EGFR inhibitors. PC9 cells expressing pLKO control or shMED12 vectors were retrovirally infected with viruses containing pMX or pMX-Med12, and were grown in the absence or presence of 50 nM Gefitinib. Cells were then fixed, stained and photographed after 2 weeks (untreated) or 3 weeks (EGFR inhibitor treatment). (B) The MED12/Med12 protein levels in PC9 cells (untreated) described in FIG. 7A. (C and D) The endogenous MED12 mRNA (C) and the exogenous Med12 mRNA were measured by qRT-PCR.

FIG. 8 shows that suppression of MED12 confers drug resistance to EGFR inhibitors in H3255 EGFR mutant cells. (A) H3255 cells expressing shRNAs targeting MED12 are resistant to EGFR inhibitors. The functional phenotypes of shMED12 vectors are indicated by the colony formation assay in 25 nM Gefitnib or 25 nM Erlotinib. The cells were fixed, stained and photographed after 2 weeks (untreated) or 4 weeks (EGFR inhibitors treatment). (B) The knockdown ability of each of the shRNAs was measured by examining the MED12 mRNA levels by qRT-PCR. Error bars denote standard deviation (SD).

FIG. 9 shows that suppression of ARID1A confers drug resistance to EGFR and MET inhibitors in NSCLC cells with mutant EGFR or MET amplification. (A) PC9 cells expressing shRNAs targeting ARID1A are resistant to EGFR inhibitor. The functional phenotypes of shARID1A vectors are indicated by the colony formation assay in 25 nM Gefitinib. The cells were fixed, stained and photographed after 2 weeks (untreated) or 4 weeks (EGFR inhibitor treatment). (B) The ARID1A mRNA levels for the cells described in FIG. 9A were measured by qRT-PCR. Error bars denote standard deviation (SD). (C) H1993 cells expressing shRNAs targeting ARID1A are resistance to MET inhibitor. The functional phenotypes of shARID1A vectors are indicated by the colony formation assay in 200 nM Crizotinib. The cells were fixed, stained and photographed after 2 weeks (untreated) or 4 weeks (MET inhibitor treatment). (D) The ARID1A mRNA levels for the cells described in FIG. 9C were measured by qRT-PCR. Error bars denote standard deviation (SD).

FIG. 10 shows that restoration of SMARCE1 reverses the resistance to EGFR inhibitor driven by SMACRE1 knockdown in PC9 EGFR mutant cells. (A) Ectopic expression of SMARCE1-ND that cannot be targeted by shSMARCE1 vectors re-sensitizes the otherwise resistant SMARCE1 knockdown cells to EGFR inhibitor. PC9 cells expressing pRS control or shSMARCE1 vectors were retrovirally infected with viruses containing pMX or pMX-SMARCE1-ND, and were grown in the absence or presence of 50 nM Gefitinib. Cells were then fixed, stained and photographed after 2 weeks (untreated) or 4 weeks (EGFR inhibitor treatment). (B) The SMARCE1 protein levels in PC9 cells (untreated) described in FIG. 10A. (C and D) The endogenous SMARCE1 mRNA was measured by qRT-PCR using a 3′ UTR specific primer set (C) and the total SMARCE1 mRNA was measured by qRT-PCR using an ORF specific primer set.

FIG. 11 shows that restoration of SMARCE1 reverses the resistance to MET inhibitor driven by SMACRE1 knockdown in H1993.MET amplified cells. (A) Ectopic expression of SMARCE1-ND that cannot be targeted by shSMARCE1 vectors re-sensitizes the otherwise resistant SMARCE1 knockdown cells to MET inhibitor. H1993 cells expressing pRS control or shSMARCE1 vectors were retrovirally infected with viruses containing pMX or pMX-SMARCE1-ND, and were grown in the absence or presence of 200 nM Crizotinib. Cells were then fixed, stained and photographed after 2 weeks (untreated) or 4 weeks (MET inhibitor treatment). (B) The SMARCE1 protein levels in H1993 cells (untreated) described in FIG. 11A. (C and D) The endogenous SMARCE1 mRNA was measured by qRT-PCR using a 3′ UTR specific primer set (C) and the total SMARCE1 mRNA was measured by qRT-PCR using an ORF specific primer set.

FIG. 12 shows that restoration of SMARCE1 reverses the resistance to MET inhibitor driven by SMACRE1 knockdown in EBC1 MET amplified cells. (A) Ectopic expression of SMARCE1-ND that cannot be targeted by shSMARCE1 vectors re-sensitizes the otherwise resistant SMARCE1 knockdown cells to MET inhibitor. EBC1 cells expressing pRS control or shSMARCE1 vectors were retrovirally infected with viruses containing pMX or pMX-SMARCE1-ND, and were grown in the absence or presence of 200 nM Crizotinib. Cells were then fixed, stained and photographed after 2 weeks (untreated) or 4 weeks (MET inhibitor treatment). (B) The SMARCE1 protein levels in H1993 cells (untreated) described in FIG. 12A. (C and D) The endogenous SMARCE1 mRNA was measured by qRT-PCR using a 3′ UTR specific primer set (C) and the total SMARCE1 mRNA was measured by qRT-PCR using an ORF specific primer set.

FIG. 13 depicts a RAS-GAP RNAi screen that identifies DAB2IP and NF1 as critical determinants of drug sensitivity to EGFR inhibitors in EGFR mutant NSCLC cells. PC9 cells expressing controls (pLKO or shGFP) or 14 pools of shRNA vectors targeting each RAS-GAP were grown in the absence or presence of 50 nM Gefitinib or Elortinib. Cells were then fixed, stained and photographed after 2 weeks (untreated) or 4 weeks (EGFR inhibitors treatment).

FIG. 14 shows that suppression of DAB2IP confers drug resistance to EGFR inhibitors in PC9 EGFR mutant cells. (A) Validation of independent shRNAs (in pLKO vector) targeting DABP2IP in PC9 cells. The functional phenotypes of non-overlapping shDABP2IP vectors are indicated by the colony formation assay in 50 nM Gefitinib or Elortinib. The cells were fixed, stained and photographed after 2 weeks (untreated) or 4 weeks (EGFR inhibitors treatment). (B) The knockdown ability of each of the shRNAs was measured by examining the DAB2IP mRNA levels by qRT-PCR. Error bars denote standard deviation (SD). (C) Western blotting analysis of PC9 cells expressing controls (pLKO or shGFP) or shRNAs targeting DAB2IP treated with vehicle control or 25 nM Gefitinib for 8 hours.

FIG. 15 shows that suppression of NF1 confers drug resistance to EGFR inhibitors in PC9 EGFR mutant cells. (A) Validation of independent shRNAs (in pLKO vector) targeting NF1 in PC9 cells. The functional phenotypes of non-overlapping shNF1 vectors are indicated by the colony formation assay in 50 nM Gefitinib or Elortinib. The cells were fixed, stained and photographed after 2 weeks (untreated) or 4 weeks (EGFR inhibitors treatment). (B and C) The knockdown ability of each of the shRNAs was measured by examining the NF1 mRNA levels by qRT-PCR (B) and the NF1 protein levels by western blotting (C). Error bars denote standard deviation (SD).

FIG. 16 shows that suppression of MED12 and SMARCE1 leads to elevated phospho-ERK. (A) MED12^{KD} cells retain phospho-ERK levels in the presence of ALK inhibitor in EML4-ALK cells. H3122 cells expressing controls (pRS or shGFP) or shMED12 vectors were gown in the absence or presence of 20 nM NVP-TAE684 for 24 hours and the cell lysates were harvested for western blotting analysis. (B) SMARCE1^{KD} cells have elevated phospho-ERK in EML4-ALK cells. H3122 cells expressing controls (pRS or shGFP) or shSMARCE1 vectors were gown in the absence or presence of 20 nM NVP-TAE684 for 24 hours and the cell lysates were harvested for western blotting analysis. (C) MED12^{KD} cells have elevated phospho-ERK levels in EGFR mutant cells. PC9 cells expressing controls (pRS or shGFP) or shSMARCE1 vectors were gown in the absence or presence of 25 nM Gefitinib for 8 hours and the cell lysates were harvested for western blotting analysis.

FIG. 17 shows that MED12 suppression leads to ERK activation and confers multi-drug resistance in different cancer types (C and D) MED12 knockdown confers resistance to BRAF and MEK inhibitors in melanoma cells. C) BRAFV600E A375 cells expressing pLKO control or shMED12 vectors were cultured in the absence or presence of 2.5 μM PLX4032 or 0.5 μM AZD6244. The cells were fixed, stained and photographed after 10 (untreated) or 28 days (treated). D) MED12 suppression results in elevated level of p-ERK in melanoma cells. A375 cells expressing pLKO control or shMED12 vectors were grown in the absence or presence of 1 μM PLX4032 or 0.5 μM AZD6244 for 6 hours and the cell lysates were harvested for western blotting analysis. E-F) MED12 knockdown confers resistance to MEK inhibitor in colorectal cancer cells. E) KRASV12 SK-CO-1 cells expressing pLKO control or shMED12 vectors were cultured in the absence or presence of 0.5 μM AZD6244. The cells were fixed, stained and photographed after 14 (untreated) or 28 days (treated). F) MED12 suppression results in elevated level of p-ERK in colorectal cancer cells. SK-CO-1 cells expressing pLKO control or shMED12 vectors were grown in the absence or presence of 1 μM AZD6244 for 6 hours and the cell lysates were harvested for western blotting analysis. (G-H) Knockdown of MED12 confers resistance to multi-kinase inhibitor sorafenib in HCC Huh-7 cells. G) Colony formation assay of Huh-7 cells expressing pLKO control or shMED12 vectors (#4 and #5) were cultured in 2 μM sorafenib. The cells were fixed, stained and photographed after 14 (untreated) or 21 days (treated). H) MED12 suppression results in elevated level of p-ERK in HCC cells. Huh-7 cells expressing pLKO control or shMED12 vectors were grown in the absence or presence of 4 μM sorafenib for 6 hours and the cell lysates were harvested for western blotting analysis.

FIG. 18 shows that MED12 suppression confers multi-drug resistance in additional cell lines of different cancer types (A-B) Knockdown of MED12 confers resistance to EGFR inhibitor in NSCLC H3255 (EGFRL858R) cells. A) Colony formation assay of H3255 cells expressing pLKO control or shMED12 vectors (#4 and #5) were cultured in 25 nM gefininib. The cells were fixed, stained and photographed after 14 (untreated) or 28 days (treated). B) The level of knockdown of MED12 by each of the shRNAs was measured by examining the MED12 mRNA levels by qRT-PCR. Error bars denote SD. (C-D) knockdown of MED12 confers resistance to BRAF and MEK inhibitors in melanoma SK-MEL-28 (BRAFV600E) cells. C) Colony formation assay of SK-MEL-28 cells expressing pLKO control or shMED12 vectors (#4 and #5) were cultured in 5 μM PLX4032 or 1 μM AZD6244. The cells were fixed, stained and photographed after 14 (untreated) or 28 days (treated). D) The level of knockdown of MED12 by each of the shRNAs was measured by examining the MED12 mRNA levels by qRT-PCR. Error bars denote SD. (E-F) knockdown of MED12 confers resistance to BRAF and MEK inhibitors in CRC SW1417 (BRAFV600E) cells. E) Colony formation assay of SW1417 cells expressing pLKO control or shMED12 vectors (#4 and #5) were cultured in 2 μM PLX4032 or 150 nM AZD6244. The cells were fixed, stained and photographed after 14 (untreated) or 28 days (treated). F) The level of knockdown of MED12 by each of the shRNAs was measured by examining the MED12 mRNA levels by qRT-PCR. Error bars denote SD.

FIG. 19 shows that suppression of MED12 confers drug resistance to BRAF and MEK inhibitors in A375 melanoma cells. A375 (BRAFV600E) melanoma cells expressing shRNAs targeting MED12 are resistance to BRAF and MEK inhibitors. The functional phenotypes of shMED12 vectors are indicated by the colony formation assay in 5 uM PXL4720 or 12.5 nM PD-0325901. The cells were fixed, stained and photographed after 10 days (untreated) or 21 days (BARF and MEK inhibitors treatment).

FIG. 20 shows that suppression of TGFβR2 restores the sensitivity to ALK inhibitors in MED12^{KD} cells.

FIG. 21 shows that TGFβ signaling is required for the drug resistance driven by MED12 suppression A) Schematic outline of the “drop out” RNAi screen for kinases whose inhibition restores sensitivity to crizotinib in MED12KD cells. Human TRC kinome shRNA library polyclonal virus was produced to infect H3122 cells stably expressing shMED12#3, which were then left untreated (control) or treated with 300 nM crizotinib for 10 days. After selection, shRNA inserts from both populations were recovered by PCR and identified by next generation sequencing. B) Representation of the relative abundance of the shRNA bar code sequences from the shRNA screen experiment depicted in panel A. The y-axis is enrichment (relative abundance of crizotinib treated/untreated) and x-axis is the intensity (average sequence:reads in untreated sample) of each shRNA. Among the 51 top shRNA candidates (more than 2.5-fold depleted by crizotinib treatment and more than 200 reads in untreated as indicated by the red dash lines), two independent shTGFβR2 vectors (in light-gray near end of arrow points) were identified. C) Suppression of TGFβR2 restores the crizotinib sensitivity in MED12KD cells. Using lentiviral infection, pLKO control or two independent shTGFβR2 vectors were introduced into H3122 control or MED12KD cells. After this, cells were cultured in the absence or presence of 300 nM crizotinib. The cells were fixed, stained and photographed after 14 (untreated) or 21 days (treated). D) The level of knockdown of TGFβR2 by each of the shRNAs was measured by examining the MED12 mRNA levels by qRT-PCR. Error bars denote SD.

FIG. 22 shows that TGFβ treatment confers resistance to ALK inhibitors in EML4-ALK NSCLC cells. Activation of TGFβ signaling is sufficient to confer resistance to ALK inhibitors in EML4-ALK cells.

FIG. 23 shows that TGFβ treatment confers resistance to EGFR inhibitors in EGFR mutant NSCLC cells. Activation of TGFβ signaling is sufficient to confer resistance to EGFR inhibitors.

FIG. 24 shows that TGFβ activation is sufficient to confer multi targeted drug resistance in different cancer types. Recombinant TGFβ treatment leads to resistance to crizotinib in H3122 cells (A), AZD6244 in SK-CO-1 cells (C) and PLX4032 and AZD6244 in A375 cells (D) in a TGFβ-dosage dependent manner.

FIG. 25 shows that MED12^{KD} and TGFβ treatment both lead to elevated phosphor-ERK.

FIG. 26 shows that morphological changes in MED12^{KD} cells resemble those of TGFβ.

FIG. 27 shows that MED12KD cells morphologically resemble the cells treated with recombinant TGFβ Photographs of Huh-7 (B) cells expressing pLKO control or shMED12 and the control cells treated with recombinant 50 μM of TGFβ. Bar, 25 μm.

FIG. 28 is a microarray analysis showing up-regulation of TGFβ target genes in MED12^{KD} cells.

FIG. 29 shows that MED12 suppresses TGFβ signaling by negatively regulating TGFβR2 (A-F) Downregulation of MED12 leads to induction of a panel of TGFβ target genes and EMT marker genes. mRNA expression analysis by qRT-PCR of TGFβ target genes ANGPTL4 (A), TAGLN (B), CYR61 (C) and CTGF (D) and EMT marker genes VIM (E) and CDH2 (F) in H3122 and PC9 cells expressing pLKO controls or shRNAs targeting MED12. Cells were cultured in normal condition without TGFβ stimulation. Error bars denote SD. (G-H) MED12 suppression results in strong induction of TGFβR2 protein and SMAD2 phosphorylation. Western blot analysis of H3122 (G) and PC9 (H) cells expressing pLKO control or shMED12 vectors. HSP90 was used as a loading control. I) MED12 localizes to both nucleus and cytoplasm. Western blotting analysis of the nuclear and cytoplasmic fractions prepared from PC9 cells expressing control vector or shMED12 with or without 16 hours of 25 nM gefitinib treatment. Lamin A/C and SP1 were used as marker controls for nuclear fractions, while α-TUBULIN and HSP90 were used as controls for cytoplasmic fractions. J) MED12 is capable of physically interacting with TGFβR2. Western blotting analysis of coimmunoprecipitation experiments using Phoenix cells cotransfected with TGFβR2 and MED12 in a ratio of 5:1.

FIG. 30 shows that MED12 suppresses TGF β signaling by negatively regulating TGF β receptor signaling in additional cell line models (A-F) Downregulation of MED12 leads to induction of a panel of TGFβ target genes and EMT marker genes. mRNA expression analysis by qRT-PCR of TGF β target genes ANGPTL4 (A), TAGLN (B), CYR61 (C) and CTGF (D) and EMT marker genes VIM (E) and CDH2 (F) in A375, SK-CO-1 and Huh-7 cells expressing pLKO controls or shMED12. Cells were cultured in normal condition without TGFβ stimulation. Error bars denote SD. (G) mRNA levels of TGFβR2 in H3122, PC9, A375, SK-CO-1 and Huh-7 cells expressing pLKO control or shMED12 were documented by qRT-PCR. Error bars denote SD. (H-I) MED12 suppression results in strong induction of TGFβR2 protein and SMAD2 phosphorylation. Western blot analysis of A375 (H) and SK-CO-1 (I) cells expressing pLKO control or shMED12 vectors. α-TUBULIN was used as a loading control. J) MED12 localizes to both nucleus and cytoplasm. Western blotting analysis of the nuclear and cytoplasmic fractions prepared from H3122 cells expressing control vector or shMED12 with or without 16 hours of 300 nM crizotinib treatment. Lamin A/C and SP1 were used as marker controls for nuclear fractions, while α-TUBULIN and HSP90 were used as controls for cycloplasmic fractions. K) Western blotting showing that MED12 knockdown leads to induction of mesenchymal markers Vimentin and N-cadherin in Huh-7 cells.

FIG. 31 shows that activation of RAS/ERK pathway confers resistance to tyrosine kinase inhibitors in NSCLC cells.

FIG. 32 is a table showing that SWI/SNF and MEDIATOR complexes regulate resistance to a variety of targeted cancer drugs.

FIG. 33 shows that MED12KD signature overlaps with an EMT signature and predicts poor outcome in CRC and drug response to MEK inhibitors A) Genes that are frequently upregulated upon MED12 knockdown from the MED12KD signature significantly overlap with a list of genes upregulated during EMT (p=8.9*10-23; see Experimental Procedures). p=hypergeometric p-value. B) Kaplan-Meier analysis of disease specific survival (DSS) for the cohort of 231 CRC. MED12KD gene signature was used to hierarchically cluster the 231 CRC tumors into a cluster with poor DSS (cluster 1, black (bottom) line) and one with significantly better DSS (cluster 2, gray (top) line). C) MED12KD signature predicts drug responses to MEK inhibitors in 152 cell lines of different cancer types harboring the matching RAS or RAF mutations. High expression of subsets of genes upregulated in the MED12KD signature is significantly associated with higher IC50s for all four MEK inhibitors in (AZD6244, p-=0009; CI-1040, p=0.004; PD-0325901, p=0.007; RDEA119, p=0.013). Across these gene sets, each cell line was scored for the percentage of times it had high expression of the gene as well as being resistant to the inhibitor. The heatmap in the left panel of this figure depicts this percentage for each MEK inhibitor. The cell lines are sorted using hierarchical clustering for visualization. The middle and right panel depict the tissue type of the cell lines and their RAS/RAF mutation status.

FIG. 34 shows that IC50 values for AZD6244 and expression levels for ZBED2 across the 152 RAF/RAS mutated lines.

The top panel represents a histogram of IC50 values for the MEK inhibitor, AZD6244, across the 152 cell lines. Below the histogram, the individual IC50 values are plotted using squares (sensitive cell lines) and circles (resistant cell lines). The panel on the left depicts the histogram for the expression levels of gene ZBED2. To the right of the histogram, the individual expression levels are plotted using plus signs (upregulated), crosses (normal expression) and stars (downregulated). The scatter plot depicts the IC50 values and gene expression for each cell line. In this case, there are significantly many cell lines that show resistance to AZD6244 and are upregulated for ZBED2. These cell lines are found in the top-right area of the scatter plot and are indicated by plus signs inside of circles. The MED12 knockdown signature contains a significantly large number of such genes indicating the potential predictive value of this signature.

FIG. 35 shows that TGFβR inhibitor and TKIs synergize to suppress proliferation of MED12^{KD} NSCLC cells. A) Combination of TGFβR and ALK inhibitors synergistically inhibits growth of MED12KD NSCLC cells harboring EML4-ALK translocation. H3122 cells expressing pRS control or shMED12 vectors were cultured in the absence and the presence of 1 μM LY2157299, 300 nM crizotinib, or the combination of 1 μM LY2157299 and 300 nM crizotinib. The cells were fixed, stained and photographed after 14 (untreated and LY2157299 alone) or 28 days (crizotinib alone and LY2157299 plus crizotinib). B) Combination of TGFβR and EGFR inhibitors synergistically inhibits growth of MED12KD NSCLC cells harboring EGFR activating mutation. PC9 cells expressing pLKO control or shMED12 vectors were cultured in the absence and the presence of 1 μM LY2157299, 100 nM gefitinib, or the combination of 1 μM LY2157299 and 100 nM gefitinib. The cells were fixed, stained and photographed after 10 (untreated and LY2157299 alone) or 28 days (gefitinib alone and LY2157299 plus gefitinib).

FIG. 36 is a table depicting kinases screened for kinases whose inhibition restores sensitivity to crizotinib in MED12KD cells. Listed are the gene symbols for the genes tested in the “drop out” RNAi screen and the number of shRNAs for each gene present in the library.

FIG. 37 is a table depicting MED12KD signature gene list. Listed are genes deregulated by MED12KD (>2 fold) in at least three out five cell lines (H3122, PC9, SK-CO-1, A375 and Huh-7).

FIG. 38 is a table depicting. EMT signature gene list. Listed are genes of an EMT signature that was created by combining published EMT expression signatures as described herein.

FIG. 39 is a table depicting overlapping genes between MED12KD and EMT signatures. Listed are overlapping genes that are upregulated in both the MED12KD and EMT signatures.

FIG. 40 is a table depicting MED12KD signature genes that are significantly associated with higher IC50s for MEK inhibitors in the 152 cell lines. Of the 237 genes that were upregulated by MED12KD as identified by RNA-Seq, Applicants could read the expression levels for 170 genes in these 152 cell lines that have activating mutations in RAS or BRAF. High expression of subsets of these 170 genes is significantly associated with higher IC50s for all four MEK inhibitors in these cell lines.

FIG. 41 is a table depicting 152 tumor cell lines used for the COSMIC Cell Line Panel Analysis. Listed are 152 COSMIC cell lines that have activating mutations in RAS or BRAF and their drug response data (IC50 values) to four MEK inhibitors.

### DETAILED DESCRIPTION

The instant invention provides methods and related compositions pertaining to the identification of a tumor that will be resistant to treatment by a certain compound or class of compounds. In certain embodiments, the invention provides one or more markers for resistance to anticancer treatment in a patient. In some embodiments, the marker is a MEDIATOR complex and/or SWI/SNF complex gene.

Examples of MEDIATOR complex genes that may serve as a marker for resistance to anticancer treatment in a patient as described herein include MED22, MED11, MED17, MED20, MED30, MED19, MED18, MED8, MED6, MED28, MED9, MED21, MED4, MED7, MED31, MED10, MED1, MED26, MED2, MED3, MED25, MED23, MED5, MED14, MED16, MED15, CycC, CDK8, MED13, MED12, MED13L, and MED12L (see e.g., MED12L Gene ID: 116931 available from the National Center for Biotechnology Information (NCBI) website). See, e.g., Malik, S, Roeder, R G, “The metazoan Mediator co-activator complex as an integrative hub for transcriptional regulation” Nat Rev Genet. (2010) 11(11):761-72.

Examples of SWI/SNF complex genes that may serve as a marker for resistance to anticancer treatment in a patient as described herein include ARID1A, ARID1B, ARID2, SMARCA2, SMARCA4, PBRM1, SMARCC2, SMARCC1, SMARCD1, SMARCD2, SMARCD3, SMARCE1, ACTL6A, ACTL6B, and SMARCB1. See, e.g., Reisman, D et al. “The SWI/SNF complex and cancer” Oncogene. (2009) 28(14):1653-68.

In some embodiments, the invention provides methods whereby measurement of reduced expression of a MEDIATOR complex and/or SWI/SNF complex gene in one or more cancer cells of a patient identifies these cancer cells as cells that may be resistant to treatment by one or more receptor tyrosine kinase (RTK) inhibitors. RTKs are involved in a number of diverse physiological processes, including proliferation and differentiation, cell survival and metabolism, cell migration, and cell-cycle control (see, e.g., Lemmon, M A, Schlessinger, J “Cell Signaling by Receptor Tyrosine Kinases” Cell (2010) 141:1117-1134).

In addition, an overview of non-small cell lung cancer signaling pathways may be found at www(dot)n-of-one(dot)com/cancer-news-info/egfr/ and the figure presented therein adapted from Herbst, et al. NEJM 2008.

Described herein is the use of a large-scale loss-of-function genetic screen to identify genes whose suppression can confer resistance to crizotinib in a NSCLC cell line harboring an EML4-ALK translocation. Applicants identify a key component of the transcriptional MEDIATOR complex, MED12, as a determinant of crizotinib response in NSCLC. Remarkably, Applicants find that suppression of MED12 also confers resistance to a range of targeted cancer drugs in other cancer types as well, including colon cancer, melanoma and liver cancer. Applicants identify an unexpected activity of MED12 in regulating TGFβ receptor signaling, as the major mechanism of drug resistance induction.

Applicants identify herein MED12 as a candidate biomarker of response to a range of targeted cancer drugs in a variety of cancer types through a previously unappreciated role of this protein in TGFβ receptor signaling. MED12 is a component of the MEDIATOR transcriptional adapter complex that serves as a molecular bridge between the basal transcription machinery and its upstream activators (Conaway et al., 2005). More specifically, MED12 is a subunit of the “kinase” module of the MEDIATOR complex, which also contains MED13, CYCLIN C and CDK8, whose gene sequence is amplified in some 50% of colon cancers (Firestein et al., 2008). The involvement of MEDIATOR components in responses to TKIs was unexpected, as most of the known genes that influence responses to TKIs involve components of signaling pathways that act downstream or in parallel of these receptors. Applicants reconcile this apparent discrepancy by demonstrating that part of MED12 also resides in the cytosol, where it interacts with the TGFβ type II receptor to inhibit its activity. Consequently, downregulation of MED12 by RNAi strongly activates TGFβ signaling, as evidenced by phosphorylation of SMAD2 and induction of many canonical TGFβ target genes. Activation of TGFβ signaling has been linked previously to activation of ERK signaling (reviewed by (Zhang, 2009)). Consistent with this, Applicants observed activation of ERK signaling by MED12 suppression, which persists in the presence of drugs like crizotinib, gefitinib, vemurafenib, seluteminib and sorafenib (FIGS. 17, 18 and data not shown), thus providing a rationale for why suppression of MED12 confers resistance to these drugs.

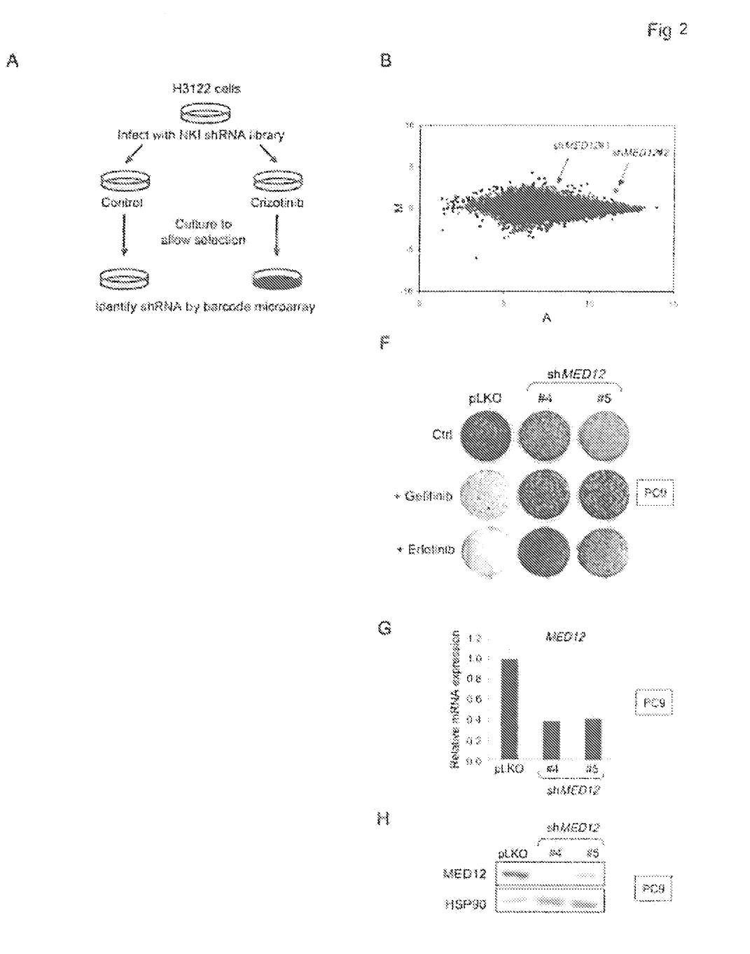

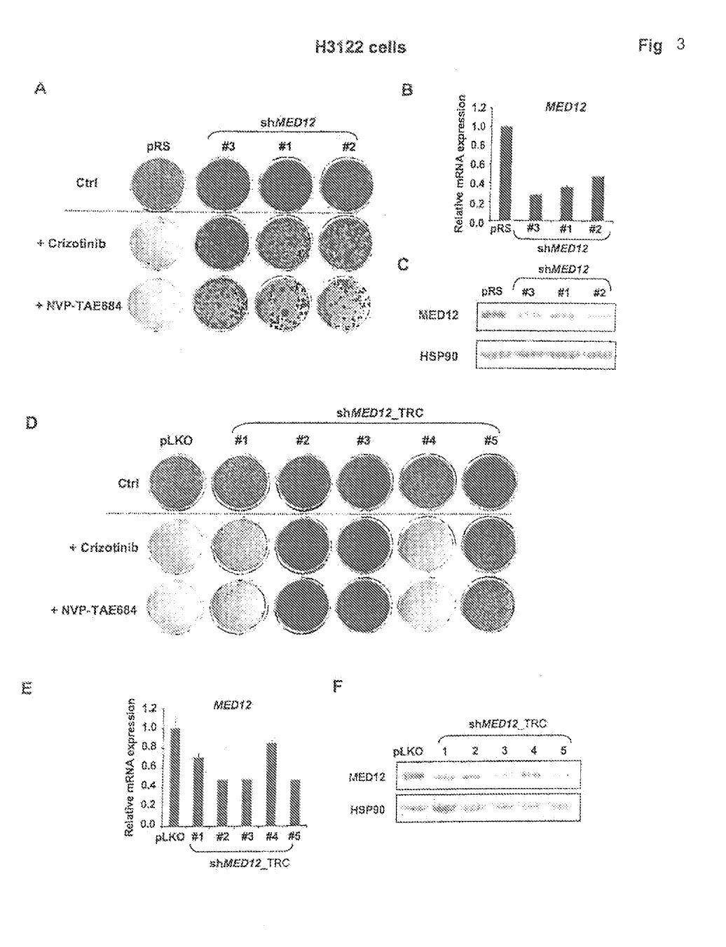

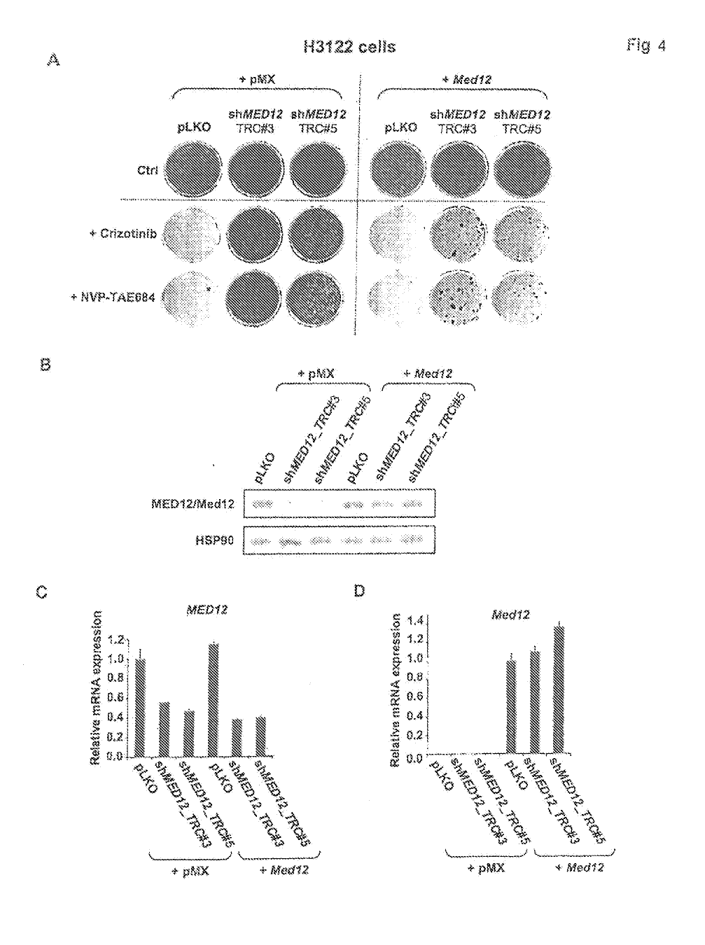

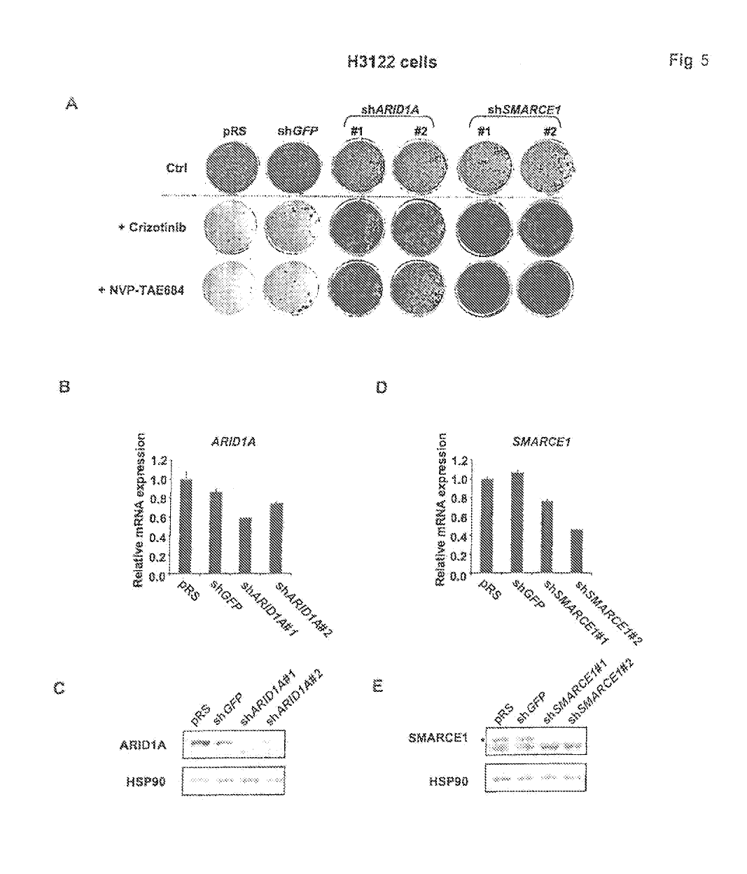

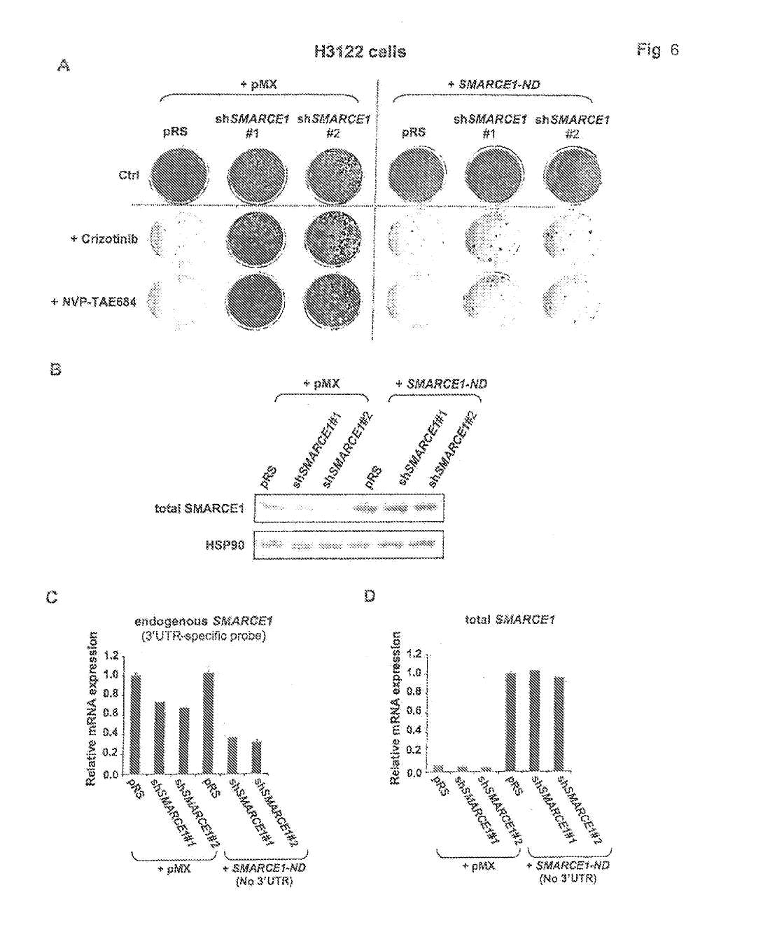

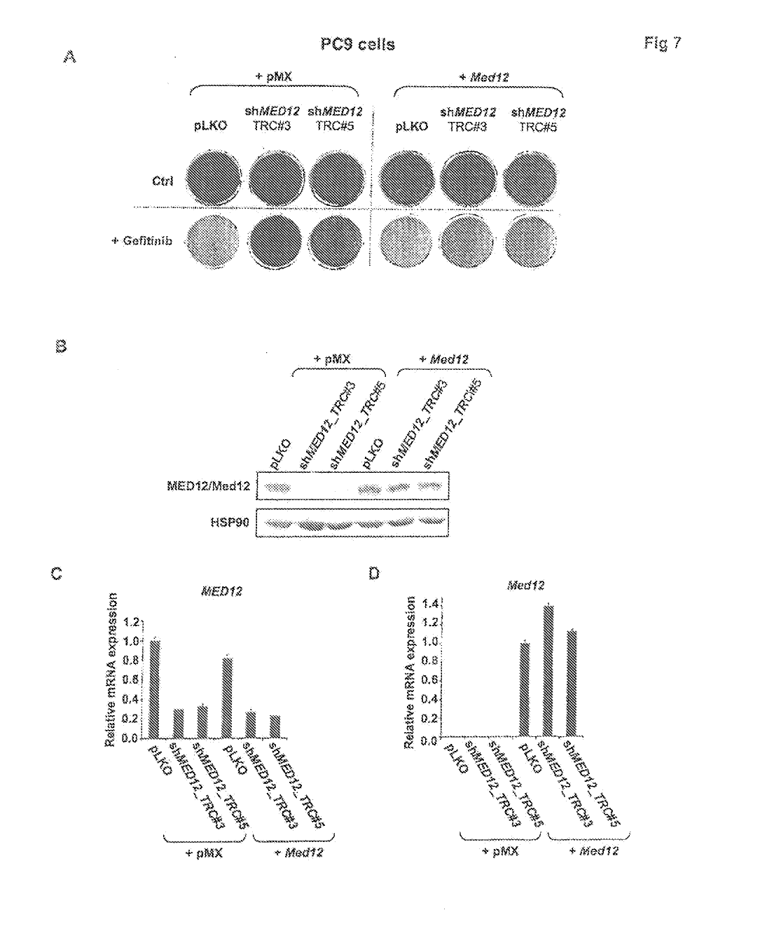

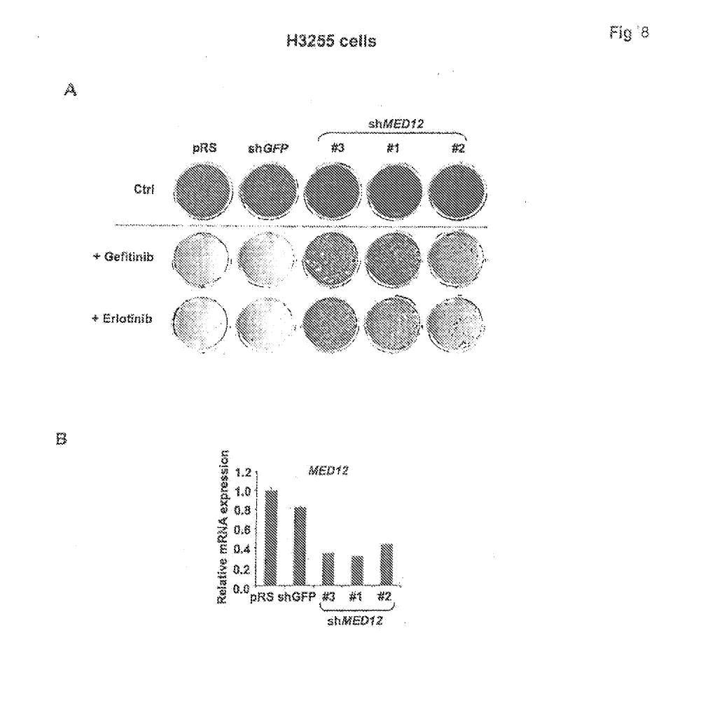

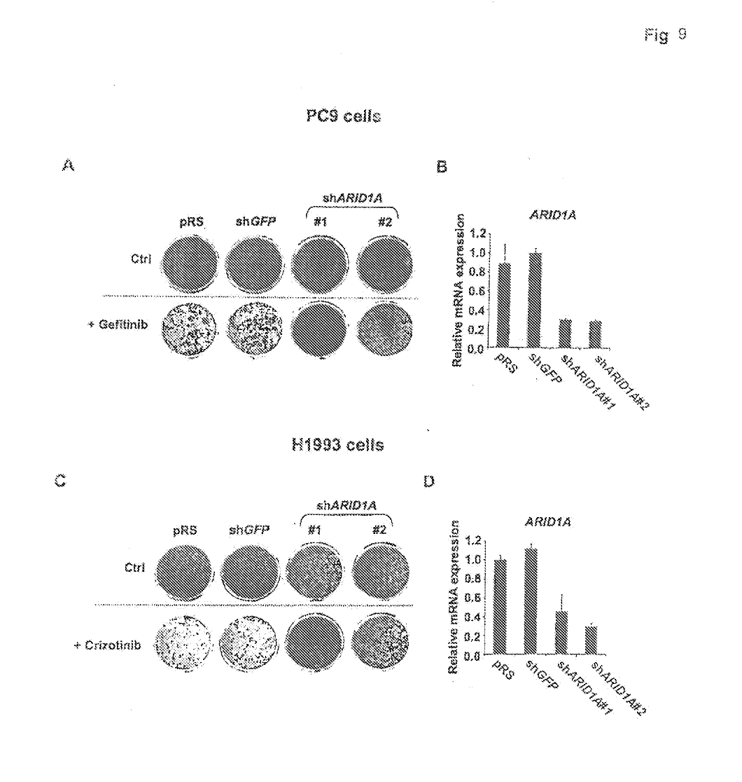

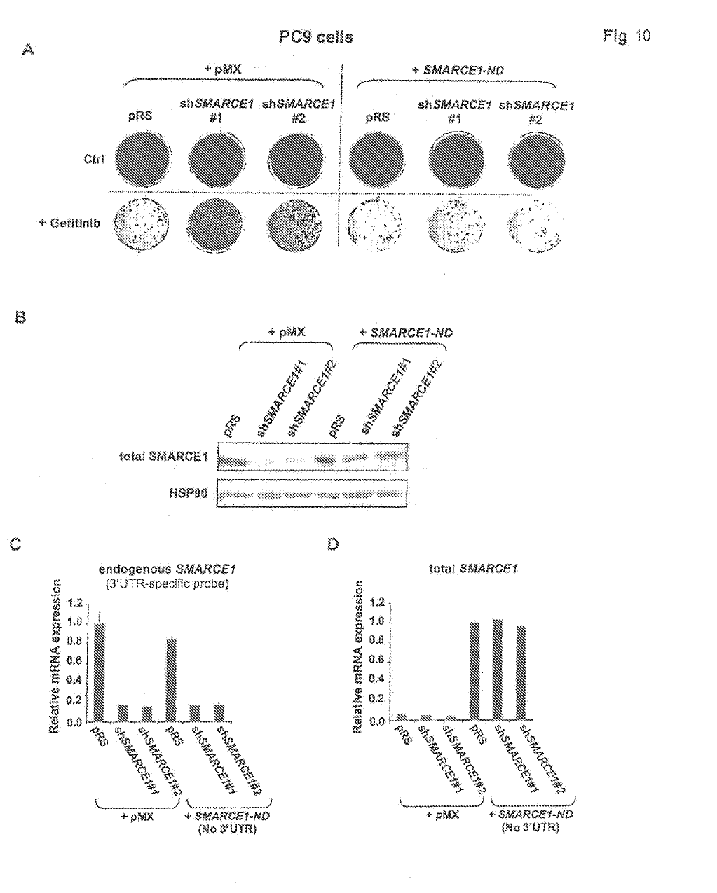

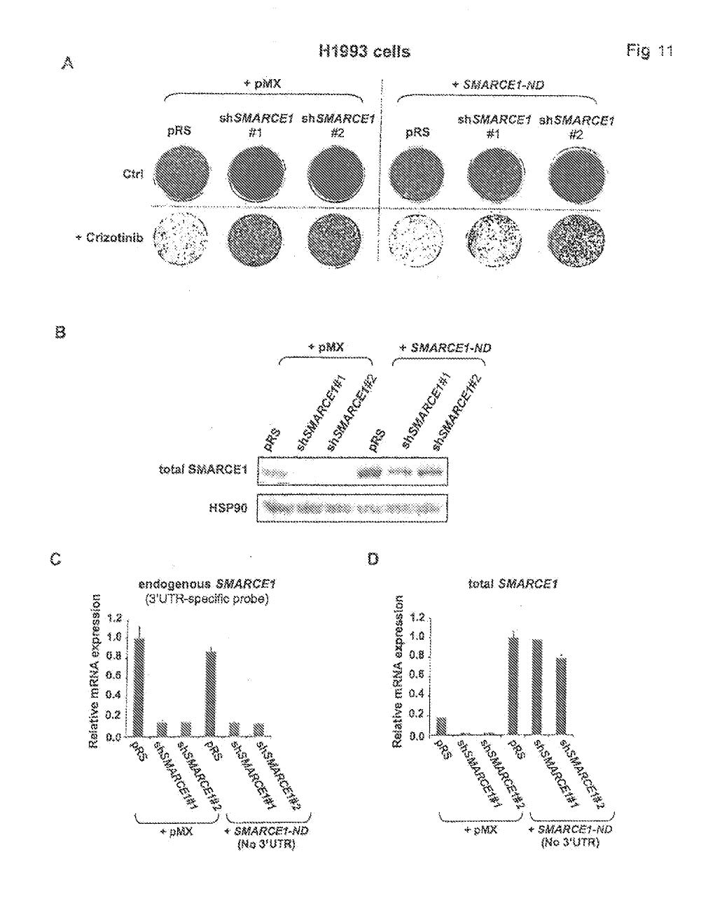

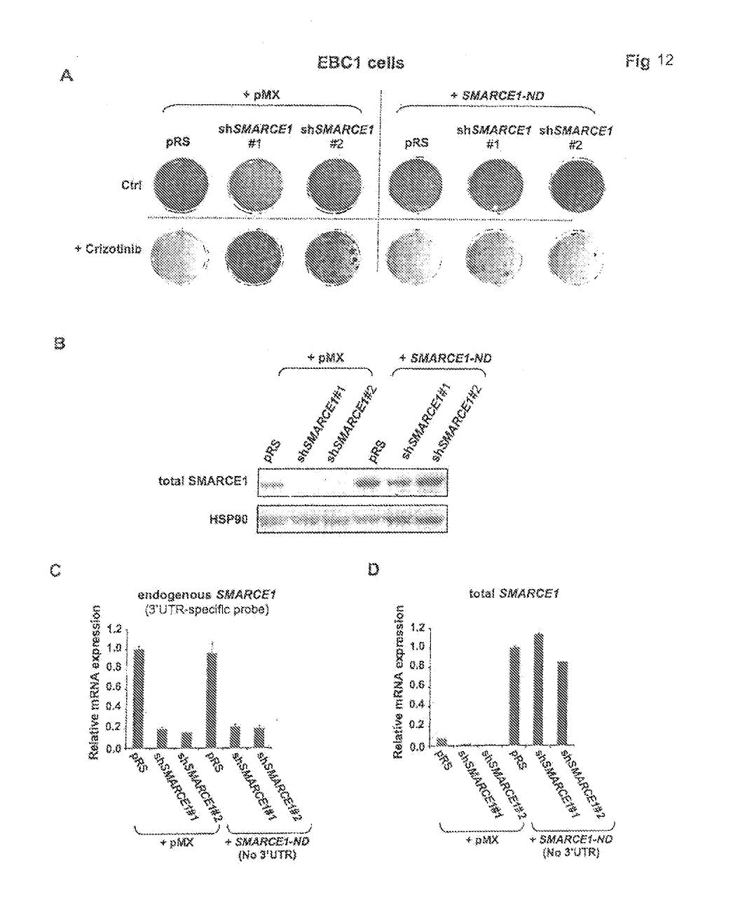

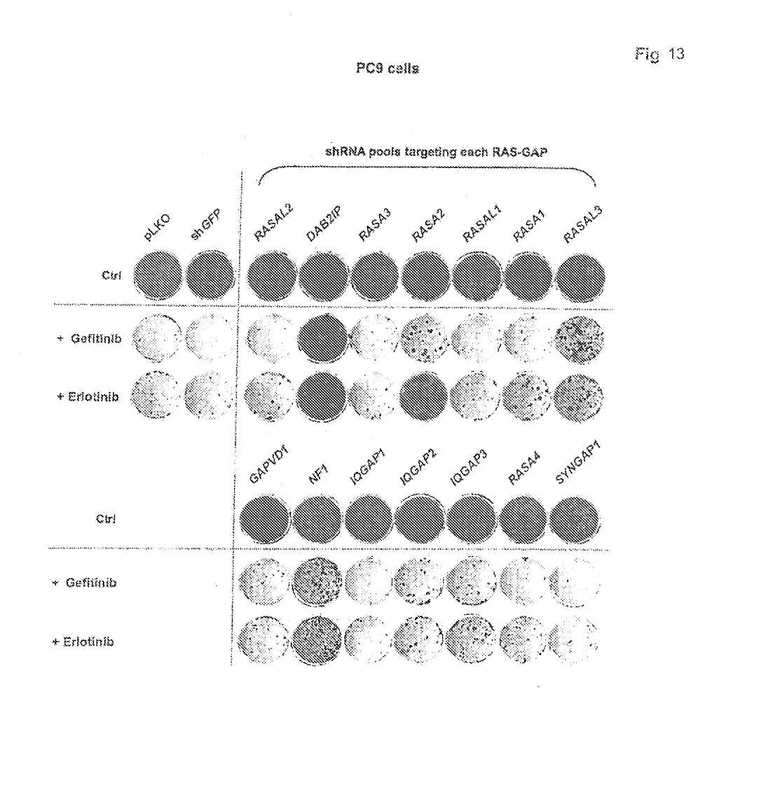

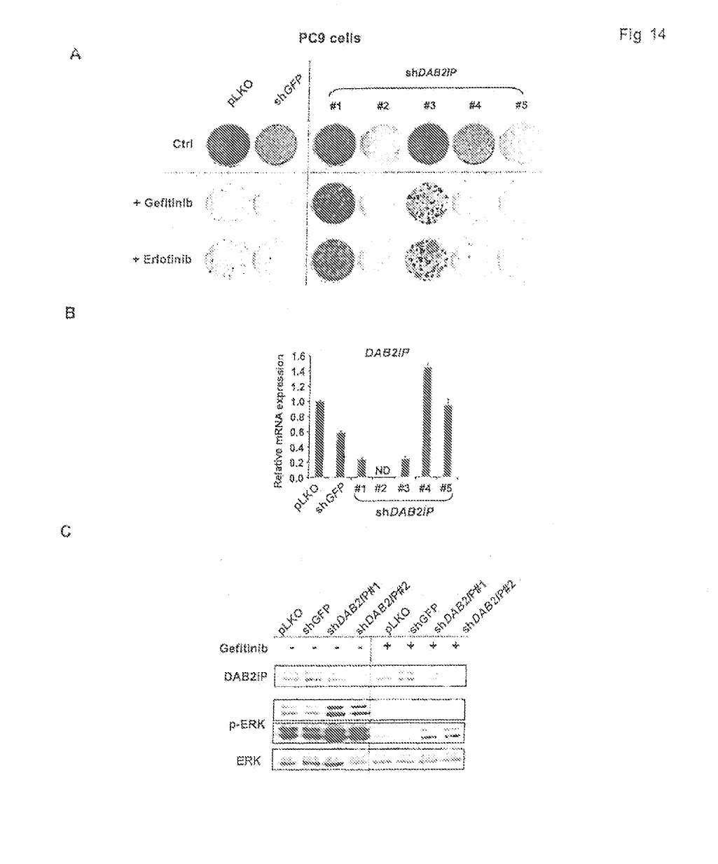

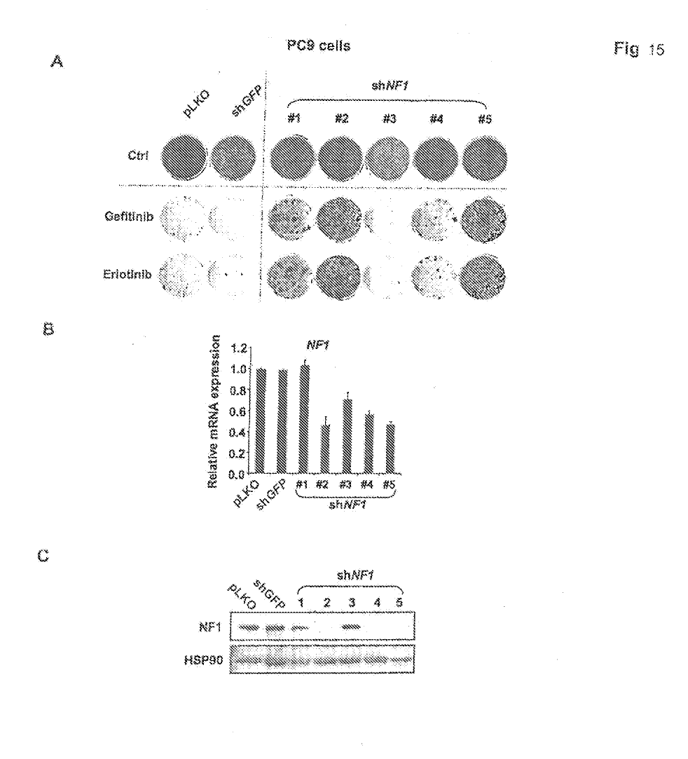

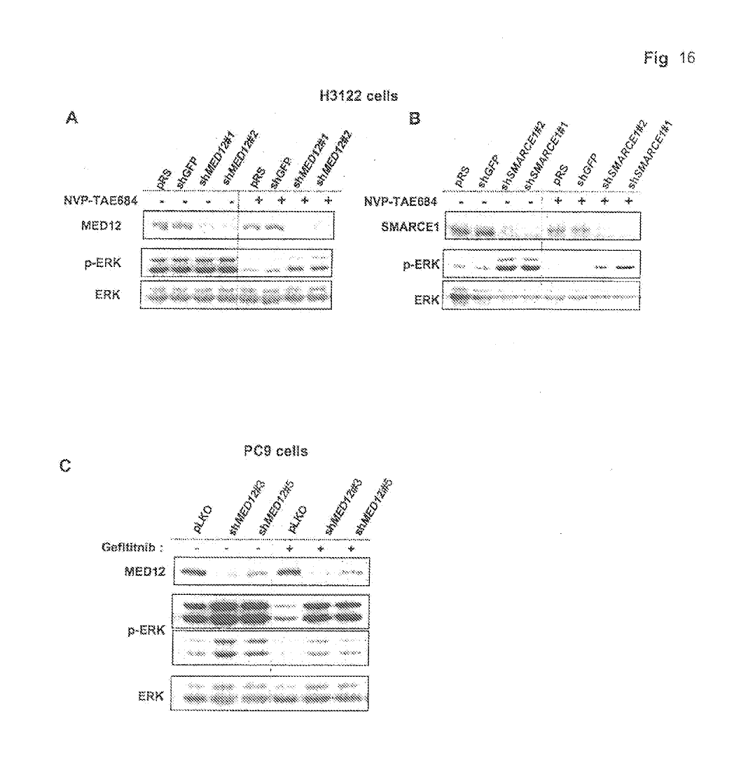

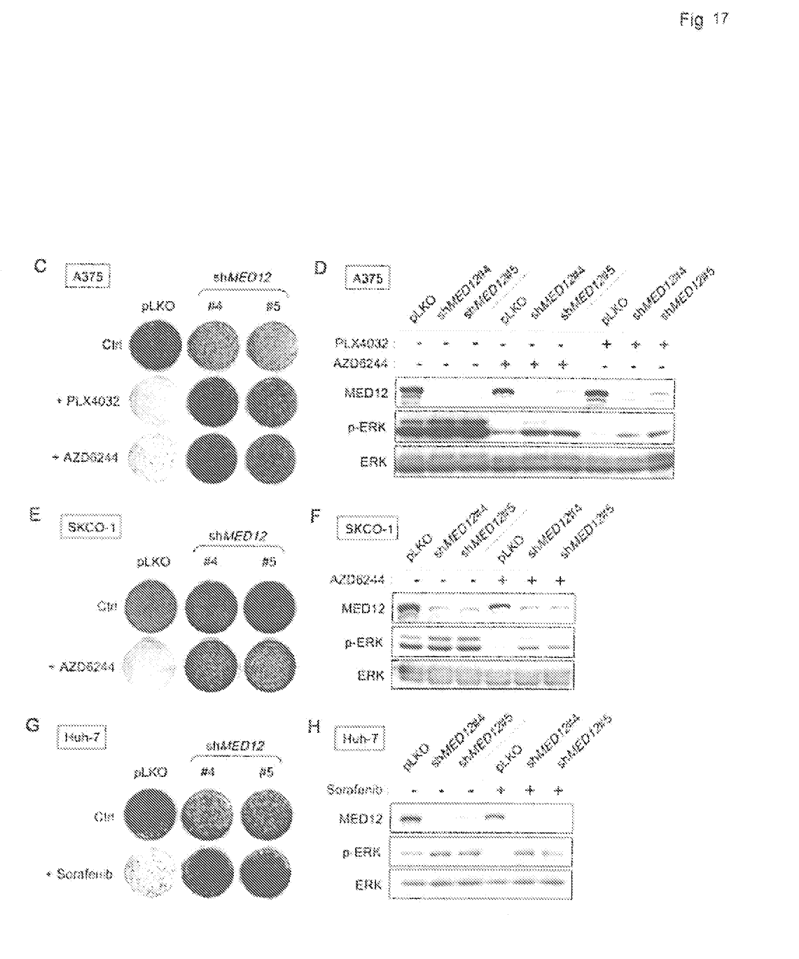

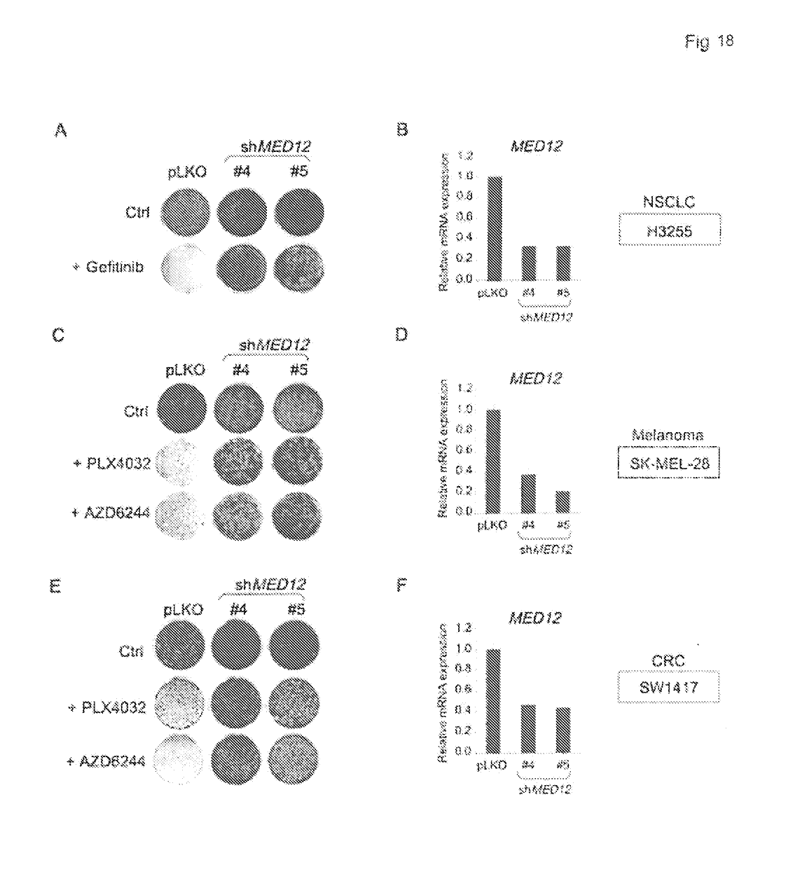

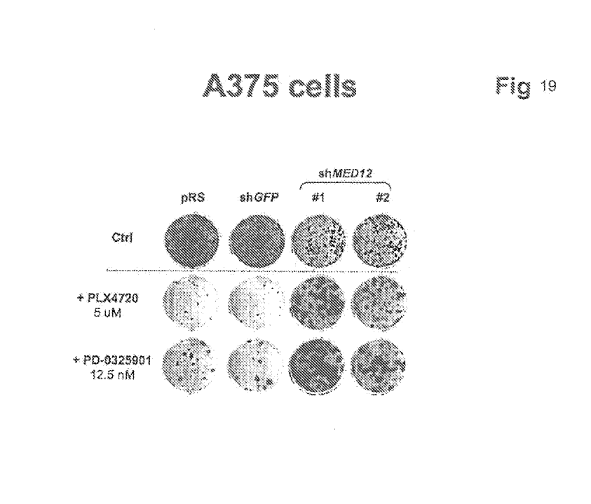

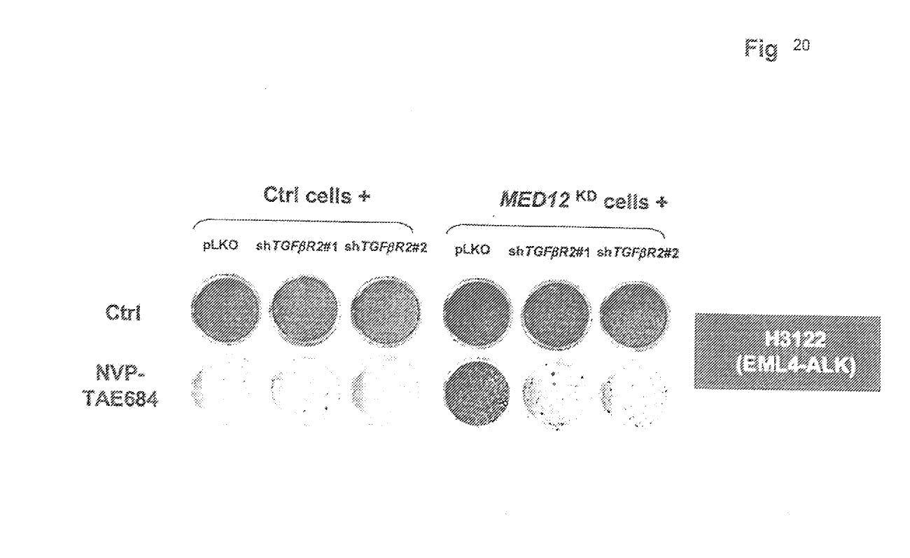

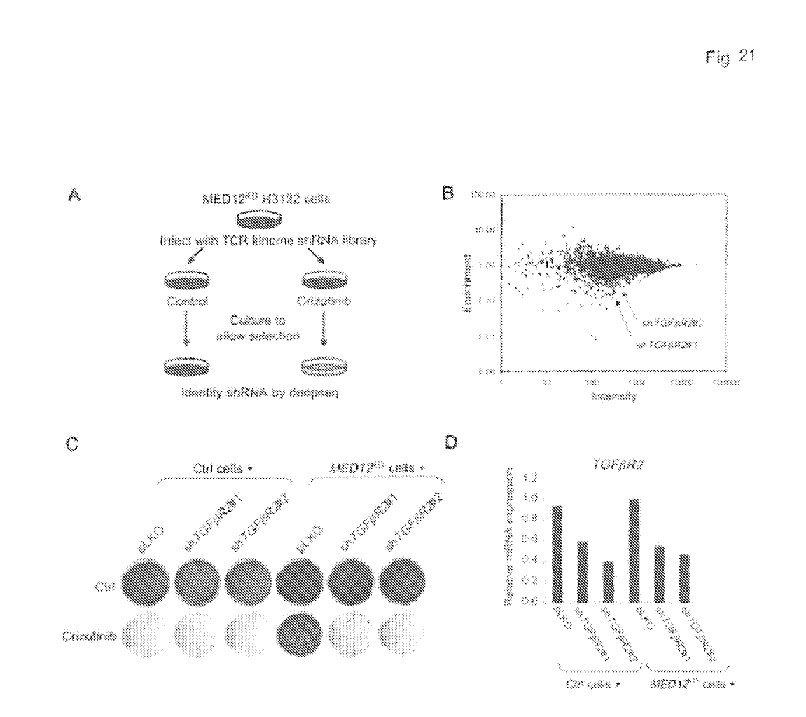

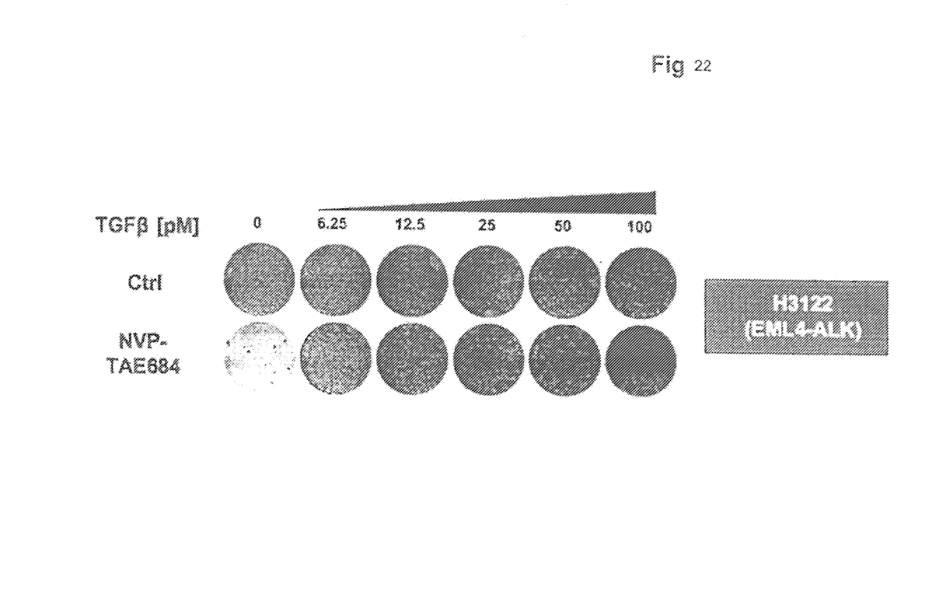

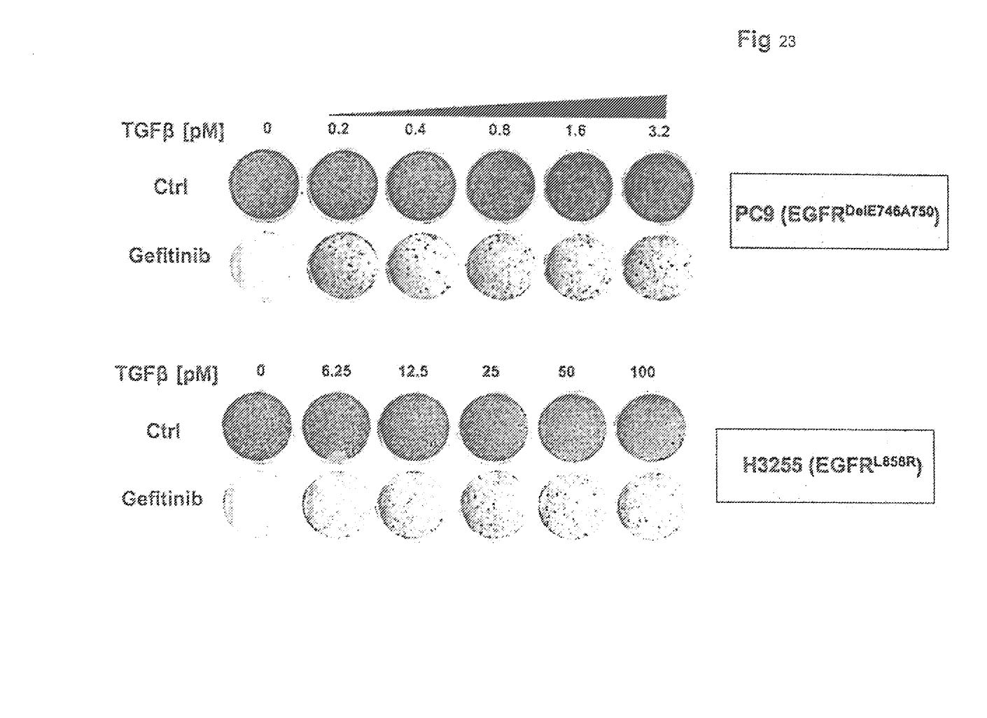

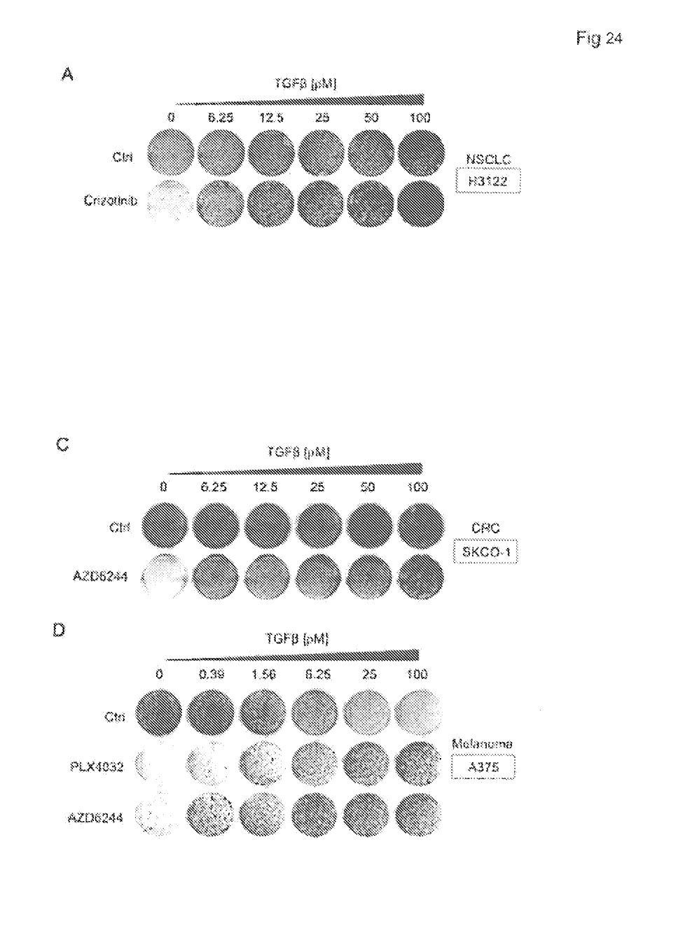

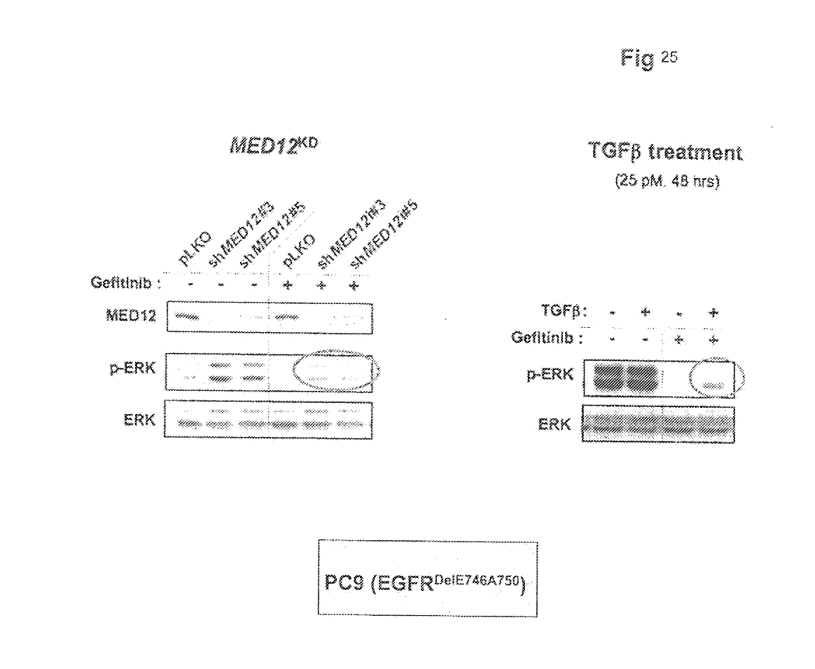

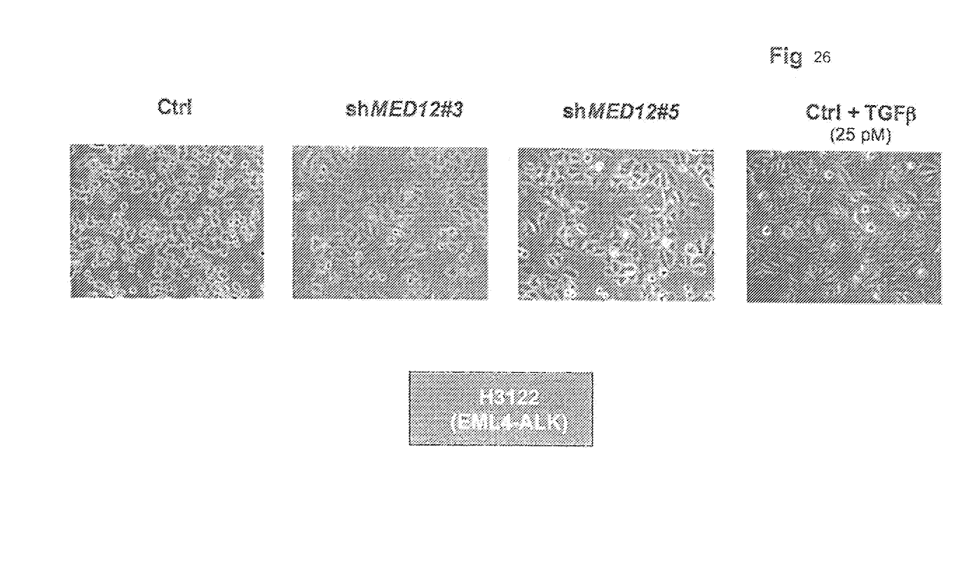

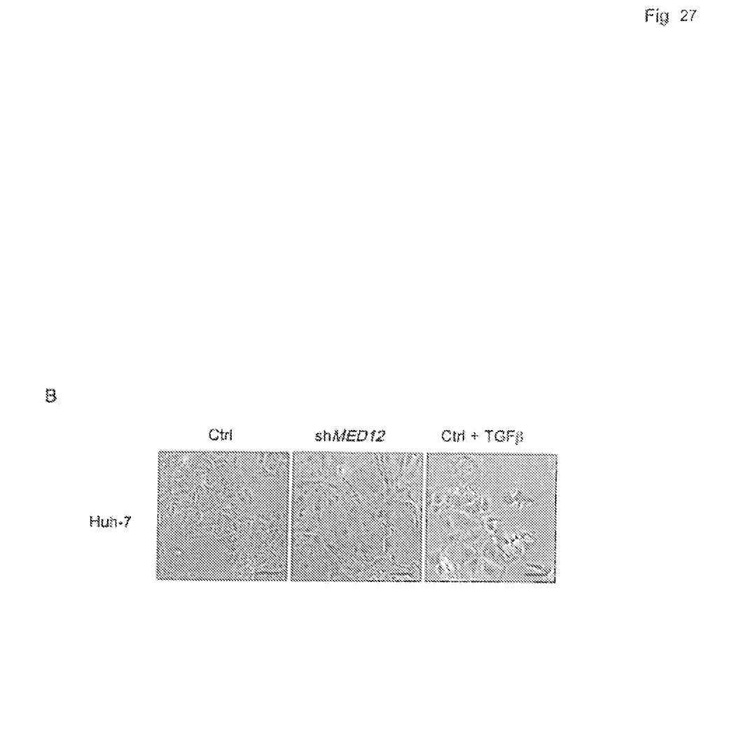

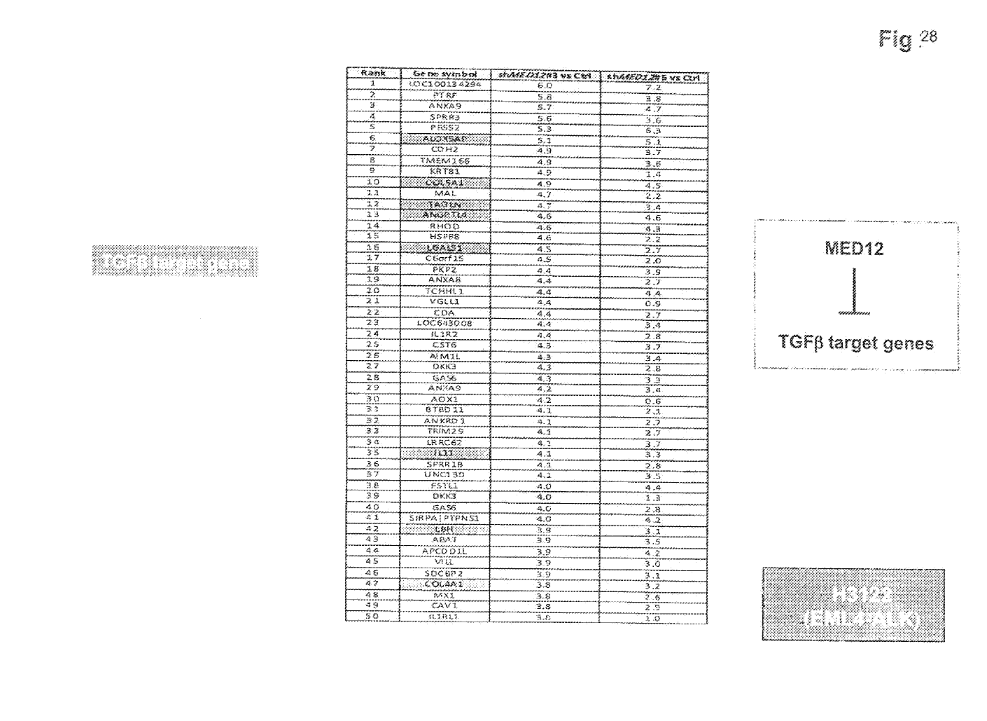

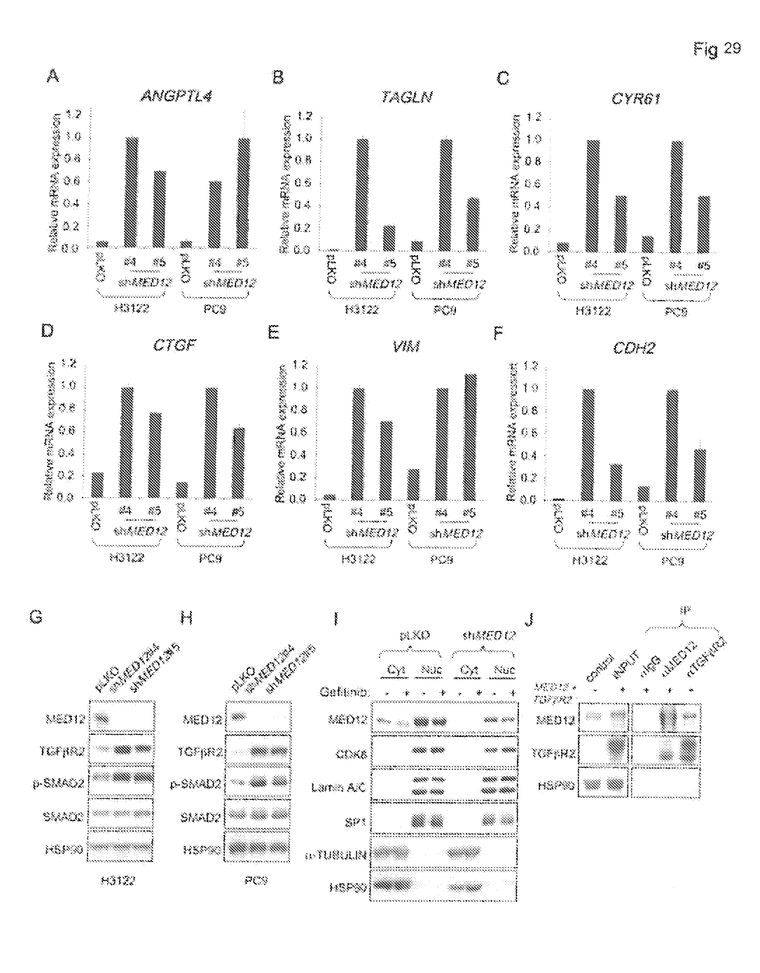

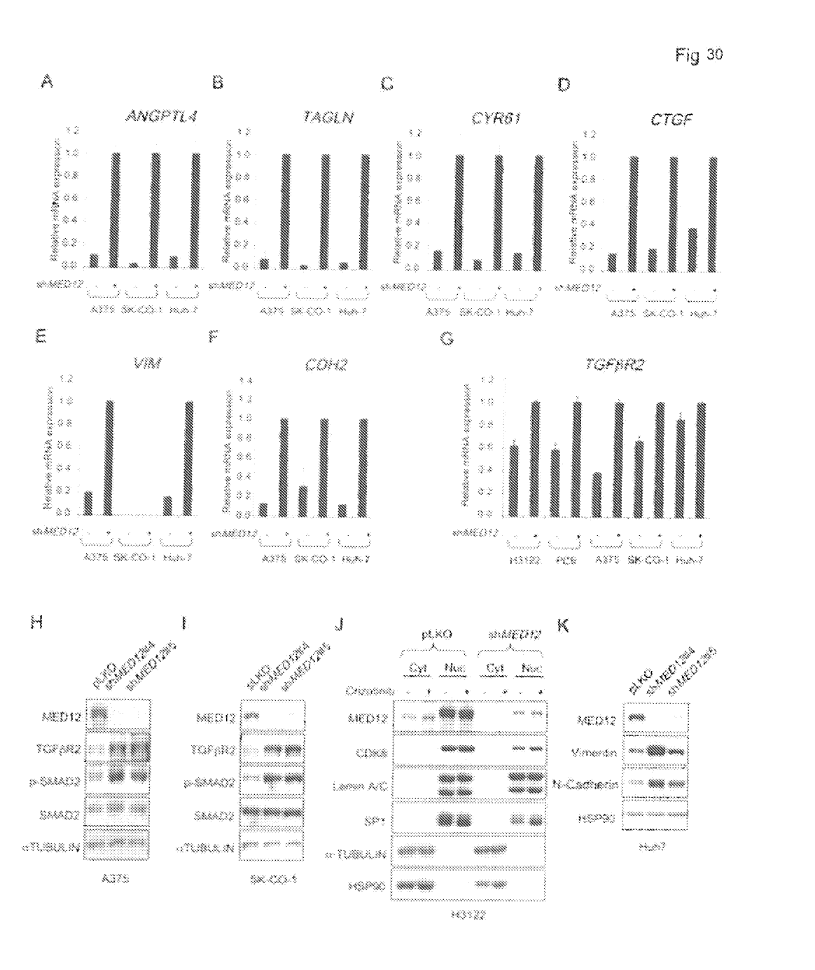

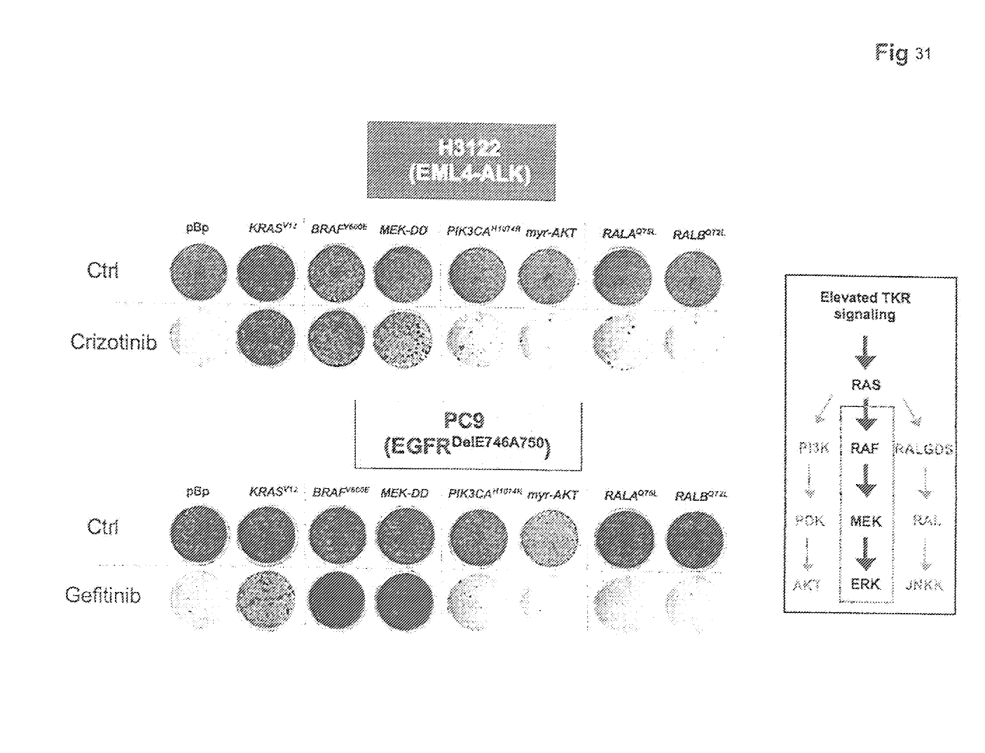

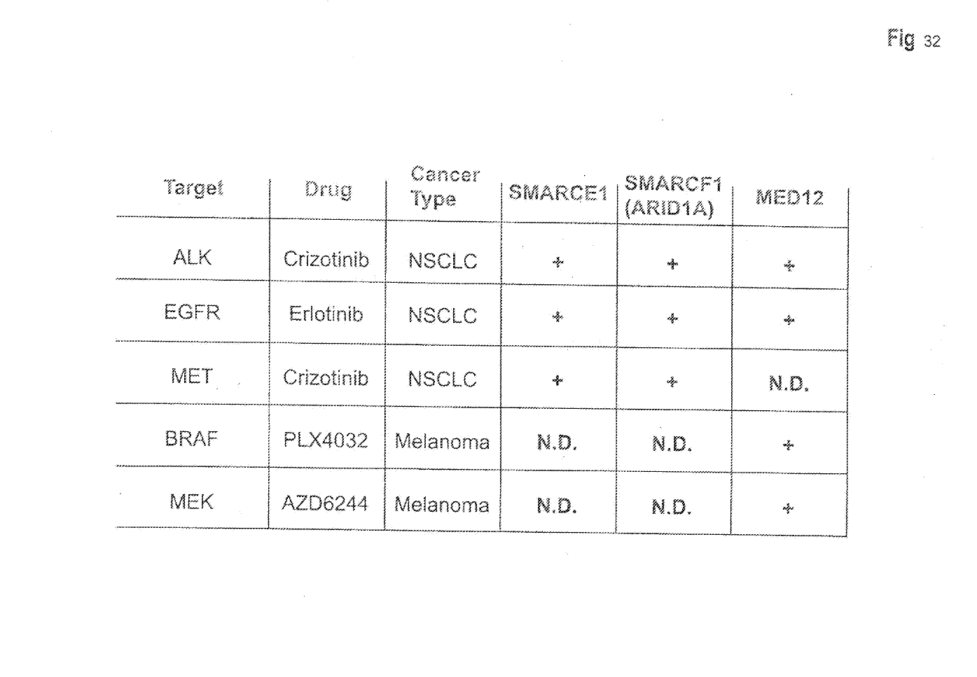

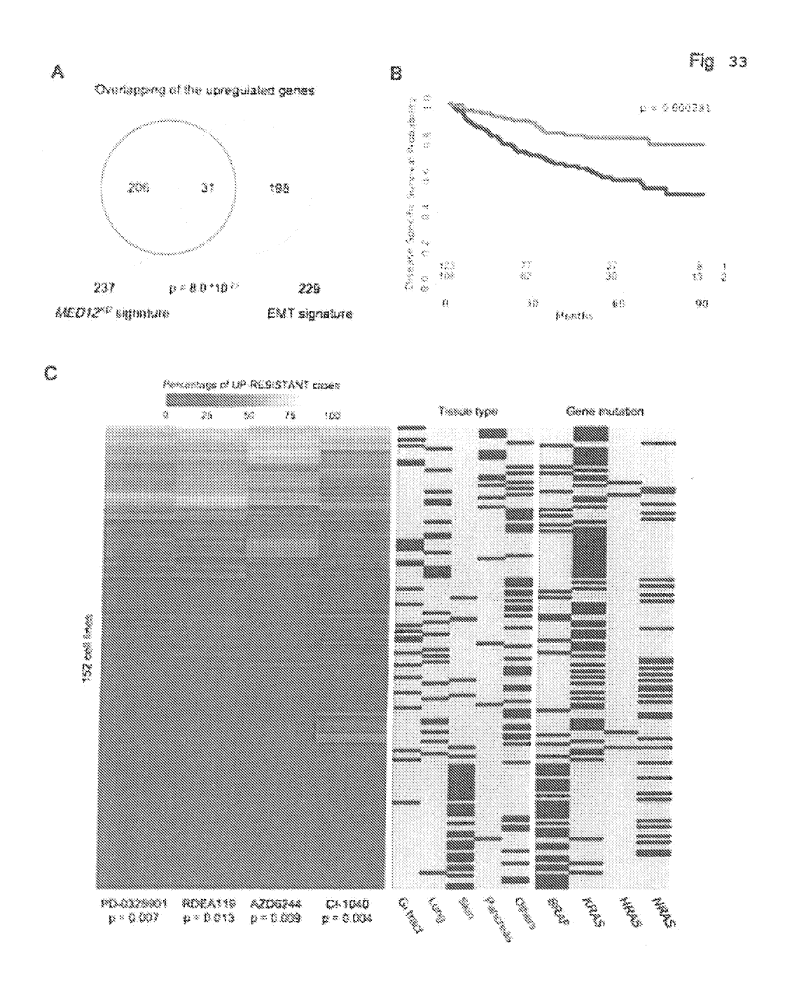

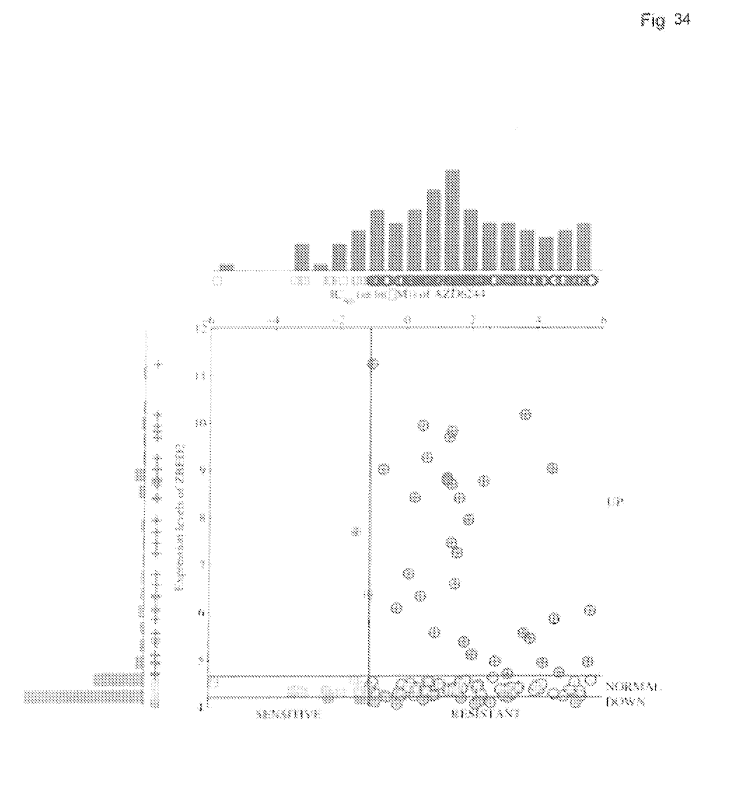

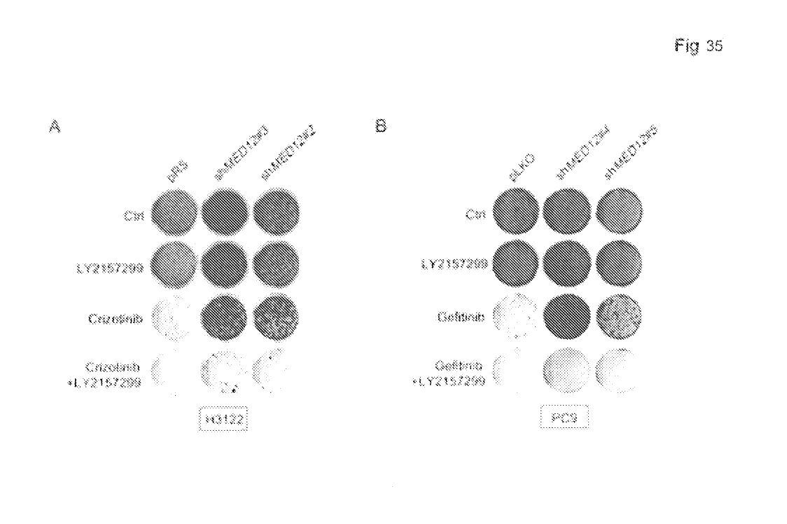

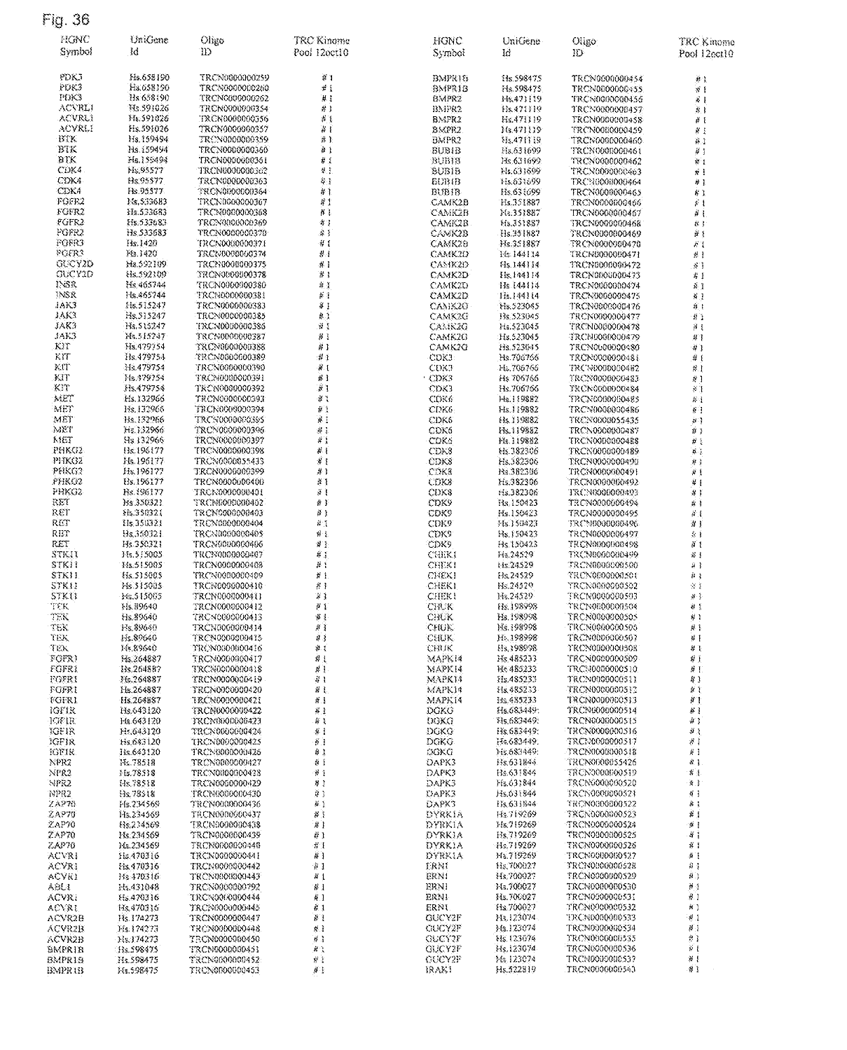

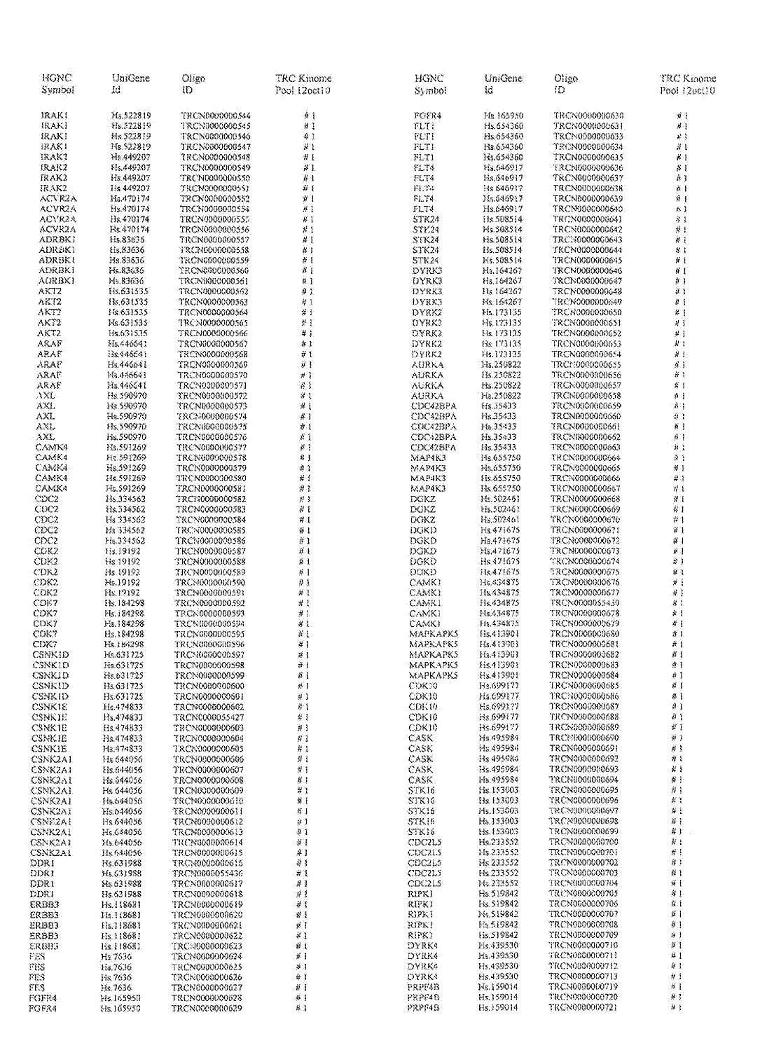

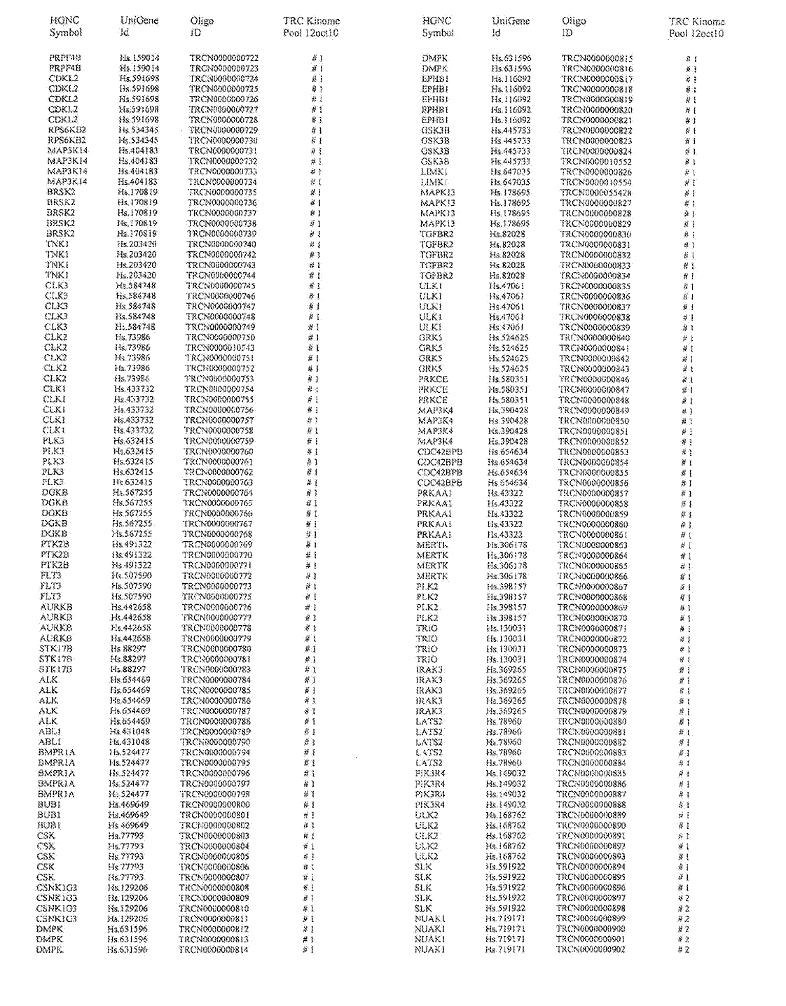

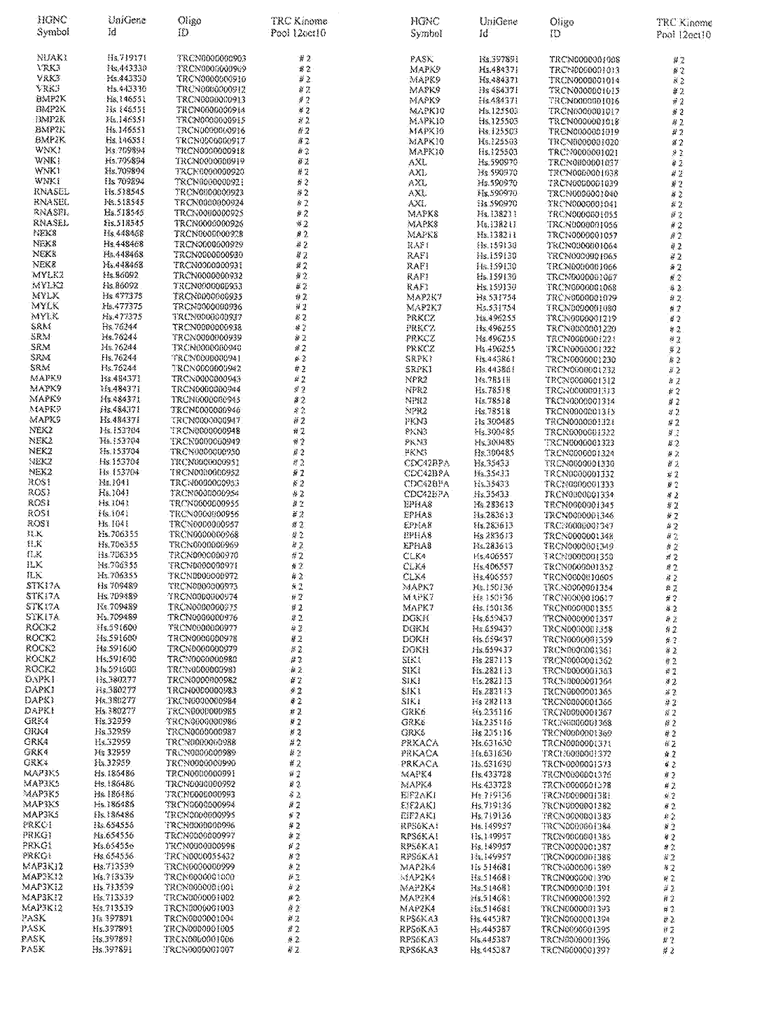

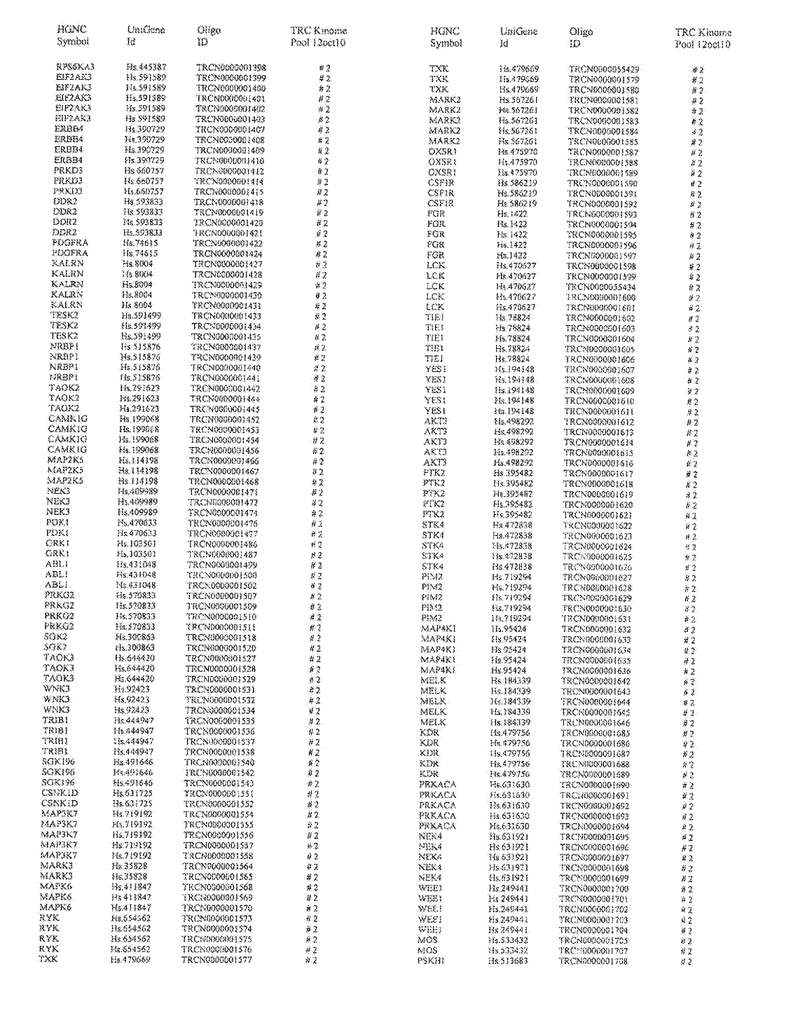

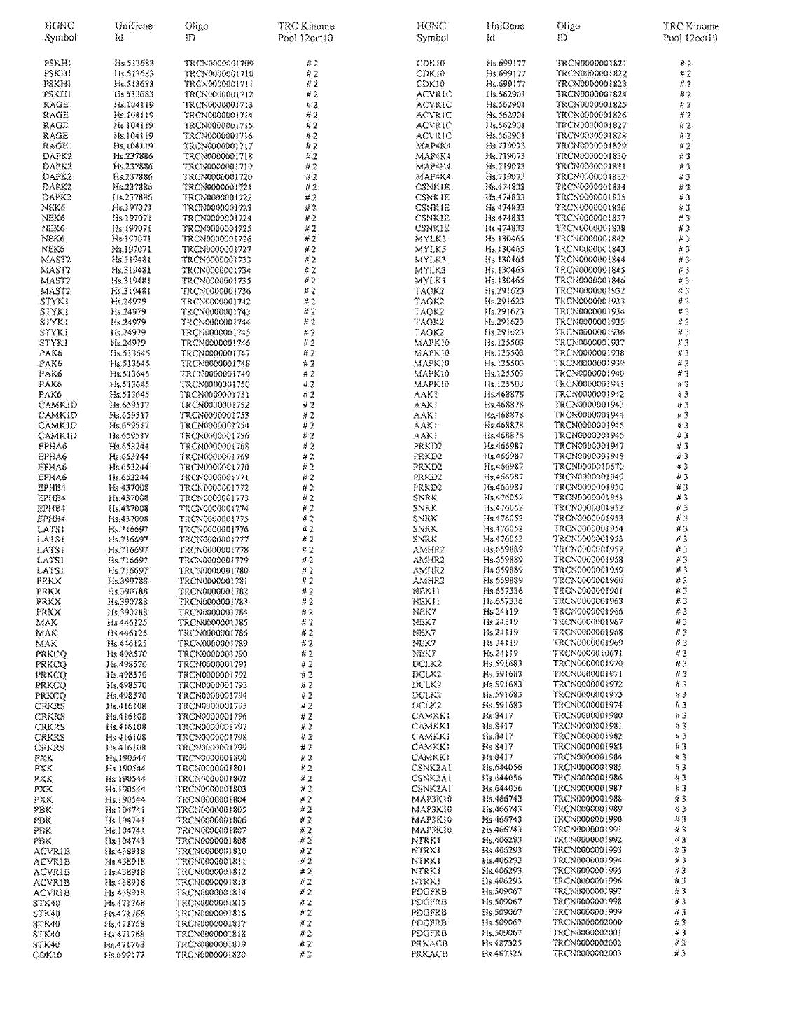

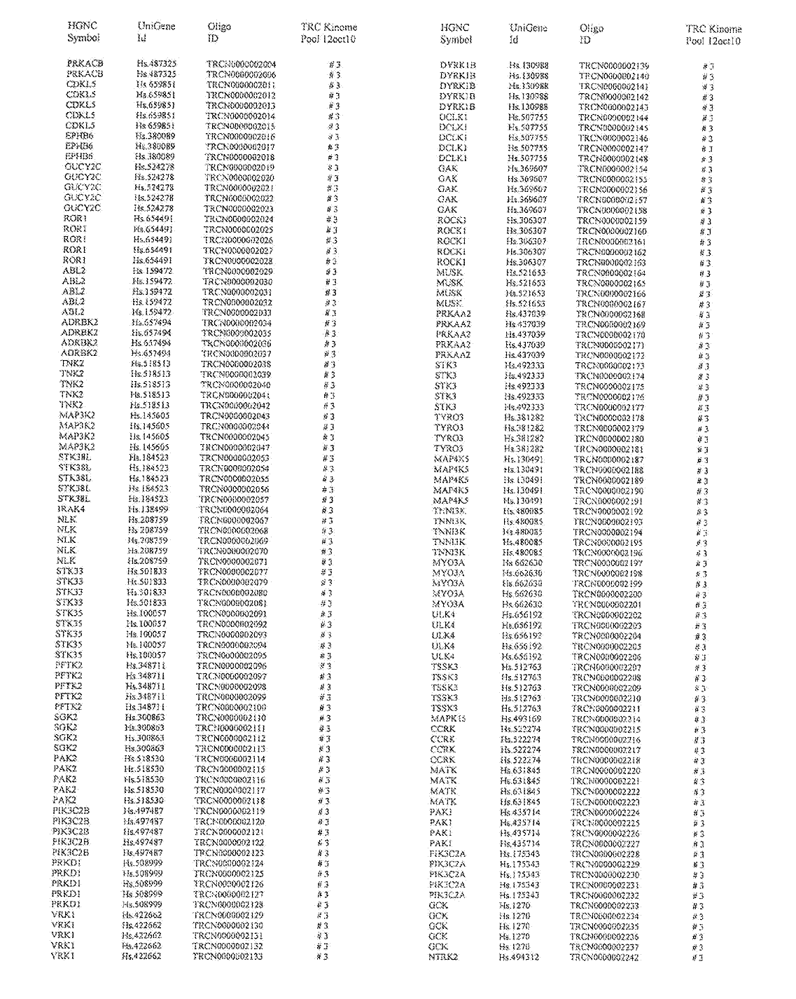

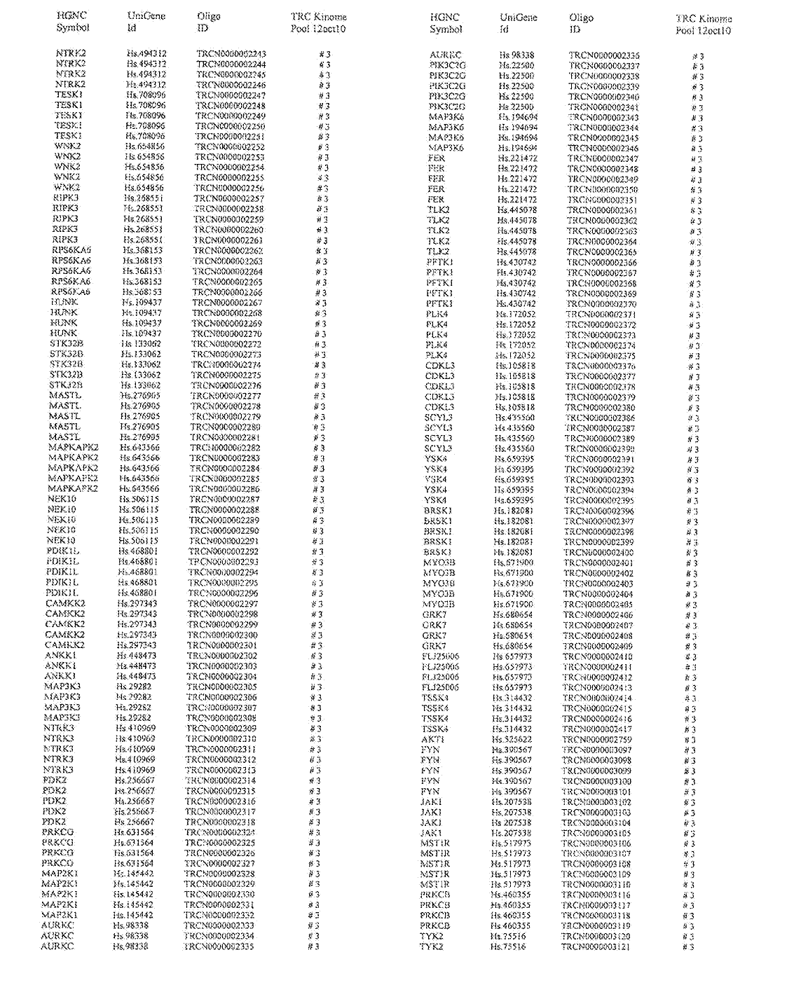

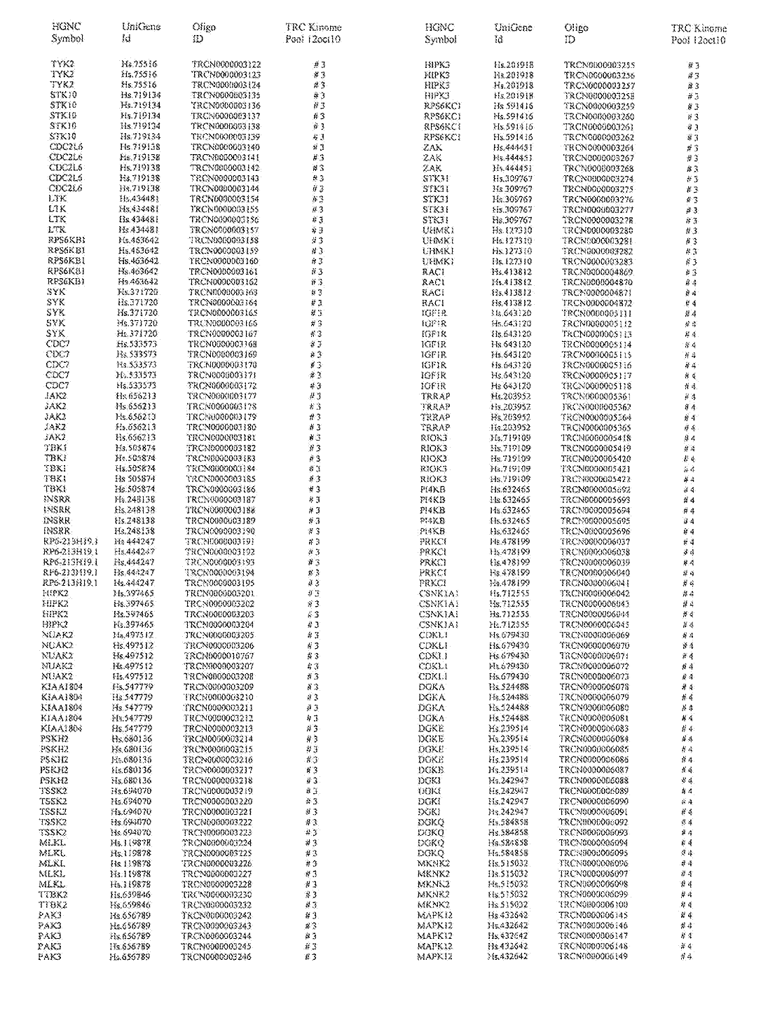

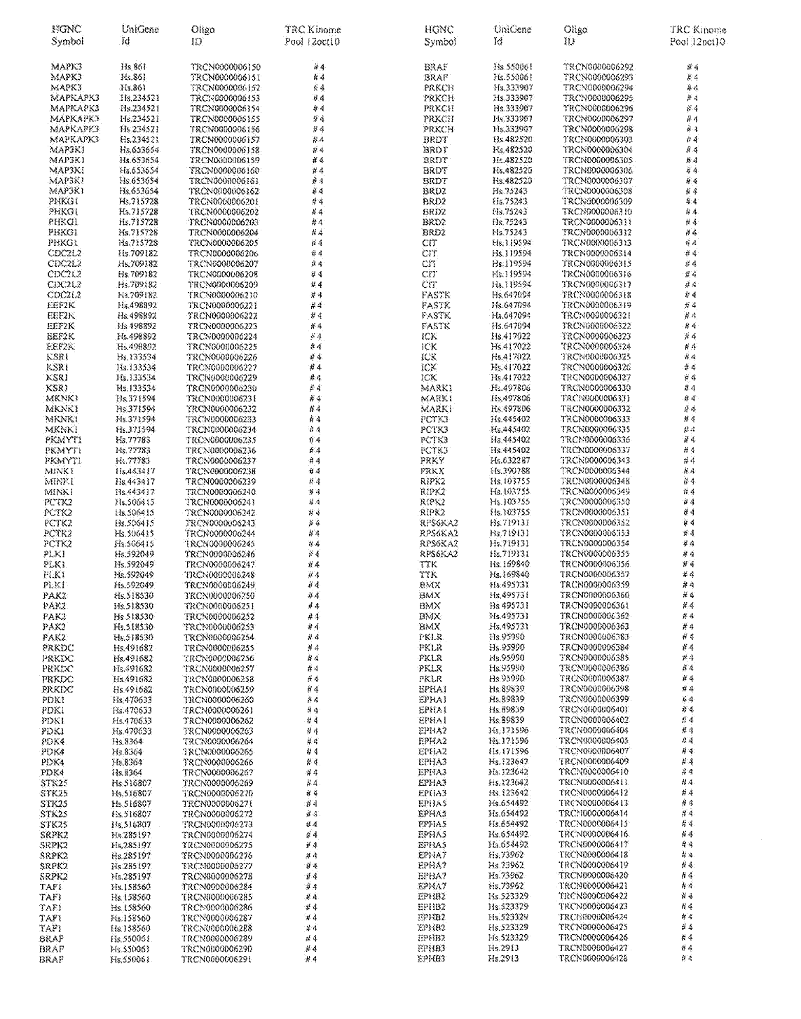

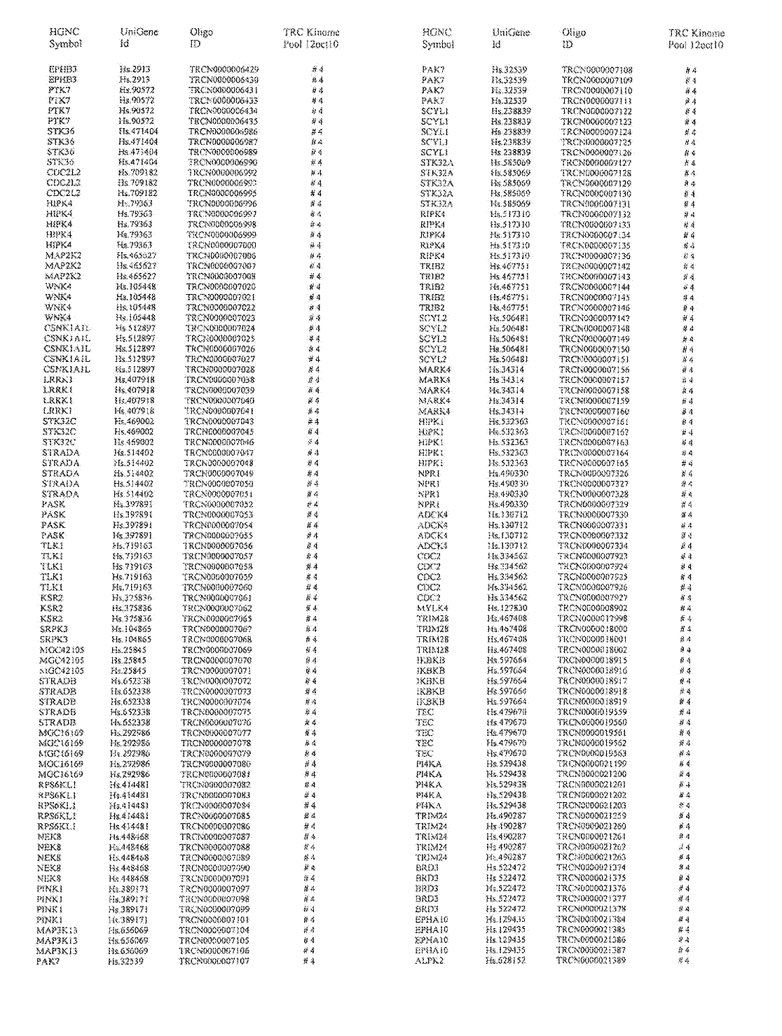

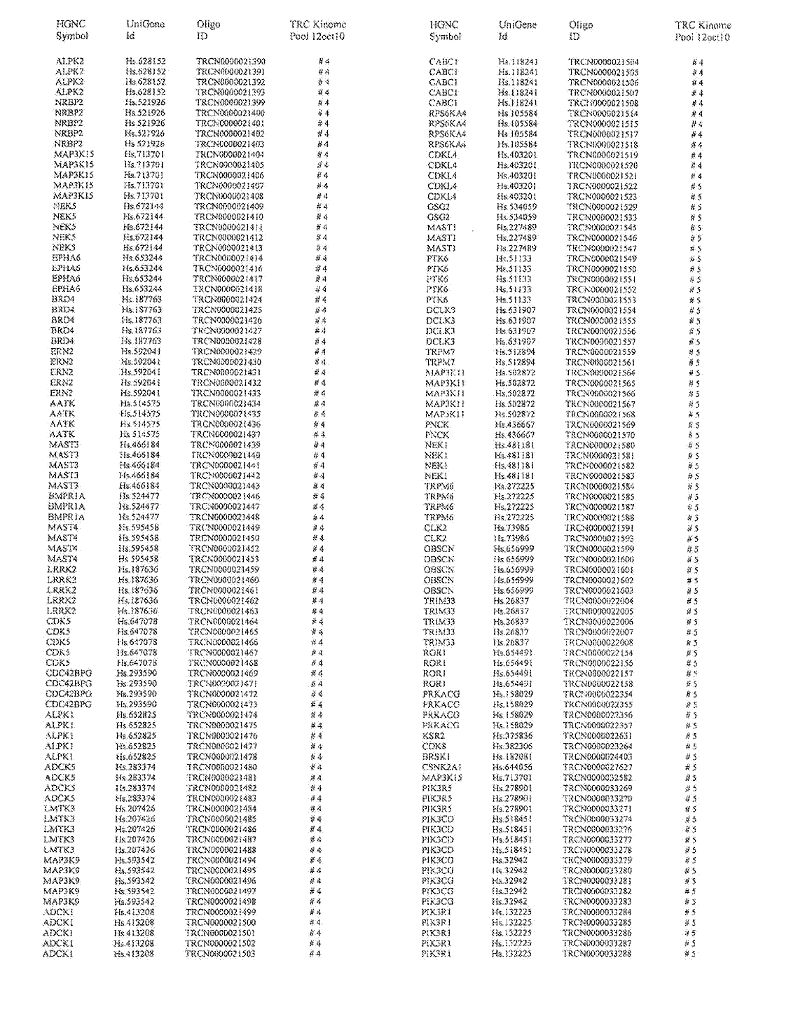

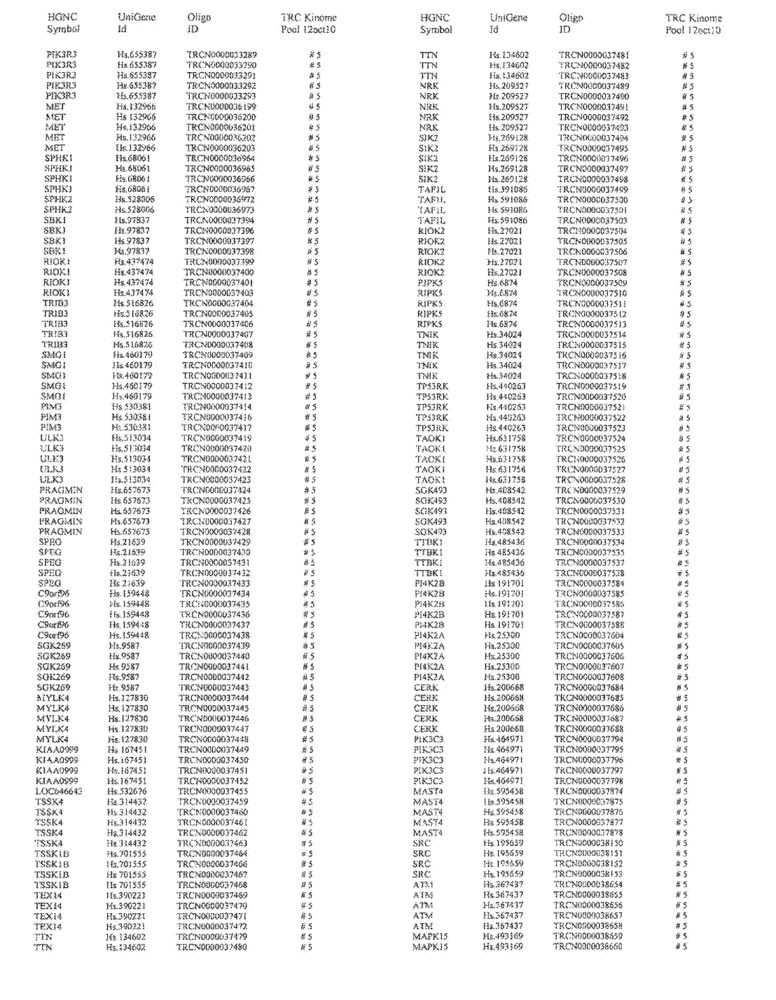

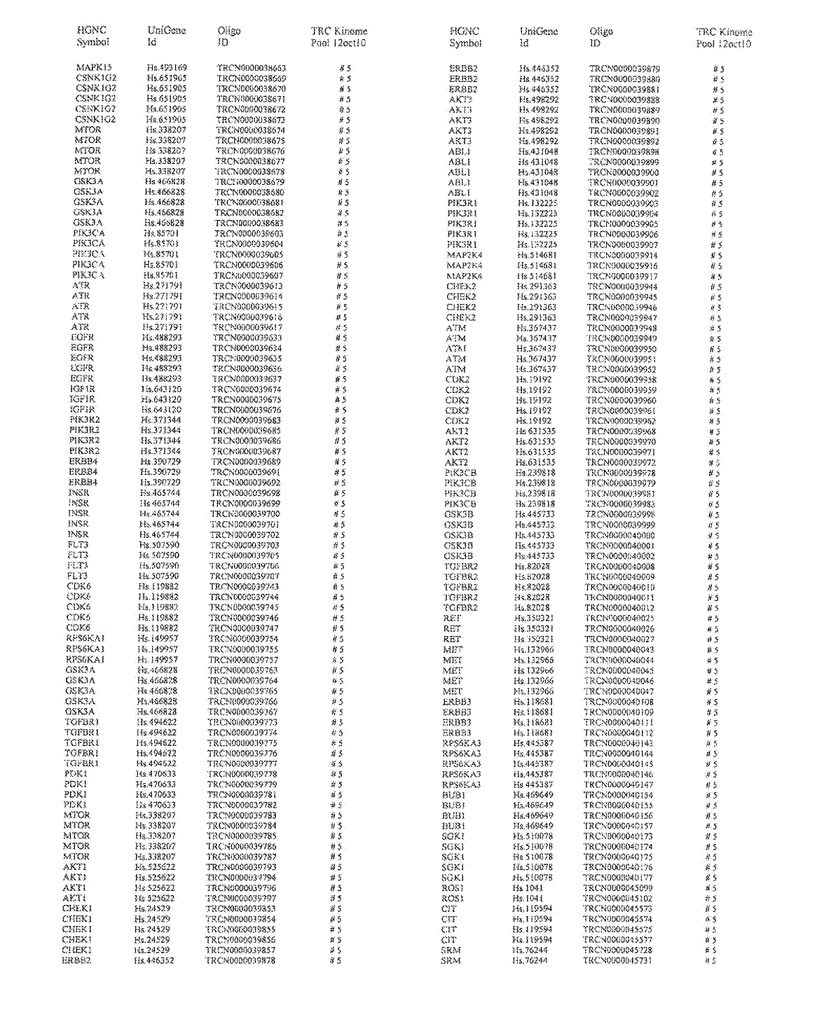

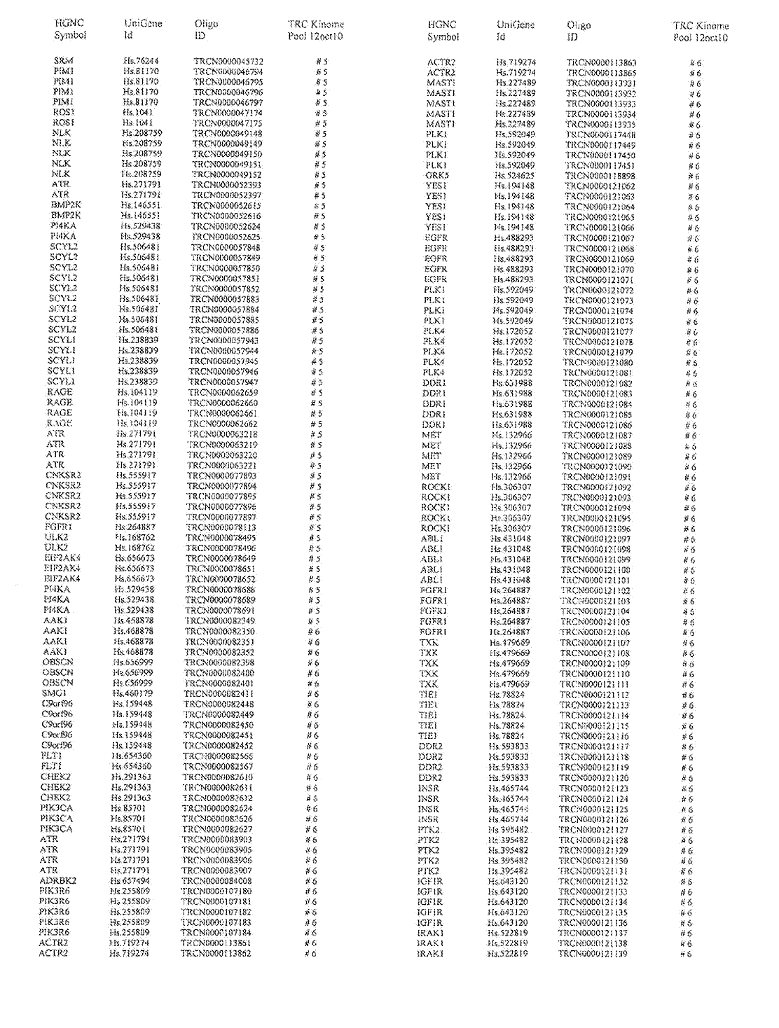

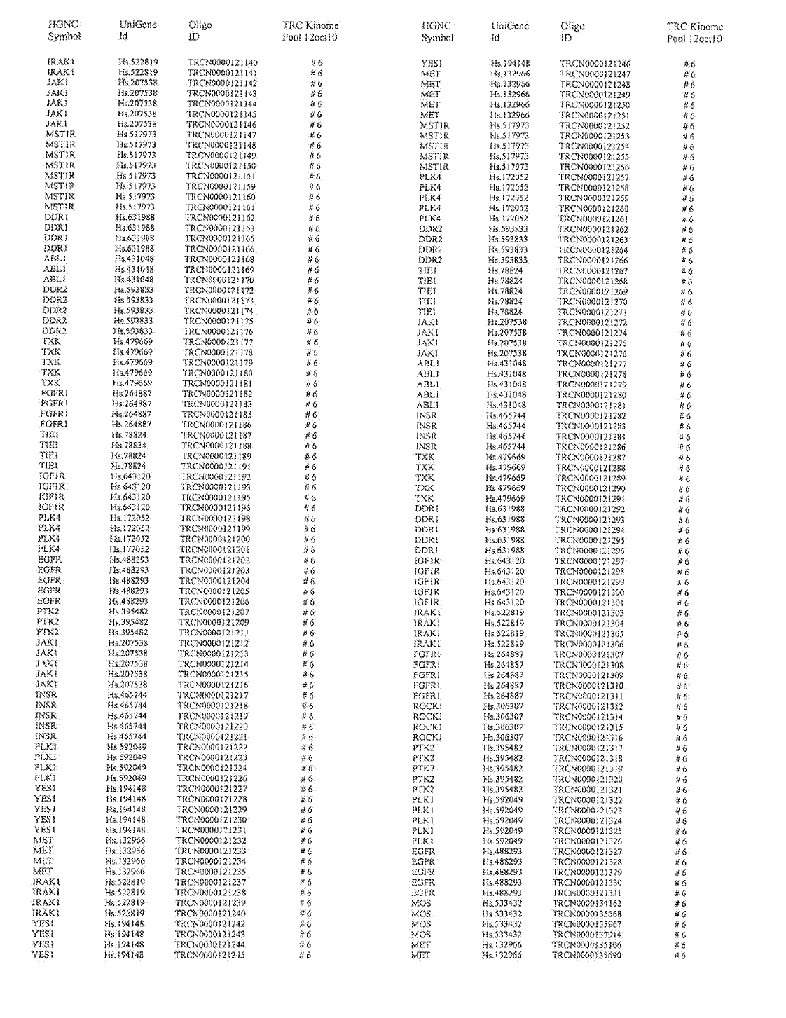

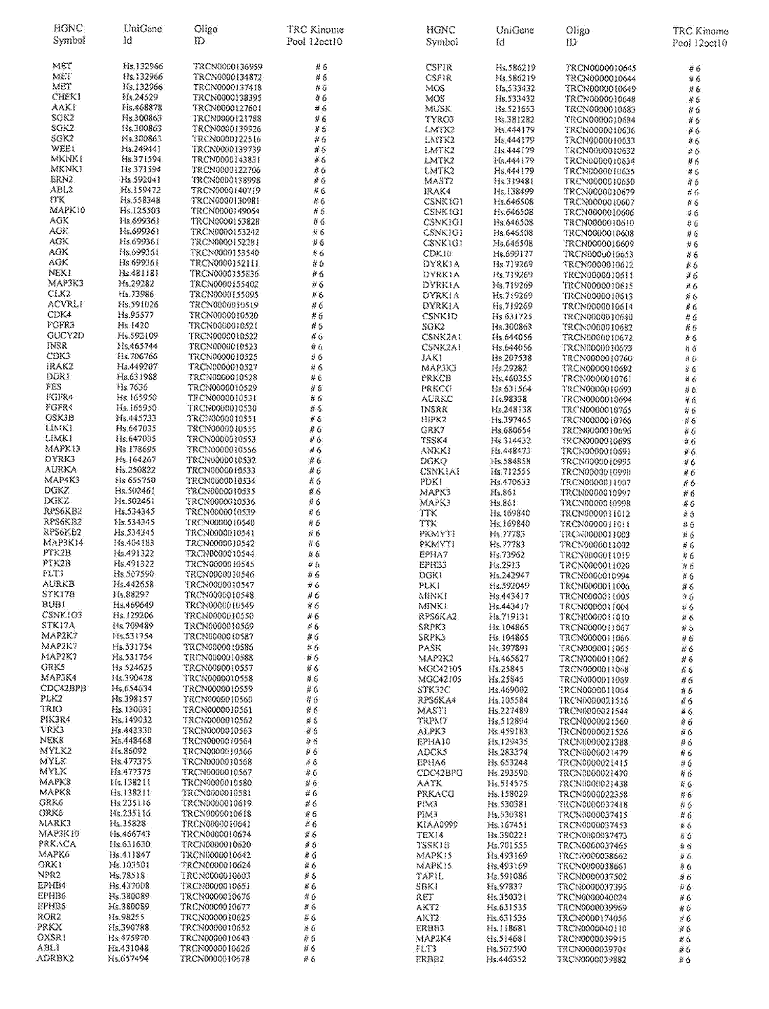

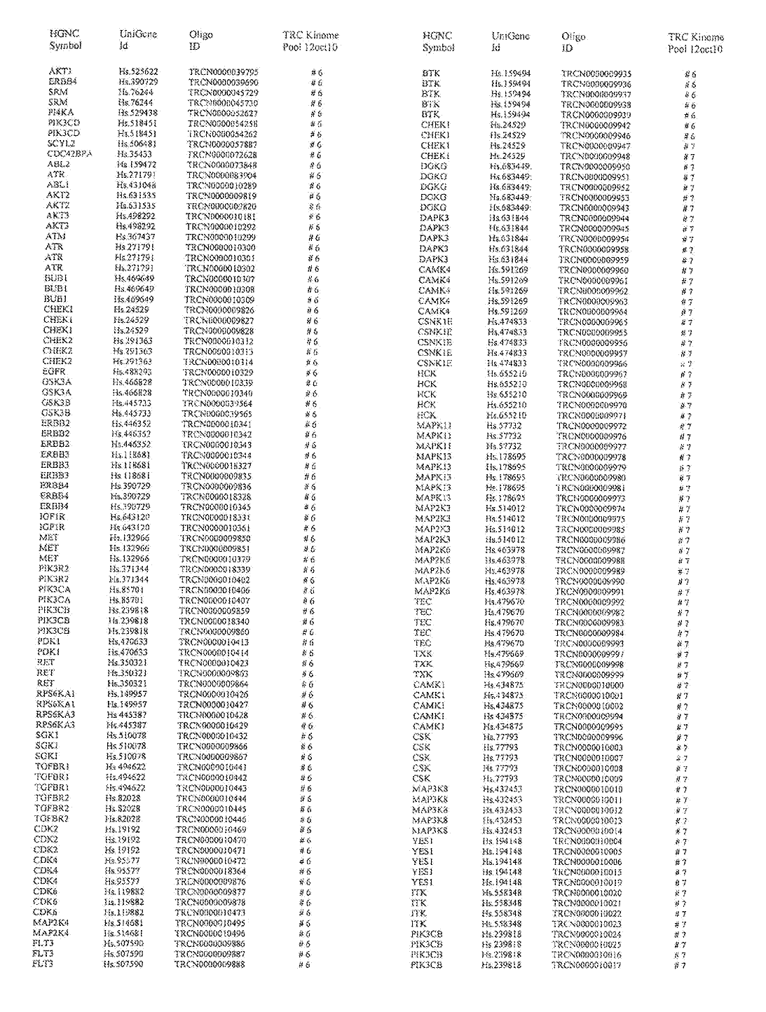

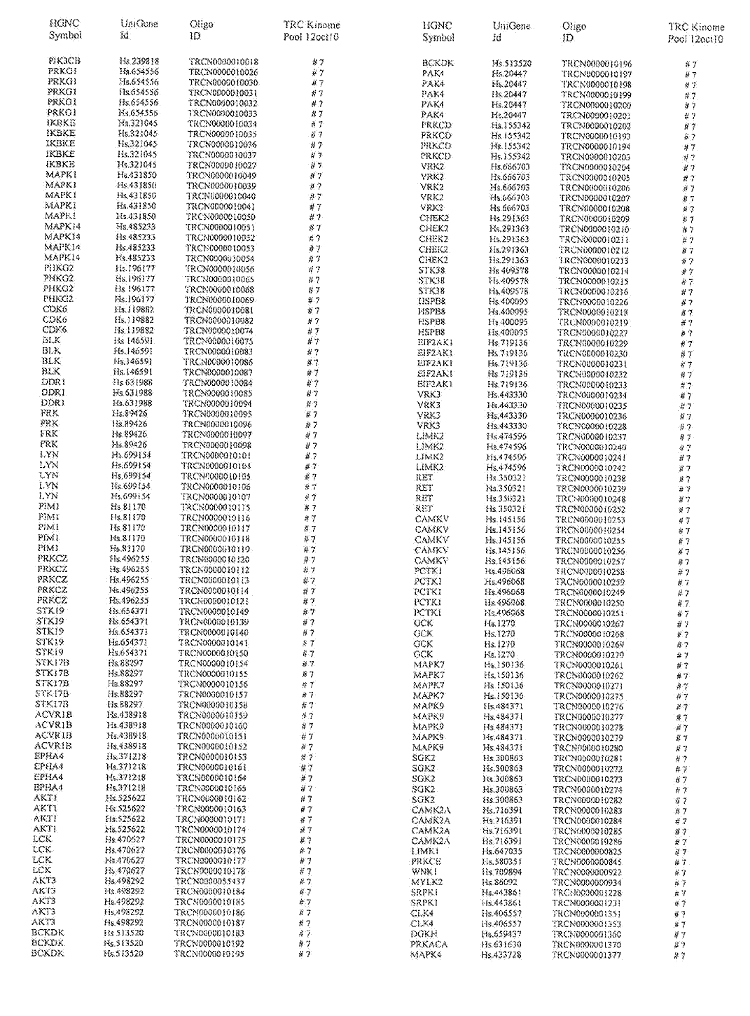

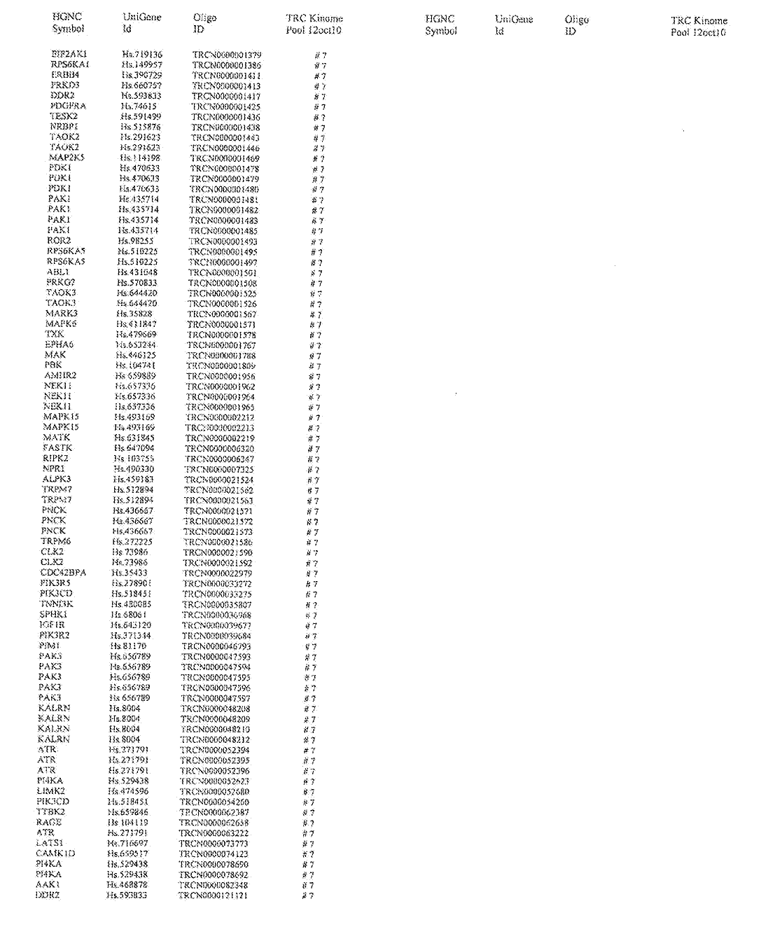

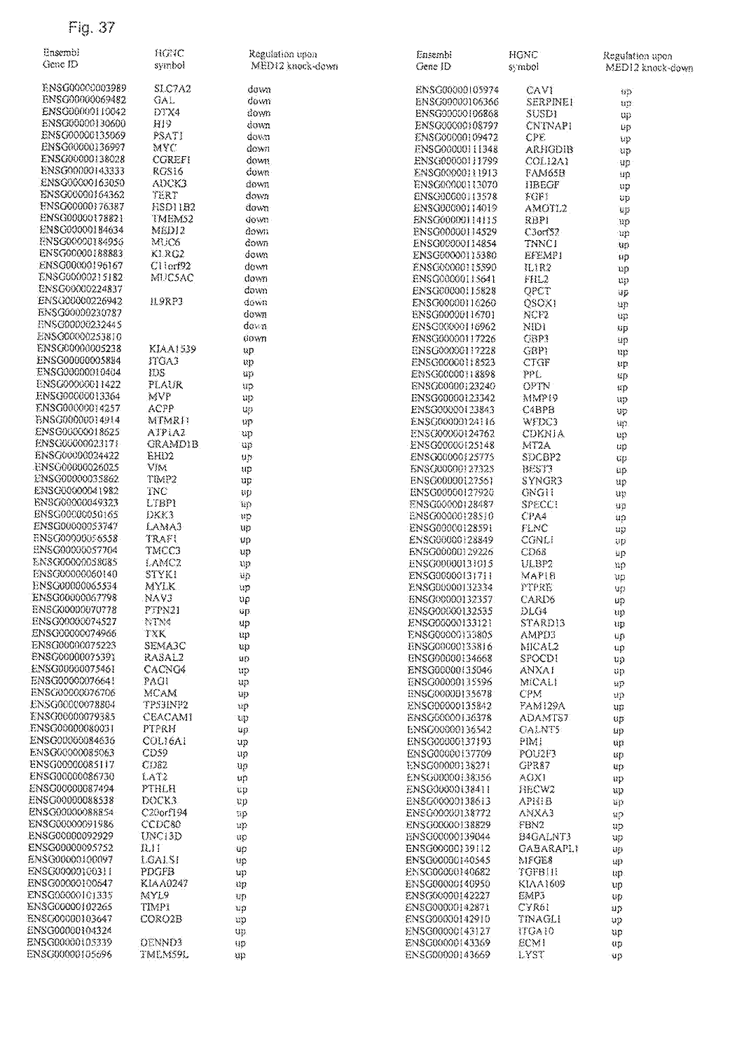

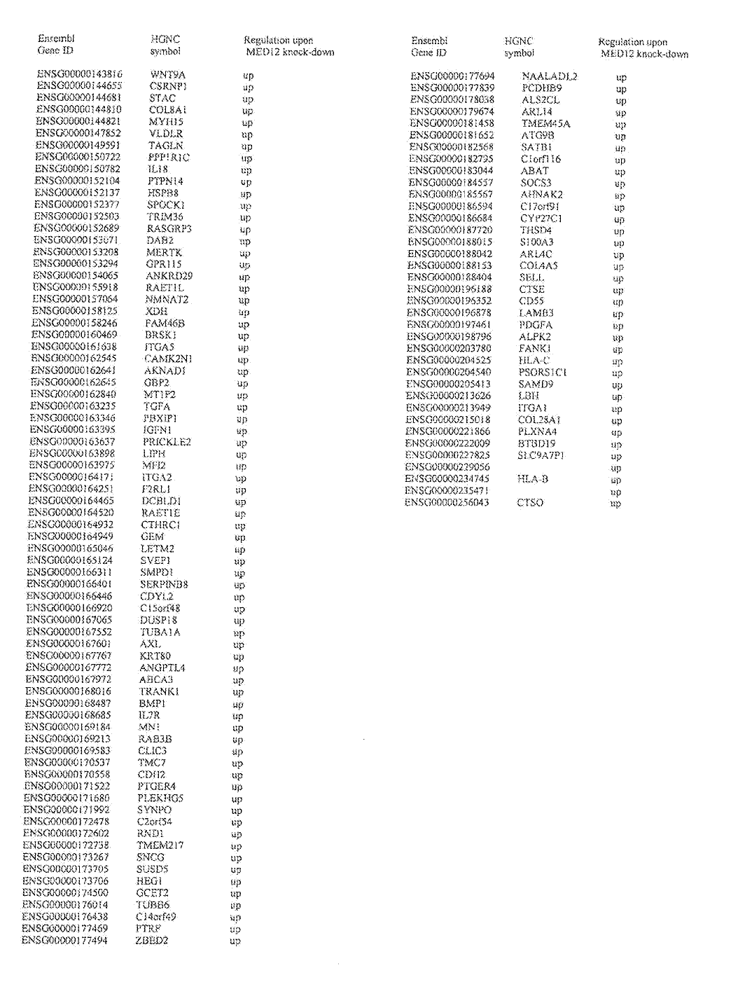

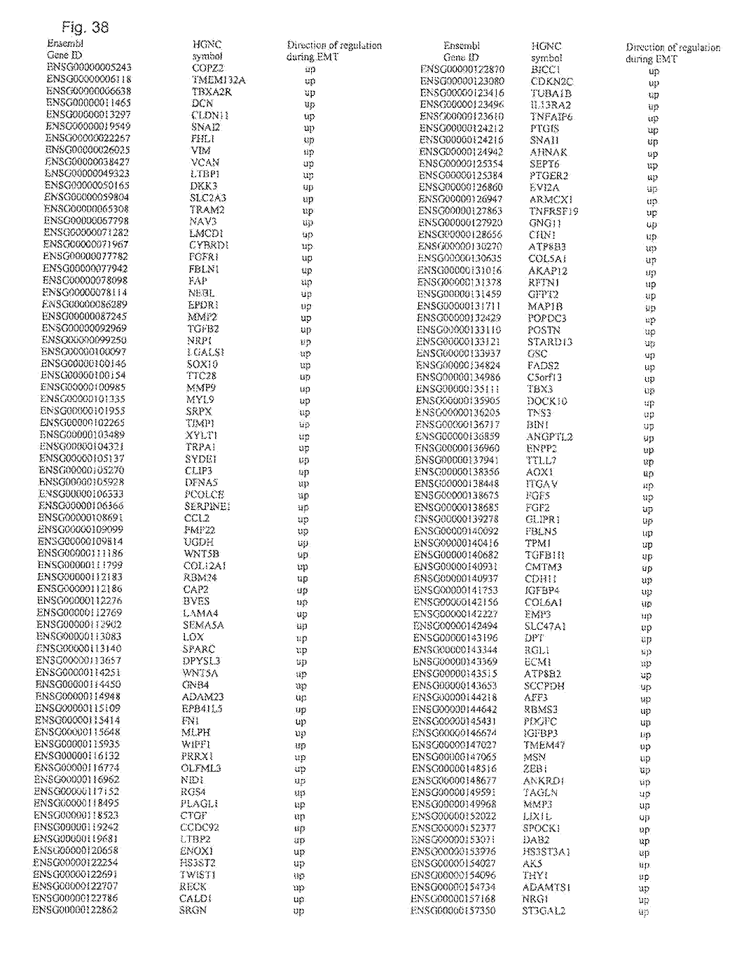

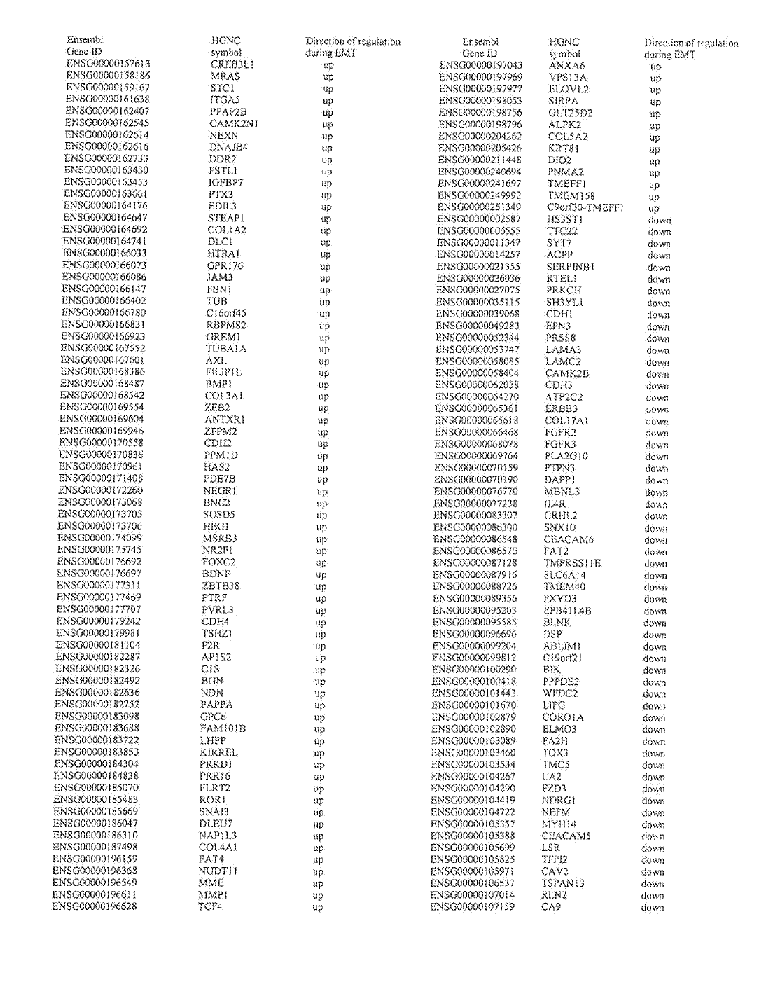

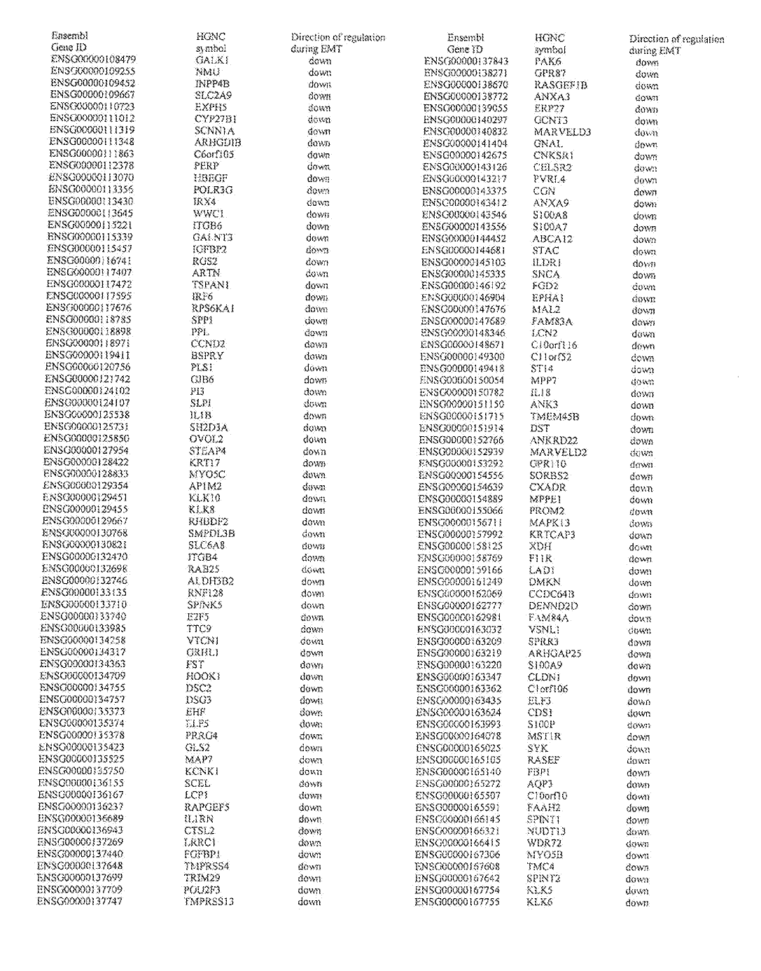

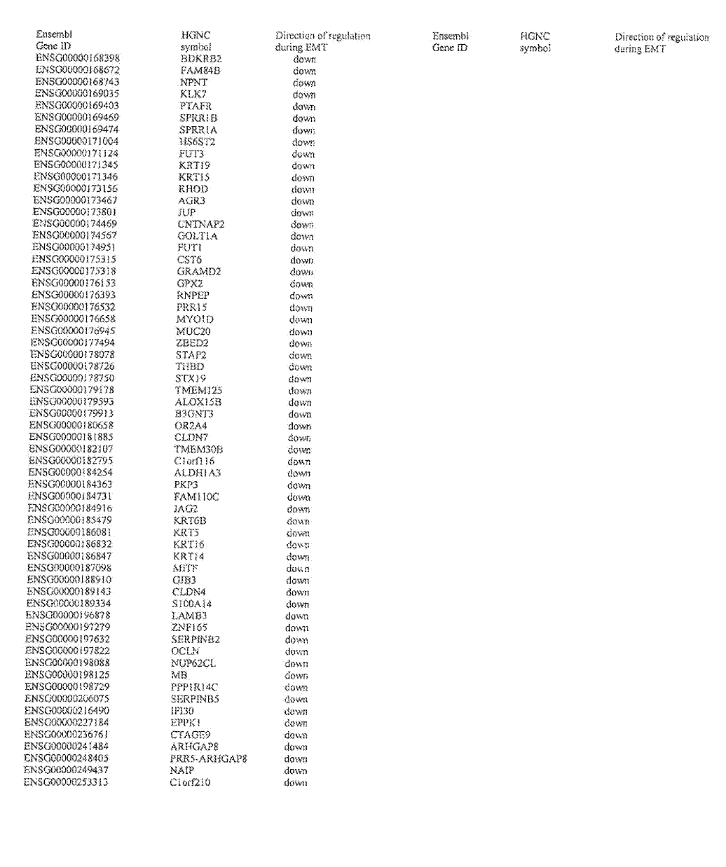

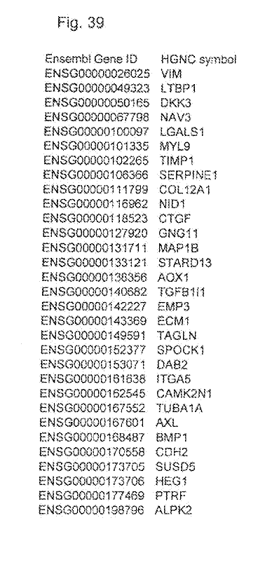

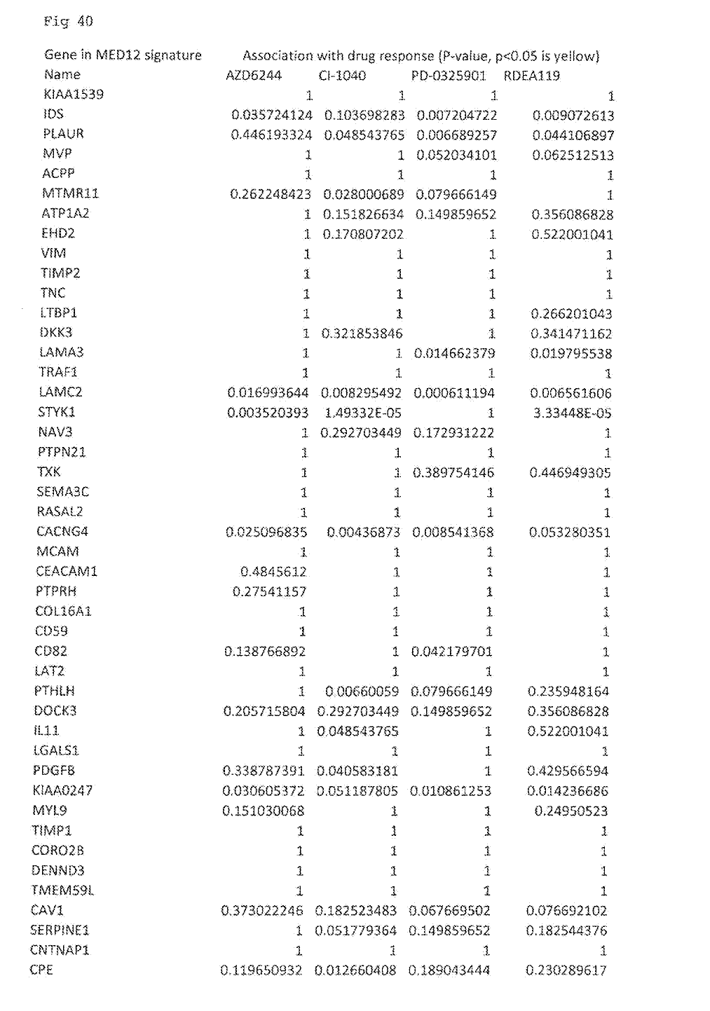

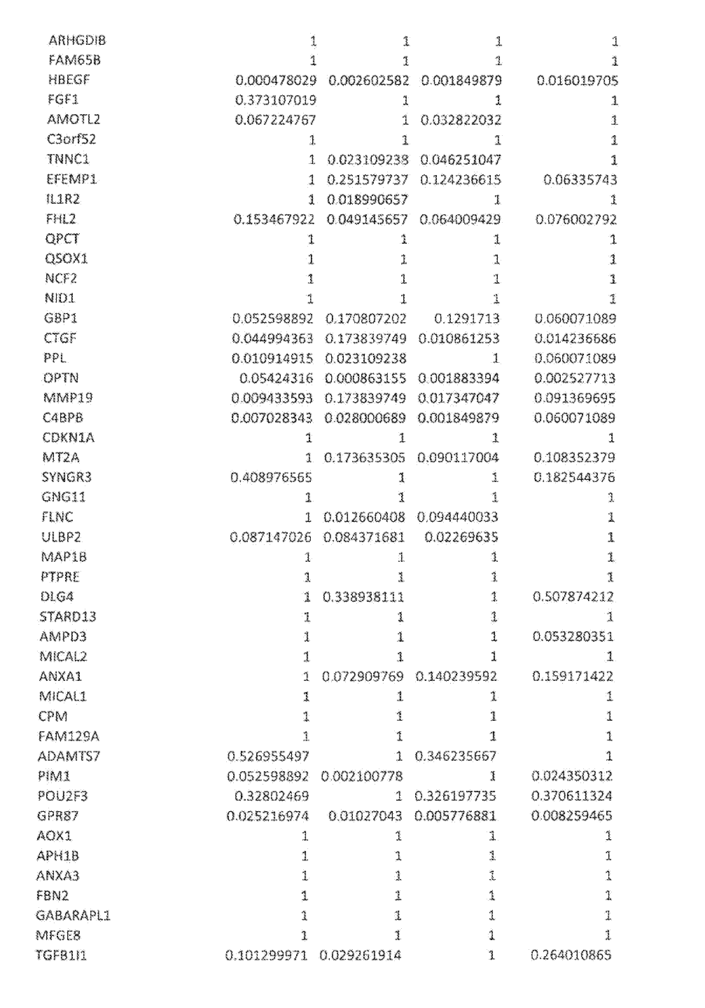

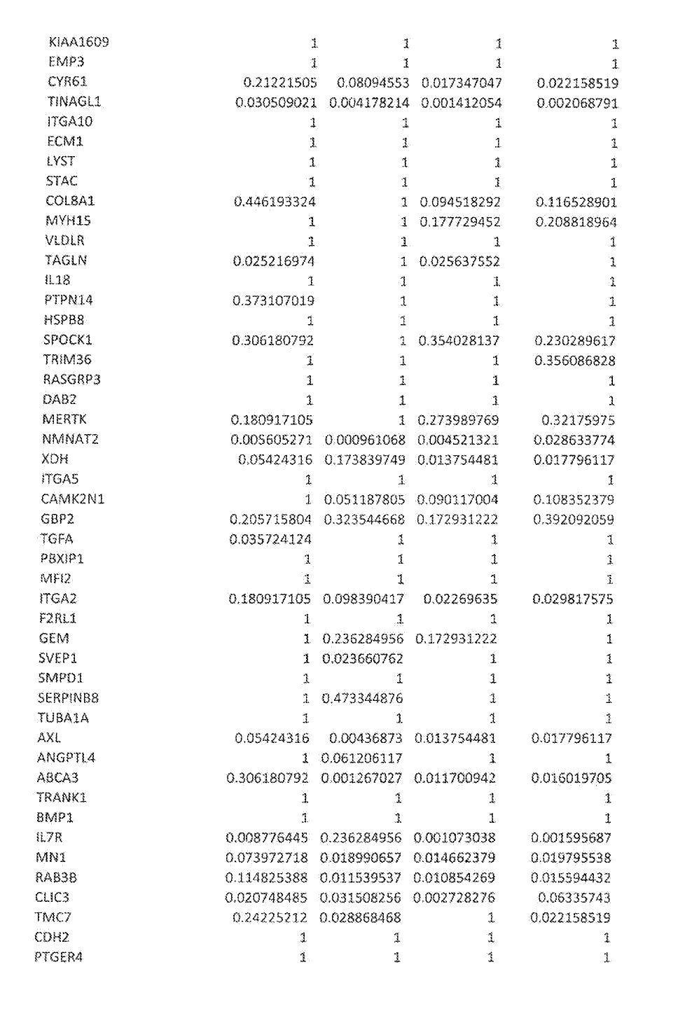

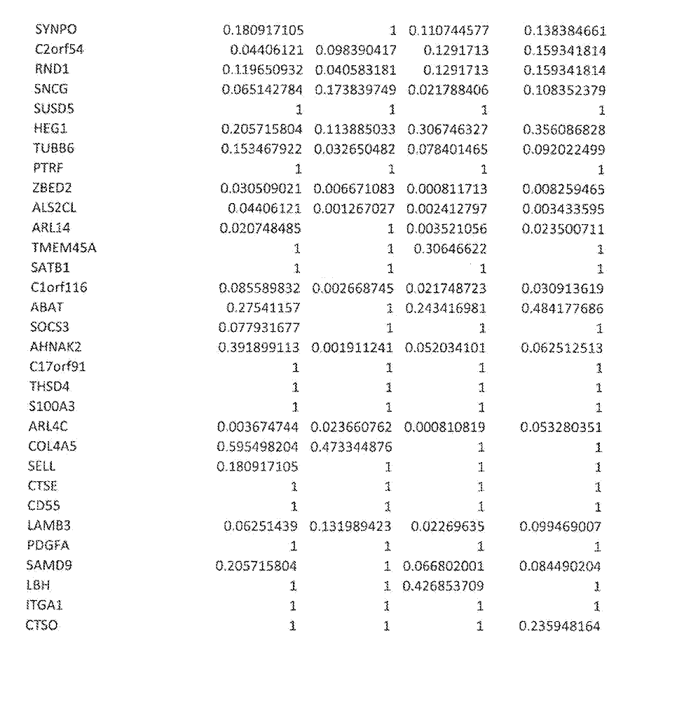

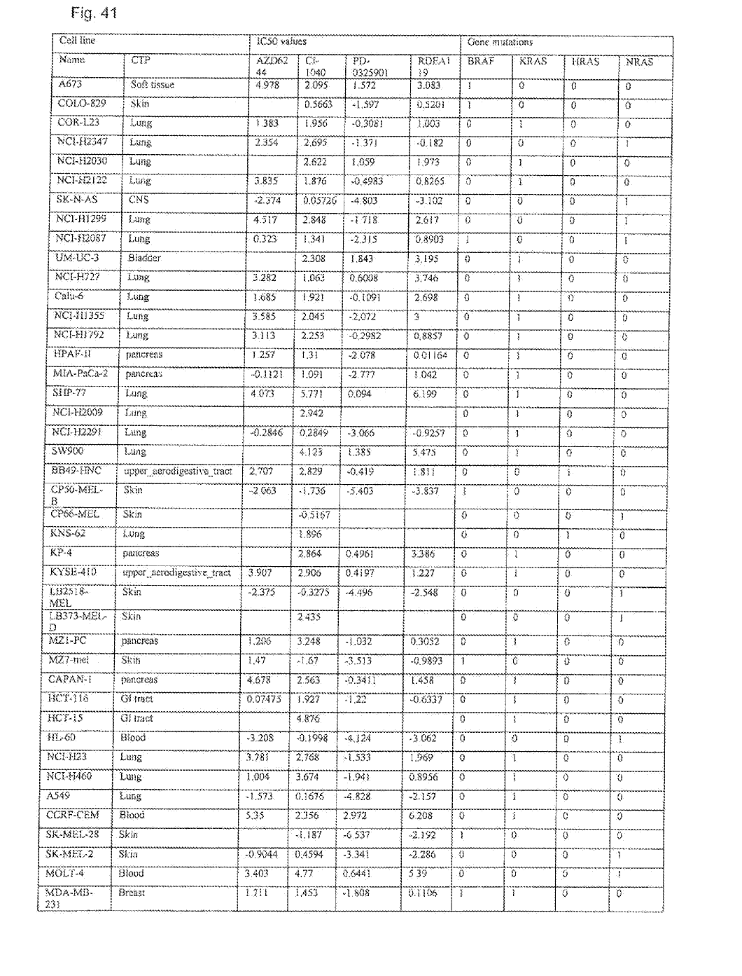

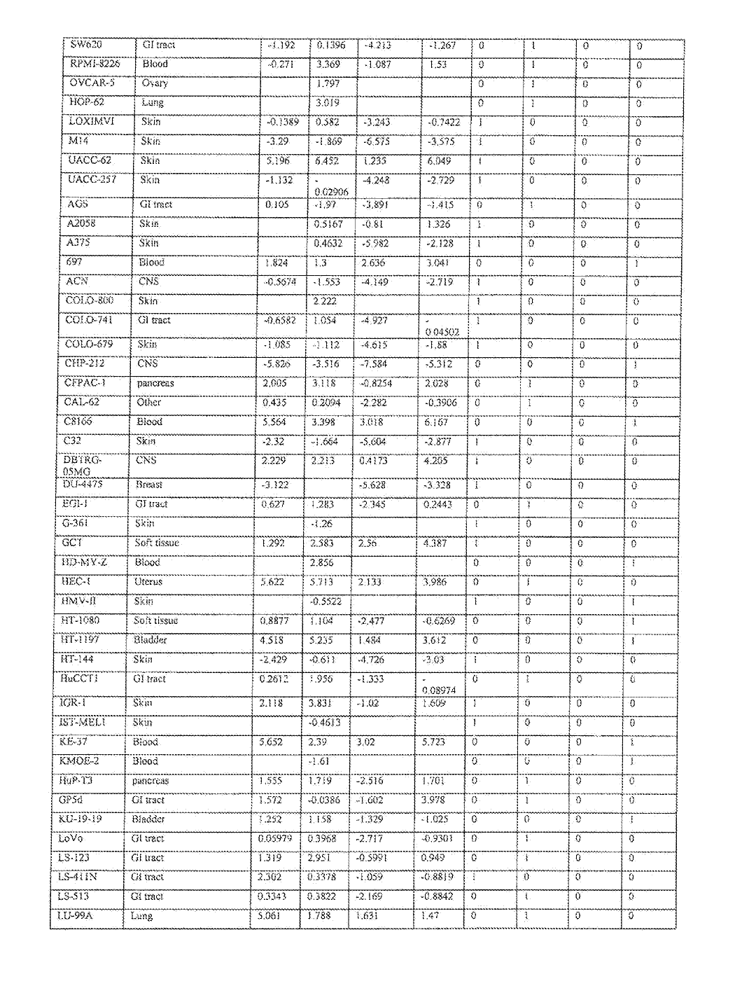

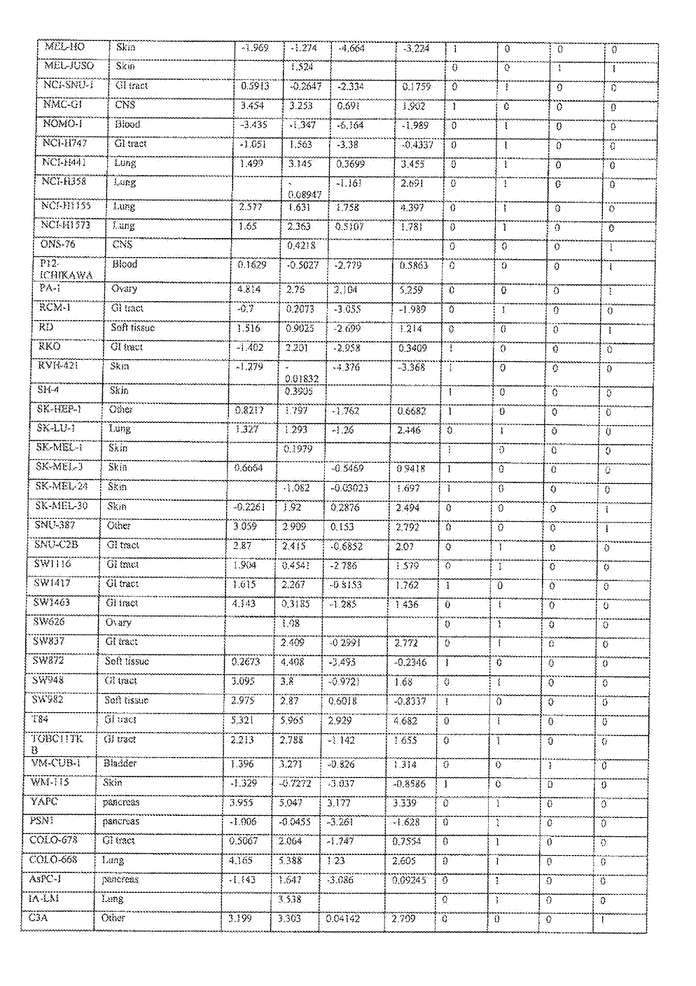

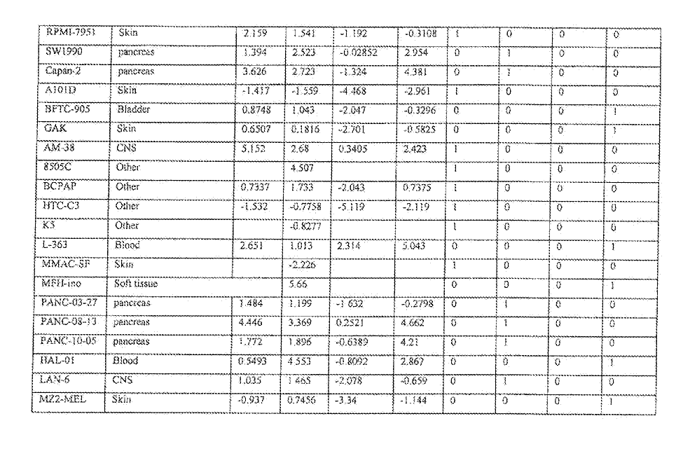

Applicants' data indicate that MED12 suppression also induces an EMT-like phenotype, as judged by the upregulation of the mesenchymal markers Vimentin and N-cadherin (FIGS. 29 and 30) and the general overlap between genes that are regulated by MED12^{KD} and known EMT signature genes (FIG. 33A). Applicants' data are consistent with the findings of others, who also witnessed resistance to EGFR inhibitors in cell lines undergoing EMT (Coldren et al., 2006; Frederick et al., 2007; Fuchs et al., 2008; Rho et al., 2009; Thomson et al., 2005; Yao et al., 2010). In the clinic, EMT transformation was also seen in 3 out of 7 NSCLC patients who developed resistance to EGFR TKIs and did not have one of the well-established secondary EGFR mutations causing drug resistance (Sequist et al., 2011). In some embodiments, such patients have acquired EMT as a result of MED12 loss. For example, MED12 was recently shown to be mutated in some 70% of uterine leiomyomas (Makinen et al., 2011). Applicants note that these mutations are highly clustered in the second exon of MED12, raising the possibility that these mutations are not null alleles. Consistent with this, Applicants observe that MED12 suppression often confers a slow-growth phenotype to cancer cells and that near-complete suppression of MED12 is not tolerated by most cells (FIGS. 2F, 17C, 17G and data not shown). Thus, in some embodiments, suppression of MED12 may not confer a selective advantage in the absence of drug, but may only become a benefit to the cancer cells when undergoing drug selection pressure. Consistent with this, Applicants observed that PC9 NSCLC, A375 melanoma and Huh-7 HCC cells are growth-inhibited by MED12^{KD}, but this turns into a proliferative advantage when exposed to EGFR, BRAF or MEK inhibitors or the multikinase inhibitor sorafenib (FIGS. 2F, 17C and 17G). Therefore, in some embodiments, MED12 suppression may not be a marker of intrinsic drug resistance as its constitutive suppression could well be disadvantageous to the cancer cell, but it may be acquired during drug selection to resist the therapy. That cancer cells can transiently assume a reversible drug-tolerant state was recently shown by others (Sharma et al., 2010).

In certain embodiments, cancer cells that undergo an EMT-like process do so through suppression of MED12 expression. Investigation of this would require biopsies of tumors that have progressed following exposure to targeted therapies, which are very rare in today's clinical practice. Applicants' data show that the changes of gene expression triggered by MED12 suppression (through analysis of a set of MED12^{KD} signature genes) are prognostic for disease outcome in colon cancer (FIG. 33B) and predictive for responses to MEK inhibitors in a large and heterogeneous cell line panel (FIG. 33C). In both of these studies, the mRNA levels of MED12 alone did not predict prognosis or drug responses (data not shown). This may be because MED12 protein levels are primarily regulated at a post-transcriptional level in tumors or because of alterations in MED12 activity as a result of mutation, as seen in leiomyomas (Makinen et al., 2011). Nevertheless, it is clear from Applicants' studies that MED12 suppression triggers activation of TGFβ signaling in tumors of lung, skin, liver and colon and results in an EMT-like phenotype associated with drug resistance. Applicants' data also demonstrate that inhibition of TGFβ signaling with small molecule drugs can reverse resistance to targeted cancer drugs (FIG. 35). Accordingly, in some embodiments, EMT arising during drug resistance development, as seen in NSCLC (Sequist et al., 2011), may be countered by combination with a TGFβ antagonist, a notion that can readily be tested in the clinic.

In certain embodiments, identification of a reduced expression of a MEDIATOR complex and/or SWI/SNF complex gene in one or more cancer cells of a patient is indicative that the one or more cancer cells will be resistant to treatment by a compound or class of compounds, such as one or more receptor tyrosine kinase inhibitor compounds. Examples of RTK inhibitor compounds that cells expressing a reduced level of a MEDIATOR complex and/or SWI/SNF complex gene may be resistant to include gefitinib, erlotinib, EKB-569, lapatinib, CI-1033, cetuximab, panitumumab, PKI-166, AEE788, sunitinib, sorafenib, dasatinib, nilotinib, pazopanib, vandetaniv, cediranib, afatinib, motesanib, CUDC-101, and imatinib mesylate. Other RTK inhibitors that cells expressing a reduced level of a MEDIATOR complex and/or SWI/SNF complex gene may be resistant to include the Alk-1 inhibitors crizotinib, ASP-3026, LDK378, AF802, and CEP37440.

In certain embodiments, identification of a reduced expression of a MEDIATOR complex and/or SWI/SNF complex gene in one or more cancer cells of a patient is indicative that the one or more cancer cells will be resistant to treatment by one or more ERK activation inhibitor compounds. Examples of ERK activation inhibitor compounds that cells expressing a reduced level of a MEDIATOR complex and/or SWI/SNF complex gene may be resistant to include compounds that inhibit the activity of a signaling protein upstream of ERK. Examples of signaling proteins upstream of ERK include MEK1, MEK2, A-RAF, B-RAF, RAF1, MOS, RTKs, and G-protein-coupled receptors. In certain embodiments, the compound that inhibits the activity of a signaling protein upstream of ERK inhibits a direct activator of ERK. Examples of direct ERK activators include MEK1 and MEK2. Examples of MEK inhibitors include CKI-27, RO-4987655, RO-5126766, PD-0325901, WX-554, AZD-8330, G-573, RG-7167, SF-2626, GDC-0623, RO-5068760, and AD-GL0001. In other embodiments, the compound that inhibits the activity of a signaling protein upstream of ERK inhibits an indirect activator of ERK. Examples of indirect ERK activators include A-RAF, B-RAF, RAF1RAF1, MOS, RTKs, and G-protein-coupled receptors. See, e.g., Roux, P P, Blenis, J “ERK and p38 MAPK-activated protein kinases: a family of protein kinases with diverse biological functions” Microbiol Mol Biol Rev. (2004) 68(2):320-44. Examples of B-RAF inhibitors include CEP-32496, vemurafenib, GSK-2118436, ARQ-736, RG-7256, XL-281, DCC-2036, GDC-0879, AZ628, and antibody fragment EphB4/Raf inhibitors.

In some embodiments, an inhibitor inhibits the wild-type version of a protein, such as wild-type B-RAF. In other embodiments, an inhibitor inhibits a mutant form of a protein, such as mutant B-RAF (e.g., V600E). In yet other embodiments, an inhibitor inhibits both the wild-type and mutant form of a protein (e.g., both wild-type B-RAF and B-RAF^{V600E}).

In certain embodiments, identification of a reduced expression of a MEDIATOR complex and/or SWI/SNF complex gene in one or more cancer cells of a patient is indicative that the one or more cancer cells will be resistant to treatment by one or more compounds that are activators of one or more proteins that inactivate ERK. Examples of protein inactivators of ERK include phosphatases, such as the indirect inactivator of ERK, protein phosphatase 5 (PP5), which inactivates the ERK upstream activator, RAF1, by dephosphorylation.

In certain embodiments, the prognostic methods and compositions of the instant invention predict resistance to anticancer treatment to a combination of chemotherapeutic agents, wherein the at least two chemotherapeutic agents are administered at the same time and/or sequentially. In further embodiments, the invention provides methods wherein a measurement of reduced expression of a MEDIATOR complex and/or SWI/SNF complex and/or RAS-GAP gene in one or more cancer cells of a tumor of a patient identifies the tumor as one that may be resistant to treatment by a combination of at least two ERK activation inhibitors. In other embodiments, the tumor is one that may be resistant to treatment by a combination of at least two compounds that activate one or more proteins upstream of ERK that inactivates ERK signaling.

In some embodiments, activation of the TGF-β (transforming grow factor beta) pathway rescues ERK activation in, for example, a cancer cell. Accordingly, in some embodiments, the prognostic methods and compositions of the instant invention provide methods and compositions wherein a measurement of reduced expression of a MEDIATOR complex and/or SWI/SNF complex and/or RAS-GAP gene in one or more cancer cells of a tumor of a patient identifies the tumor as one that may benefit from treatment with an inhibitor of the TGFβ pathway (e.g., a TGFβ inhibitor and/or inhibitor of one or more downstream signaling proteins in the TGF-β pathway) in combination with one or more ERK activation inhibitors. In other embodiments, the prognostic methods and compositions of the instant invention provide methods and compositions wherein a measurement of reduced expression of a MEDIATOR complex and/or SWI/SNF complex and/or RAS-GAP gene in one or more cancer cells of a tumor of a patient identifies the tumor as one that may benefit from treatment with an inhibitor of the TGF-β pathway in combination with one or more compounds that activate one or more proteins upstream of ERK that inactivates ERK signaling. In certain embodiments, the inhibitor of ERK activation is an RTK inhibitor. In other embodiments, the inhibitor of ERK activation is a B-RAF inhibitor. In yet other embodiments, the inhibitor of ERK activation is a MEK inhibitor. In still other embodiments, the inhibitor of ERK activation is a RAS inhibitor.

In other embodiments, the prognostic methods and compositions of the instant invention provide methods and compositions wherein a measurement of increased expression of a TGFβ pathway gene in one or more cancer cells of a tumor of a patient identifies the tumor as one that may benefit from treatment with an inhibitor of the TGFβ pathway (e.g., a TGFβ inhibitor and/or inhibitor of one or more downstream signaling proteins in the TGFβ pathway) in combination with one or more ERK activation inhibitors. In certain embodiments, the patient is one in need of treatment with an ERK activation inhibitor. In other embodiments, the patient is one in need of treatment with an inhibitor of a TGFβ pathway gene or protein. In other embodiments, the prognostic methods and compositions of the instant invention provide methods and compositions wherein a measurement of increased expression of a TGFβ pathway gene in one or more cancer cells of a tumor of a patient identifies the tumor as one that may benefit from treatment with an inhibitor of the TGFβ pathway in combination with one or more compounds that activate one or more proteins upstream of ERK that inactivates ERK signaling. In certain embodiments, the inhibitor of ERK activation is an RTK inhibitor. In other embodiments, the inhibitor of ERK activation is a B-RAF inhibitor. In yet other embodiments, the inhibitor of ERK activation is a MEK inhibitor. In still other embodiments, the inhibitor of ERK activation is a RAS inhibitor.

In other embodiments, the prognostic methods and compositions of the instant invention provide methods and compositions wherein a measurement of increased expression of a TGFβ pathway gene in one or more cancer cells of a patient indicates the patient may be resistant to anticancer treatment. In other embodiments, the prognostic methods and compositions of the instant invention provide methods and compositions wherein a measurement of an activating mutation in a TGFβ pathway gene in one or more cancer cells of a patient identifies the one or more cancer cells as cells that may be resistant to anticancer treatment.

In some embodiments, the invention provides methods and compositions for the treatment of primary and/or secondary resistance to one or more anticancer agents in a patient in need thereof, comprising administration of at least one inhibitor of the TGFβ pathway in combination with the one or more anticancer agents to which primary and/or secondary resistance in the patient has developed. For example, in some embodiments, the invention relates to a method of treating secondary resistance to an inhibitor of ERK activation in a patient in need thereof, comprising administering to the patient at least one inhibitor of the TGFβ pathway (e.g., a TGFβ inhibitor) in combination with the inhibitor of ERK activation.

In certain embodiments, the invention provides methods and compositions related to a method of treating cancer in a patient in need thereof, comprising administering to the patient an inhibitor of ERK activation in combination with an inhibitor of TGFβ pathway activation. In some embodiments, the patient is treated without determining whether the patient would be likely to be resistant to one or more of the ERK activation and/or TGFβ pathway activation inhibitors.

In some embodiments, the markers of the instant invention enable the detection of resistance to anticancer treatment in a patient in combination with one or more known markers of hypersensitivity to a chemotherapeutic agent or class of agents. In certain embodiments, expression levels of one or more MEDIATOR complex and/or SWI/SNF complex genes (e.g., MED12, SMARCE1, and/or ARIDA1) are measured in one or more cancer cells of a patient in combination with an array profile, such as a CGH (comparative genomic hybridization) array analysis.

In certain embodiments, the invention provides methods and compositions for identifying a cancer patient who would likely not benefit from a certain chemotherapeutic treatment. For example, an aspect of the invention is a method of screening cancer patients to determine those cancer patients more likely to benefit from a particular chemotherapy, such as RTK inhibitor chemotherapy, comprising obtaining a sample of genetic material from a tumor of the patient; and assaying for the presence of a genotype in the patient that is associated with resistance to the particular chemotherapy, the genotype characterized by an inactivating mutation in one or more MEDIATOR complex and/or SWI/SNF complex genes. In some embodiments, the genotype is further characterized by an inactivating mutation in one or more known markers for chemotherapeutic resistance. In some embodiments, the genetic material is nucleic acid that is characterized by a reduced expression (e.g., reduced mRNA levels) of one or more MEDIATOR complex and/or SWI/SNF complex genes. In further embodiments, reduced mRNA levels are assessed by the evaluating the corresponding cDNA.

In a particular embodiment, the instant invention provides methods and compositions for the identification of a lung cancer patient who would likely not benefit from RTK inhibitor chemotherapy (e.g., the patient will be recurrence-free for a period of time less than a patient undergoing the same chemotherapy). In some embodiments, the methods of the instant invention predict whether a chemotherapeutic agent or other compound is likely to be cytotoxic to one or more cancer cells.

Cancers for which the prognostic methods and compositions of the instant invention may provide predictive results for resistance to anticancer treatment include cancers such as breast cancer (e.g., BRCA-1 deficient, stage-III HER2-negative), ovarian cancer (e.g., BRCA-1 deficient, epithelial ovarian cancer), lung cancer (e.g., non-small-cell lung cancer or small cell lung cancer, metastatic non-small cell lung cancer), liver cancer (e.g., hepatocellular carcinoma), head and neck cancer (e.g., metastatic squamous cell carcinoma of the head and neck (SCCHN), squamous cell carcinoma, laryngeal cancer, hypopharyngeal cancer, oropharyngeal cancer, and oral cavity cancer), bladder cancer (e.g., transitional cell carcinoma of the bladder), and colorectal cancer (e.g., advanced (non-resectable locally advanced or metastatic) colorectal cancer). Other cancers for which the methods and compositions of the invention may provide predictive results for resistance to anticancer treatment include cervical cancer (e.g., recurrent and stage IVB), mesothelioma, solid tumors (e.g., advanced solid tumors), renal cell carcinoma (e.g., advanced renal cell carcinoma), stomach cancer, sarcoma, prostate cancer (e.g., hormone refractory prostate cancer), melanoma, thyroid cancer (e.g., papillary thyroid cancer), brain cancer, adenocarcinoma, subependymal giant cell astrocytoma, endometrial cancer, glioma, glioblastoma, and other tumors that have metastasized to the brain, esophageal cancer, neuroblastoma, hematological cancers, and lymphoma.

In some embodiments, the cancer is one in which one or more RTK inhibitor drugs are employed either alone or in combination with other chemotherapeutic agents as a part of an anticancer treatment regimen. In other embodiments, the cancer is one in which one or more RTK inhibitor drugs are employed either alone or in combination with additional treatment regimens, such as surgical procedures, radiation, and/or other anticancer treatments. In certain embodiments, the cancer is one in which one or more RTK inhibitor agents are used as a first-line form of treatment. In yet other embodiments, the one or more RTK inhibitor drugs are employed in combination with an inhibitor of the TGF-beta pathway.

In certain embodiments, the instant invention relates to methods and compositions encompassing the detection of expression levels of a MEDIATOR complex and/or SWI/SNF complex and/or RAS-GAP gene in one or more cells of a subject. Typically, the subject is a human patient who has or is suspected of having at least one type of cancer, and the expression levels of the MEDIATOR complex and/or SWI/SNF complex and/or RAS-GAP gene are detected in a sample of one or more cells, typically one or more tumor cells, from the human patient, which are then compared with the expression levels of the MEDIATOR complex and/or SWI/SNF complex and/or RAS-GAP gene in a control sample. A control sample will generally be one in which the MEDIATOR complex and/or SWI/SNF complex and/or RAS-GAP gene expression levels are known and correlated with resistance to anticancer treatment to a certain drug or group of drugs. In some embodiments, the control sample is one in which the MEDIATOR complex and/or SWI/SNF complex and/or RAS-GAP gene expression levels are known and correlated with a lack of resistance to anticancer treatment to a certain drug or group of drugs. In certain embodiments, the MEDIATOR complex and/or SWI/SNF complex and/or RAS-GAP gene expression levels in one or more tumor cells of a patient are compared with the expression levels in one or more normal cells of the patient, wherein a reduced expression in the one or more tumor cells in comparison to the normal cells of the patient are predictive of resistance to anticancer treatment to a certain drug or group of drugs. In some embodiments, more than one control sample is used for comparative purposes with the test sample from the subject. In certain embodiments, the expression levels of a MEDIATOR complex gene are detected. In other embodiments, the expression levels of a SWI/SNF complex gene are detected. In yet other embodiments, the expression levels of a RAS-GAP gene are detected.

In certain embodiments, the invention relates to a method for predicting a lung cancer patient's response to RTK inhibitor drug chemotherapy, such as gefitinib or erlotinib treatment. In some embodiments, the lung cancer patient has not yet received RTK inhibitor drug chemotherapy. In further embodiments, a sample of the lung cancer cells from the patient is analyzed for the levels of expression of a MEDIATOR complex and/or SWI/SNF complex gene, such as MED12, SMARCE1, and/or ARIDA1, and or a RAS-GAP gene, such as DAB2IP, NF1, and/or RASAL3. If expression levels of the MEDIATOR complex and/or SWI/SNF complex gene (e.g., MED12, SMARCE1, and/or ARIDA1) and/or RAS-GAP gene (e.g., DAB2IP, NF1, and/or RASAL3) are low compared to expression levels in normal lung tissue, then the lung cancer cells in the patient are likely resistant to RTK inhibitor anticancer treatment.

In certain embodiments, the expression level of the MEDIATOR complex and/or SWI/SNF complex gene, such as MED12, SMARCE1, and/or ARIDA1, and/or RAS-GAP gene, such as DAB2IP, NF1, and/or RASAL3 in cancer tissue is lower than the expression level of the gene in normal tissue. In predicting resistance to anticancer treatment of a tumor, cut-off levels of expression may be determined empirically for the subject cancer for which resistance to anticancer treatment is being assessed.

In other embodiments, the instant invention relates to methods and compositions encompassing the detection of one or more inactivating mutations in a MEDIATOR complex and/or SWI/SNF complex and/or RAS-GAP gene in one or more cells of a subject. Typically, the subject is a human patient who has or is suspected of having at least one type of cancer, and the nucleic acid of the MEDIATOR complex and/or SWI/SNF complex and/or RAS-GAP are isolated from a sample of one or more cells, typically one or more tumor cells, from the human patient, which are then compared with the nucleic acid of the MEDIATOR complex and/or SWI/SNF complex and/or RAS-GAP in a control sample. A control sample will generally be one in which the MEDIATOR complex and/or SWI/SNF complex and/or RAS-GAP nucleic acid sequences are known and correlated with resistance to anticancer treatment to a certain drug or group of drugs. In some embodiments, the control sample is one in which the MEDIATOR complex and/or SWI/SNF complex and/or RAS-GAP nucleic acid sequences are known and correlated with a lack of resistance to anticancer treatment to a certain drug or group of drugs. In some embodiments, more than one control sample is used for comparative purposes with the test sample from the subject. In certain embodiments, the inactivating mutation is a point mutation. In some embodiments, the inactivating mutation is a hypomorphic mutation. In other embodiments, the inactivating mutation is a gene deletion. In yet other embodiments, the inactivating mutation is an amplification.

In some embodiments, the instant invention relates to methods and compositions encompassing evaluating the protein activity and/or sequence and/or posttranslational modification state of one or more RAS-GAP proteins and/or proteins in a MEDIATOR complex and/or SWI/SNF complex in one or more cells of a subject. Typically, the subject is a human patient who has or is suspected of having at least one type of cancer, and the RAS-GAP protein and/or protein of the MEDIATOR complex and/or SWI/SNF complex is isolated from a sample of one or more cells, typically one or more tumor cells, from the human patient, which are then compared with the RAS-GAP protein and/or protein of the MEDIATOR complex and/or SWI/SNF complex in a control sample. A control sample will generally be one in which the RAS-GAP protein and/or MEDIATOR complex and/or SWI/SNF complex protein sequences and/or activity and/or posttranslational modification state are known and correlated with resistance to anticancer treatment to a certain drug or group of drugs. In some embodiments, the control sample is one in which the RAS-GAP protein and/or MEDIATOR complex and/or SWI/SNF complex protein sequences and/or activity and/or posttranslational modification state are known and correlated with a lack of resistance to anticancer treatment to a certain drug or group of drugs.

Evaluation of protein activity includes assaying the enzymatic activity of the protein. In certain embodiments, the posttranslational modification status of the protein is assessed. In further embodiments, one or more posttranslational modifications (or lack thereof) is associated with protein dysfunction, such as reduced enzymatic activity by the protein. In some embodiments, the RAS-GAP and/or MEDIATOR complex and/or SWI/SNF complex protein in one or more cells of a subject is dysfunctional, and this dysfunction is indicative of resistance to one or more anticancer treatments. Examples of protein dysfunction include reduced or no enzymatic and/or binding activity of the protein; reduced or no protein expression; and/or improper protein modification, such as phosphorylation that results in inactivity of the protein.

The terms “marker” and “biomarker” are used interchangeably herein and refer to a gene, protein, or fragment thereof, the expression or level or activity of which changes between certain conditions. Where the expression or level or activity of the gene, protein, or fragment thereof correlates with a certain condition, the gene, protein, or fragment thereof is a marker for that condition.

“Resistant,” “resistance,” or “resistance to anticancer treatment” in the context of treatment of a cancer cell with a chemotherapeutic agent or other compound means that the chemotherapeutic agent or other compound is not likely to have an optimal effect on the cancer cell. In some embodiments, the compound is not likely to have any effect on the cancer cells. In certain embodiments, the effect of a compound on one or more cancer cells is reduced. In certain further embodiments, a tumor is likely to be less sensitive to a compound but not completely resistant to it. In certain embodiments, the compound is not likely to be cytotoxic to the cancer cell. In some embodiments, the compound is not cytotoxic to the cancer cell.

By “primary resistance” with regard to one or more cancer cells in a patient is meant cells that are naïve for anticancer treatment. For example, a tumor that demonstrates primary resistance to an anticancer treatment includes one that has never been treated with the anticancer drug or drugs but demonstrates or is predicted to demonstrate resistance to the anticancer drug or drugs once treatment has begun.

By “secondary resistance” with regard to one or more cancer cells in a patient is meant cells that have acquired resistance to an anticancer treatment. For example, a tumor that demonstrates secondary resistance to an anticancer treatment includes one that has been treated for a prolonged period of time with one or more anticancer drugs but resistance arises to the one or more anticancer drugs after treatment.

By “inactivating mutation” is meant a mutation in, for example, a nucleic acid that encodes a protein that is inactive. This includes, for example, mutations that result in the loss of protein expression and/or activity and includes genetic mutations such as point mutations, translocations, amplifications, deletions (including whole gene deletions), and hypomorphic mutations (e.g., where an altered gene product possesses a reduced level of activity or where the wild-type gene product is expressed at a reduced level). “Inactivating mutation” also includes biomarker dysfunctions due to post-translational protein regulation, for example, where a protein biomarker is inactive or exhibits impaired activity due to, for example, one or more posttranslational modifications, such as phosphorylation that results in protein inactivity.

The term “biomarker dysfunction” with regard to a protein or protein fragment refers to dysfunction of the protein or fragment thereof as a result of improper regulation at the posttranslational level, such as, for example, phosphorylation that results in protein inactivity.

By “MEDIATOR complex gene” is meant any gene encoding for a protein of the MEDIATOR complex.

By “reference MEDIATOR complex gene” is meant a MEDIATOR complex gene in a control sample, e.g., a normal sample such as a non-cancerous tissue sample. Typically, the expression levels of a reference MEDIATOR complex gene serve as a reference for comparative purposes with the levels of expression of the same MEDIATOR complex gene in a different sample, typically a test sample, such as a lung tumor sample.

By “SWI/SNF complex gene” is meant any gene encoding for a protein of the SWI/SNF complex.

By “reference SWI/SNF complex gene” is meant a SWI/SNF complex gene in a control sample, e.g., a normal sample such as a non-cancerous tissue sample. Typically, the expression levels of a reference SWI/SNF complex gene serve as a reference for comparative purposes with the levels of expression of the same SWI/SNF complex gene in a different sample, typically a test sample, such as a lung tumor sample.

By “RAS-GAP gene” is meant any gene encoding for a RAS-GAP protein.

By “reference RAS-GAP gene” is meant a RAS-GAP gene in a control sample, e.g., a normal sample such as a non-cancerous tissue sample. Typically, the expression levels of a reference RAS-GAP gene serve as a reference for comparative purposes with the levels of expression of the same RAS-GAP gene in a different sample, typically a test sample, such as a lung tumor sample.

By “TGFβ pathway gene” is meant any gene encoding for a protein in the TGFβ signaling pathway.

By “TGFβ pathway target gene” is meant any gene whose expression is regulated by TGFβ signaling.

By “reference TGFβ pathway gene” is meant a TGFβ signaling pathway gene in a control sample, e.g., a normal sample such as a non-cancerous tissue sample. Typically, the expression levels of a reference TGFβ pathway gene serve as a reference for comparative purposes with the levels of expression of the same TGFβ pathway gene in a different sample, typically a test sample, such as a lung tumor sample.

By “MED12^{KD} signature” is meant the nucleic acid expression profile depicted in FIG. 37. FIG. 37 depicts the genes deregulated by MED12^{KD} (>2 fold) in at least three out of five cell lines used. The term “MED12^{KD} signature” includes the 237 upregulated genes and 22 downregulated genes depicted in FIG. 37, as well as any protein products of these genes.

By “positive reference MED12KD signature nucleic acid and/or proteins” is meant the nucleic acid expression profile of one or more genes depicted in FIG. 37 in one or more independent control sample cells known to be resistant to an anticancer treatment, e.g., one or more cells of a cancer cell line or a tumor sample. Typically, the expression levels of a positive reference MED12KD signature gene serve as a reference for comparative purposes with the levels of expression of the same MED12KD signature gene in a different sample, typically a test sample, such as a lung tumor sample.

By “negative reference MED12KD signature nucleic acid and/or proteins” is meant the nucleic acid expression profile of one or more genes depicted in FIG. 37 in one or more independent control sample cells know to be sensitive to an anticancer treatment, e.g., a normal sample such as a non-cancerous tissue sample. Typically, the expression levels of a negative reference MED12KD signature gene serve as a reference for comparative purposes with the levels of expression of the same MED12KD signature gene in a different sample, typically a test sample, such as a lung tumor sample. In some embodiments, the control sample cell is derived from a tumor sample from a patient prior to chemotherapeutic treatment. The control sample in these embodiments can serve as a reference for comparative purposes with the levels of expression of the same MED12KD signature gene in a different sample cell that is derived from a tumor sample from the patient after chemotherapeutic treatment. In other embodiments, the control sample is the average expression of the FIG. 37 genes that is determined in a collection of tumor or cell line samples. The term “negative reference MED12KD signature” likewise includes the expression levels of a random set of genes in the test sample. In these embodiments, the random set of genes from the test sample, which may include one or more of the genes depicted in FIG. 37, are used for comparative purposes with the expression levels of the genes depicted in FIG. 37 in the test sample.

The term “EMT-like phenotype” refers to a partial epithelial-mesenchymal transition (EMT), leading to the induction of mesenchymal markers such as vimentin (VIM) and N-cadherin (CDH2), but not the loss of at least one epithelial marker, such as E-cadherin. As described herein, MED12^{KD} causes expression of the mesenchymal markers VIM and CDH2, indicating that an EMT-like process is initiated in MED12^{KD} cells.

By “interact directly” is meant that a protein or other molecular compound binds and/or enzymatically interacts with a target protein. For example, MEK1 interacts directly with ERK.

By “interact indirectly” is meant that a protein or other molecular compound binds and/or enzymatically interacts with a cellular protein or other molecular compound that may itself interact with a second cellular protein and so forth until a final cellular protein interacts directly with a target protein. This includes any upstream activators of a target protein, such as ERK, in a signaling cascade, such as a receptor tyrosine kinase signaling cascade. Examples of proteins that interact indirectly with ERK include A-RAF, B-RAF, RAF1, MOS, RTKs, and G-protein-coupled receptors.

By “similar” in the context of the expression of one or more nucleic acid and/or proteins is meant that the expression levels of one or more nucleic acid and/or proteins in one sample is the same as or about the same as the expression levels of the one or more nucleic acid and/or proteins in a second sample. In certain embodiments, the expression levels of a gene are the same (e.g., no measurable difference) between two different samples. In other embodiments, the expression levels of a gene are about the same (e.g., within experimental margins of error) between two different samples.

In various aspects, determination of a level of expression of nucleic acid and/or protein in a test sample that is the same, greater than, or less than that produced by the corresponding nucleic, acid and/or protein in a positive reference MED12KD signature is indicative of resistance to anticancer treatment in the tumor from which the test sample was derived. Accordingly, in certain embodiments detection of signal intensity from a test sample that is the same, within experimentally acceptable margins of error, as the signal intensity produced by the positive reference MED12KD signature sample is sufficient to classify the tumor from which the test sample was produced as anticancer treatment resistant. In certain embodiments, detection of signal intensity from a test sample that is greater, within experimentally acceptable margins of error, than the signal intensity produced by the positive reference MED12KD signature sample is sufficient to classify the tumor from which the test sample was produced as anticancer treatment resistant. In certain embodiments, detection of signal intensity from a test sample that is less, within experimentally acceptable margins of error, than the signal intensity produced by the positive reference MED12KD signature sample is sufficient to classify the tumor from which the test sample was produced as anticancer treatment resistant.

In certain embodiments, the deviation of signal intensity of the test sample from the positive reference MED12KD signature sample is measured as a percent difference. In certain embodiments, a test sample is deemed to have produced a signal that is greater than the positive reference MED12KD signature sample if the signal intensity of the test sample measures at a level selected from: the signal intensity of the positive reference MED12KD signature sample greater than 1%; greater than 2%; greater than 5%; greater than 10%; greater than 15%; greater than 20%; the greater than 25%; greater than 30%; greater than 35%; greater than 40%; greater than 45%; greater than 50%; greater than 55%; greater than 60%; greater than 65%; greater than 70%; greater than 75%; greater than 80%; greater than 85%; greater than 90%; greater than 95%; or greater than 100%.

In certain embodiments, a test sample is deemed to have produced a signal that is less than the positive reference MED12KD signature sample if the signal intensity of the test sample measures at a level selected from: the signal intensity of the reference sample less 1%; less 2%; less 5%; less 10%; less 15%; less 20%; less 25%; less 30%; less 35%; less 40%; less 45%; less 50%; less 55%; less 60%; less 65%; less 70%; less 75%; less 80%; less 85%; less 90%; less 95%; or less 100% (or no signal produced by the test sample).

In certain embodiments, the deviation of signal intensity of the test sample from the positive reference MED12KD signature sample is measured as a-fold difference, or a difference based upon unit signal production. In certain embodiments, a test sample is deemed to have produced a signal that is greater than the positive reference MED12KD signature sample if the signal intensity of the test sample is selected from: two-fold greater than; three-fold greater than; four-fold greater than; five-fold greater than; six-fold greater than; seven-fold greater than; eight-fold greater than; nine-fold greater than; ten-fold greater; and more than ten-fold greater than the signal intensity of the positive reference MED12KD signature sample.

In certain embodiments, a test sample is deemed to have produced a signal that is less than the positive reference MED12KD signature sample if the signal intensity of the test sample is selected from: two-fold less than; three-fold less than; four-fold less than; five-fold less than; six-fold less than; seven-fold less than; eight-fold less than; nine-fold less than; ten-fold less than; and greater than ten-fold less than the signal intensity of the positive reference MED12KD signature sample.

In certain embodiments where the expression of a nucleic acid and/or protein in a test sample is compared with the expression level of the same nucleic acid and/or protein in a positive reference MED12KD signature nucleic acid and/or protein sample, expression of the test sample nucleic acid and/or protein that is the same as (e.g., no measurable difference) or greater than (e.g., more than 10-fold greater than) the expression level of the nucleic acid and/or protein corresponding to an upregulated gene in the positive reference MED12KD signature, then resistance to anticancer treatment in the test sample is indicated.

In certain embodiments where the expression of a nucleic acid and/or protein in a test sample is compared with the expression level of the same nucleic acid and/or protein in a positive reference MED12KD signature nucleic acid and/or protein, expression of the test sample nucleic acid and/or protein that is the same as (e.g., no measurable difference) or less than (e.g., more than 10-fold less than) the expression level of the nucleic acid and/or protein corresponding to a downregulated gene in the positive reference MED12KD signature, then resistance to anticancer treatment in the test sample is indicated.

In various aspects, determination of a level of expression of nucleic acid and/or protein in a test sample that is greater than or less than that produced by the corresponding nucleic acid and/or protein in a negative reference MED12KD signature is indicative of resistance to anticancer treatment in the tumor from which the test sample was derived. Accordingly, in certain embodiments, detection of signal intensity from a test sample that is greater, within experimentally acceptable margins of error, than the signal intensity produced by the negative reference MED12KD signature sample is sufficient to classify the tumor from which the test sample was produced as anticancer treatment resistant. In certain embodiments, detection of signal intensity from a test sample that is less, within experimentally acceptable margins of error, than the signal intensity produced by the negative reference MED12KD signature sample is sufficient to classify the tumor from which the test sample was produced as anticancer treatment resistant.

In certain embodiments, the deviation of signal intensity of the test sample from the negative reference MED12KD signature sample is measured as a percent difference. In certain embodiments, a test sample is deemed to have produced a signal that is greater than the positive reference MED12KD signature sample if the signal intensity of the test sample measures at a level selected from: the signal intensity of the positive reference MED12KD signature sample greater than 1%, greater than 2%, greater than 5%; greater than 10%; greater than 15%; greater than 20%; the greater than 25%; greater than 30%; greater than 35%; greater than 40%; greater than 45%; greater than 50%; greater than 55%; greater than 60%; greater than 65%; greater than 70%; greater than 75%; greater than 80%; greater than 85%; greater than 90%; greater than 95%; or greater than 100%.

In certain embodiments, a test sample is deemed to have produced a signal that is less than the negative reference MED12KD signature sample if the signal intensity of the test sample measures at a level selected from: the signal intensity of the reference sample less 1%, less 2%, less 5%; less 10%; less 15%; less 20%; less 25%; less 30%; less 35%; less 40%; less 45%; less 50%; less 55%; less 60%; less 65%; less 70%; less 75%; less 80%; less 85%; less 90%; less 95%; or less 100% (or no signal produced by the test sample).

In certain embodiments, the deviation of signal intensity of the test sample from the negative reference MED12KD signature sample is measured as a-fold difference, or a difference based upon unit signal production. In certain embodiments, a test sample is deemed to have produced a signal that is greater than the negative reference MED12KD signature sample if the signal intensity of the test sample is selected from: one-fold greater than; one-and-half-fold greater than; two-fold greater than; three-fold greater than; four-fold greater than; five-fold greater than; six-fold greater than; seven-fold greater than; eight-fold greater than; nine-fold greater than; ten-fold greater; and more than ten-fold greater than the signal intensity of the negative reference MED12KD signature sample.

In certain embodiments, a test sample is deemed to have produced a signal that is less than the negative reference MED12KD signature sample if the signal intensity of the test sample is selected from: one-fold less than; one-and-half-fold less than; two-fold less than; three-fold less than; four-fold less than; five-fold less than; six-fold less than; seven-fold less than; eight-fold less than; nine-fold less than; ten-fold less than; and greater than ten-fold less than the signal intensity of the negative reference MED12KD signature sample.

In certain embodiments where the expression of a nucleic acid and/or protein in a test sample is compared with the expression level of the same nucleic acid and/or protein in a negative reference MED12KD signature nucleic acid and/or protein sample, expression of the test sample nucleic acid and/or protein that is greater than (e.g., more than 1.2-fold greater than) the expression level of the nucleic acid and/or protein corresponding to an upregulated gene in the negative reference MED12KD signature, then resistance to anticancer treatment in the test sample is indicated.

In certain embodiments where the expression of a nucleic acid and/or protein in a test sample is compared with the expression level of the same nucleic acid and/or protein in a negative reference MED12KD signature nucleic acid and/or protein, expression of the test sample nucleic acid and/or protein that is less than (e.g., more than 1.2-fold less than) the expression level of the nucleic acid and/or protein corresponding to a downregulated gene in the negative reference MED12KD signature, then resistance to anticancer treatment in the test sample is indicated.

As used herein, the terms “drug,” “agent,” and “compound,” either alone or together with “chemotherapeutic” or “chemotherapy,” encompass any composition of matter or mixture which provides some pharmacologic effect that can be demonstrated in-vivo or in vitro. This includes small molecules, antibodies, microbiologicals, vaccines, vitamins, and other beneficial agents. As used herein, the terms further include any physiologically or pharmacologically active substance that produces a localized or systemic effect in a patient.

The term “nucleic acid” encompasses DNA, RNA (e.g., mRNA, tRNA), heteroduplexes, and synthetic molecules capable of encoding a polypeptide and includes all analogs and backbone substitutes such as PNA that one of ordinary skill in the art would recognize as capable of substituting for naturally occurring nucleotides and backbones thereof. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences which encode a particular amino acid sequence.

Unless otherwise indicated, nucleic acids are written left to right in 5′ to 3′ orientation; amino acid sequences are written left to right in amino to carboxy orientation, respectively.

“Antisense” nucleic acids are DNA or RNA molecules that are complementary to at least a portion of a specific mRNA molecule (Weintraub, Scientific American 262 40, 1990). In the cell, the antisense nucleic acids hybridize to the corresponding mRNA, forming a double-stranded molecule. This interferes with the translation of the mRNA since the cell will not translate an mRNA that is double-stranded. Antisense oligomers of at least about 15, about 20, about 25, about 30, about 35, about 40, or of at least about 50 nucleotides are preferred, since they are easily synthesized and are less likely to cause non-specific interference with translation than larger molecules. The use of antisense methods to inhibit the in vitro translation of genes is well known in the art (Marcus-Sakura Anal. Biochem. 172: 289, 1998).

Short double-stranded RNAs (dsRNAs; typically <30 nucleotides) can be used to silence the expression of target genes in animals and animal cells. Upon introduction, the long dsRNAs enter the RNA interference (RNAi) pathway which involves the production of shorter (20-25 nucleotide) small interfering RNAs (siRNAs) and assembly of the siRNAs into RNA-induced silencing complexes (RISCs). The siRNA strands are then unwound to form activated RISCs, which cleave the target RNA. Double stranded RNA has been shown to be extremely effective in silencing a target RNA.

General methods of using antisense, ribozyme technology and RNAi technology, to control gene expression, or of gene therapy methods for expression of an exogenous gene in this manner are well known in the art. Each of these methods utilizes a system, such as a vector, encoding either an antisense or ribozyme transcript. The term “RNAi” stands for RNA interference. This term is understood in the art to encompass technology using RNA molecules that can silence genes. See, for example, McManus, et al. Nature Reviews Genetics 3: 737, 2002. In this application, the term “RNAi” encompasses molecules such as small interfering or short interfering RNA (siRNA), small hairpin or short hairpin RNA (shRNA), microRNAs, and small temporal RNA (stRNA). Generally speaking, RNA interference results from the interaction of double-stranded RNA with genes.

The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. In certain embodiments, siRNA molecules are 12-28 nucleotides long, more preferably 15-25 nucleotides long, still more preferably 19-23 nucleotides long and most preferably 21-23 nucleotides long. In certain embodiments, preferred siRNA molecules are 12, 13, 14, 15, 16, 17, 18, 19, 20, 21, 22, 23, 24, 25, 26, 27 28 or 29 nucleotides in length.

As used herein, the term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter code for amino acid residues are used herein.

As used herein, a “synthetic” molecule is produced by in vitro chemical or enzymatic synthesis rather than by an organism.

As used herein, the term “expression” refers to the process by which a polypeptide is produced based on the nucleic acid sequence of a gene. The process includes both transcription and translation. The term “expression” also includes the protein product of a translated mRNA. The term “expression” as it refers to protein includes both protein levels and protein activity (e.g., protein binding, enzymatic activity, etc.). The term “expression” also refers to the transcription of non-translated nucleic acid (e.g., non-coding mRNA).

A “gene” refers to the DNA segment encoding a polypeptide or RNA.

By “homolog” is meant an entity having a certain degree of identity with the subject amino acid sequences and the subject nucleotide sequences. As used herein, the term “homolog” covers identity with respect to structure and/or function, for example, the expression product of the resultant nucleotide sequence has the enzymatic activity of a subject amino acid sequence. With respect to sequence identity, preferably there is at least 70%, 75%, 80%, 81%, 82%, 83%, 84%, 85%, 86%, 87%, 88%, 89%, 90%, 91%, 92%, 93%, 94%, 95%, 96%, 97%, 98%, or even 99% sequence identity. These terms also encompass allelic variations of the sequences. The term, homolog, may apply to the relationship between genes separated by the event of speciation or to the relationship between genes separated by the event of genetic duplication.

Relative sequence identity can be determined by commercially available computer programs that can calculate % identity between two or more sequences using any suitable algorithm for determining identity, using, for example, default parameters. A typical example of such a computer program is CLUSTAL. Advantageously, the BLAST algorithm is employed, with parameters set to default-values. The BLAST algorithm is described in detail on the National Center for Biotechnology Information (NCBI) website.

The homologs of the peptides as provided herein typically have structural similarity with such peptides. A homolog of a polypeptide includes one or more conservative amino acid substitutions, which may be selected from the same or different members of the class to which the amino acid belongs.

In one embodiment, the sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

The present invention also encompasses conservative substitution (substitution and replacement are both used herein to mean the interchange of an existing amino acid residue with an alternative residue) that may occur e.g., like-for-like substitution such as basic for basic, acidic for acidic, polar for polar, etc. Non-conservative substitution may also occur e.g., from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine. Conservative substitutions that may be made are, for example, within the groups of basic amino acids (Arginine, Lysine and Histidine), acidic amino acids (glutamic acid and aspartic acid), aliphatic amino acids (Alanine, Valine, Leucine, Isoleucine), polar amino acids (Glutamine, Asparagine, Serine, Threonine), aromatic amino acids (Phenylalanine, Tryptophan and Tyrosine), hydroxylamino acids (Serine, Threonine), large amino acids (Phenylalanine and Tryptophan) and small amino acids (Glycine, Alanine).

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### Methods of Detecting Expression Levels

There are many methods known in the art for determining the genotype of a patient. Any method for determining genotype can be used for determining genotypes in the present invention. Such methods include, but are not limited to, amplimer sequencing, DNA sequencing, fluorescence spectroscopy, fluorescence resonance energy transfer (or “FRET”)-based hybridization analysis, high throughput screening, mass spectroscopy, nucleic acid hybridization, polymerase chain reaction (PCR), RFLP analysis and size chromatography (e.g., capillary or gel chromatography), all of which are well known to one of ordinary skill in the art.

Many methods of sequencing genomic DNA are known in the art, and any such method can be used, see for example Sambrook et al., Molecular Cloning; A Laboratory Manual 2d ed. (1989). For example, a DNA fragment of interest can be amplified using the polymerase chain reaction or some other cyclic polymerase mediated amplification reaction. The amplified region of DNA can then be sequenced using any method known in the art. Advantageously, the nucleic acid sequencing is by automated methods (reviewed by Meldrum, Genome Res. September 2000; 10(9):1288-303, the disclosure of which is incorporated by reference in its entirety), for example using a Beckman CEQ 8000 Genetic Analysis System (Beckman Coulter Instruments, Inc.). Methods for sequencing nucleic acids include, but are not limited to, automated fluorescent DNA sequencing (see, e.g., Watts & MacBeath, Methods Mol. Biol. 2001; 167:153-70 and MacBeath et al., Methods Mol. Biol. 2001; 167:119-52), capillary electrophoresis (see, e.g., Bosserhoff et al., Comb Chem High Throughput Screen. December 2000; 3(6):455-66), DNA sequencing chips (see, e.g., Jain, Pharmacogenomics. August 2000; 1(3):289-307), mass spectrometry (see, e.g., Yates, Trends Genet. January 2000; 16(1):5-8), pyrosequencing (see, e.g., Ronaghi, Genome Res. January 2001; 11(1):3-11), and ultrathin-layer gel electrophoresis (see, e.g., Guttman & Ronai, Electrophoresis. December 2000; 21 (18):3952-64), the disclosures of which are hereby incorporated by reference in their entireties. The sequencing can also be done by any commercial company. Examples of such companies include, but are not limited to, the University of Georgia Molecular Genetics Instrumentation Facility (Athens, Ga.) or SeqWright DNA Technologies Services (Houston, Tex.).

Any one of the methods known in the art for amplification of DNA may be used, such as for example, the polymerase chain reaction (PCR), the ligase chain reaction (LCR) (Barany, F., Proc. Natl. Acad. Sci. (U.S.A.) 88:189-193 (1991)), the strand displacement assay (SDA), or the oligonucleotide ligation assay (“OLA”) (Landegren, U. et al., Science 241:1077-1080 (1988)). Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990)). Other known nucleic acid amplification procedures, such as transcription-based amplification systems (Malek, L. T. et al., U.S. Pat. No. 5,130,238; Davey, C. et al., European Patent Application 329,822; Schuster et al., U.S. Pat. No. 5,169,766; Miller, H. I. et al., PCT Application WO89/06700; Kwoh, D. et al., Proc. Natl. Acad. Sci. (U.S.A.) 86:1173 (1989); Gingeras, T. R. et al., PCT Application WO88/10315)), or isothermal amplification methods (Walker, G. T. et al., Proc. Natl. Acad. Sci. (U.S.A.) 89:392-396 (1992)) may also be used.

To perform a cyclic polymerase mediated amplification reaction according to the present invention, the primers are hybridized or annealed to opposite strands of the target DNA, the temperature is then raised to permit the thermostable DNA polymerase to extend the primers and thus replicate the specific segment of DNA spanning the region between the two primers. Then the reaction is thermocycled so that at each cycle the amount of DNA representing the sequences between the two primers is doubled, and specific amplification of gene DNA sequences, if present, results.

Any of a variety of polymerases can be used in the present invention. For thermocyclic reactions, the polymerases are thermostable polymerases such as Taq, KlenTaq, Stoffel Fragment, Deep Vent, Tth, Pfu, Vent, and UlTma, each of which are readily available from commercial sources. For non-thermocyclic reactions, and in certain thermocyclic reactions, the polymerase will often be one of many polymerases commonly used in the field, and commercially available, such as DNA pol 1, Klenow fragment, T7 DNA polymerase, and T4 DNA polymerase. Guidance for the use of such polymerases can readily be found in product literature and in general molecular biology guides.

Typically, the annealing of the primers to the target DNA sequence is carried out for about 2 minutes at about 37-55° C., extension of the primer sequence by the polymerase enzyme (such as Taq polymerase) in the presence of nucleoside triphosphates is carried out for about 3 minutes at about 70-75° C., and the denaturing step to release the extended primer is carried out for about 1 minute at about 90-95° C. However, these parameters can be varied, and one of skill in the art would readily know how to adjust the temperature and time parameters of the reaction to achieve the desired results. For example, cycles may be as short as 10, 8, 6, 5, 4.5, 4, 2, 1, 0.5 minutes or less.

Also, “two temperature” techniques can be used where the annealing and extension steps may both be carried out at the same temperature, typically between about 60-65° C., thus reducing the length of each amplification cycle and resulting in a shorter assay time.

Typically, the reactions described herein are repeated until a detectable amount of product is generated. Often, such detectable amounts of product are between about 10 ng and about 100 ng, although larger quantities, e.g. 200 ng, 500 ng, 1 mg or more can also, of course, be detected. In terms of concentration, the amount of detectable product can be from about 0.01 pmol, 0.1 pmol, 1 pmol, 10 pmol, or more. Thus, the number of cycles of the reaction that are performed can be varied, the more cycles are performed, the more amplified product is produced. In certain embodiments, the reaction comprises 2, 5, 10, 15, 20, 30, 40, 50, or more cycles.

For example, the PCR reaction may be carried out using about 25-50 μl samples containing about 0.01 to 1.0 ng of template amplification sequence, about 10 to 100 pmol of each generic primer, about 1.5 units of Taq DNA polymerase (Promega Corp.), about 0.2 mM dDATP, about 0.2 mM dCTP, about 0.2 mM dGTP, about 0.2 mM dTTP, about 15 mM MgCl.sub.2, about 10 mM Tris-HCl (pH 9.0), about 50 mM KCl, about 1 μg/ml gelatin, and about 10 μl/ml Triton X-100 (Saiki, 1988).

Those of ordinary skill in the art are aware of the variety of nucleotides available for use in the cyclic polymerase mediated reactions. Typically, the nucleotides will consist at least in part of deoxynucleotide triphosphates (dNTPs), which are readily commercially available. Parameters for optimal use of dNTPs are also known to those of skill, and are described in the literature. In addition, a large number of nucleotide derivatives are known to those of skill and can be used in the present reaction. Such derivatives include fluorescently labeled nucleotides, allowing the detection of the product including such labeled nucleotides, as described below. Also included in this group are nucleotides that allow the sequencing of nucleic acids including such nucleotides, such as chain-terminating nucleotides, dideoxynucleotides and boronated nuclease-resistant nucleotides. Commercial kits containing the reagents most typically used for these methods of DNA sequencing are available and widely used. Other nucleotide analogs include nucleotides with bromo-, iodo-, or other modifying groups, which affect numerous properties of resulting nucleic acids including their antigenicity, their replicatability, their melting temperatures, their binding properties, etc. In addition, certain nucleotides include reactive side groups, such as sulfhydryl groups, amino groups, N-hydroxysuccinimidyl groups, that allow the further modification of nucleic acids comprising them.

In certain embodiments, oligonucleotides that can be used as primers to amplify specific nucleic acid sequences of a gene in cyclic polymerase-mediated amplification reactions, such as PCR reactions, consist of oligonucleotide fragments. Such fragments should be of sufficient length to enable specific annealing or hybridization to the nucleic acid sample. The sequences typically will be about 8 to about 44 nucleotides in length, but may be longer. Longer sequences, e.g., from about 14 to about 50, are advantageous for certain embodiments.

In embodiments where it is desired to amplify a fragment of DNA, primers having contiguous stretches of about 8, 9, 10, 11, 12, 13, 14, 15, 16, 17, 18, 19, 20, 21, 22, 23, or 24 nucleotides from a gene sequence are contemplated.

As used herein, “hybridization” refers to the process by which one strand of nucleic acid base pairs with a complementary strand, as occurs during blot hybridization techniques and PCR techniques.

Whichever probe sequences and hybridization methods are used, one ordinarily skilled in the art can readily determine suitable hybridization conditions, such as temperature and chemical conditions. Such hybridization methods are well known in the art. For example, for applications requiring high selectivity, one will typically desire to employ relatively stringent conditions for the hybridization reactions, e.g., one will select relatively low salt and/or high temperature conditions, such as provided by about 0.02 M to about 0.10 M NaCl at temperatures of about 50° C. to about 70° C. Such high stringency conditions tolerate little, if any, mismatch between the probe and the template or target strand. It is generally appreciated that conditions can be rendered more stringent by the addition of increasing amounts of formamide. Other variations in hybridization reaction conditions are well known in the art (see for example, Sambrook et al., Molecular Cloning; A Laboratory Manual 2d ed. (1989)).

Hybridization conditions are based on the melting temperature (Tm) of the nucleic acid binding complex, as taught, e.g., in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of ordinary skill in the art, a maximum stringency hybridization can be used to identify or detect identical nucleotide sequences while an intermediate (or low) stringency hybridization can be used to identify or detect similar or related polynucleotide sequences.

In one aspect, the present invention employs nucleotide sequences that can hybridize to another nucleotide sequence under stringent conditions (e.g., 65° C. and 0.1×SSC {1×SSC=0.15 M NaCl, 0.015 M Na3 Citrate pH 7.0). Where the nucleotide sequence is double-stranded, both strands of the duplex, either individually or in combination, may be employed by the present invention. Where the nucleotide sequence is single-stranded, it is to be understood that the complementary sequence of that nucleotide sequence is also included within the scope of the present invention.

Stringency of hybridization refers to conditions under which polynucleic acid hybrids are stable. Such conditions are evident to those of ordinary skill in the field. As known to those of ordinary skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridization reaction is performed under conditions of higher stringency, followed by washes of varying stringency.

As used herein, high stringency includes conditions that permit hybridization of only those nucleic acid sequences that form stable hybrids in 1 M Na+ at 65-68° C. High stringency conditions can be provided, for example, by hybridization in an aqueous solution containing 6×SSC, 5×Denhardt's, 1% SDS (sodium dodecyl sulphate), 0.1 Na+ pyrophosphate and 0.1 mg/ml denatured salmon sperm DNA as non-specific competitor. Following hybridization, high stringency washing may be done in several steps, with a final wash (about 30 minutes) at the hybridization temperature in 0.2-0.1×SSC, 0.1% SDS.

It is understood that these conditions may be adapted and duplicated using a variety of buffers, e.g., formamide-based buffers, and temperatures. Denhardt's solution and SSC are well known to those of ordinary skill in the art as are other suitable hybridization buffers (see, e.g., Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridization conditions are typically determined empirically, as the length and the GC content of the hybridizing pair also play a role.

Nucleic acid molecules that differ from the sequences of the primers and probes disclosed herein, are intended to be within the scope of the invention. Nucleic acid sequences that are complementary to these sequences, or that are hybridizable to the sequences described herein under conditions of standard or stringent hybridization, and also analogs and derivatives are also intended to be within the scope of the invention. Advantageously, such variations will differ from the sequences described herein by only a small number of nucleotides, for example by 1, 2, or 3 nucleotides.

Nucleic acid molecules corresponding to natural allelic variants, homologues (i.e., nucleic acids derived from other species), or other related sequences (e.g., paralogs) of the sequences described herein can be isolated based on their homology to the nucleic acids disclosed herein, for example by performing standard or stringent hybridization reactions using all or a portion of the known sequences as probes. Such methods for nucleic acid hybridization and cloning are well known in the art.

Similarly, a nucleic acid molecule detected in the methods of the invention may include only a fragment of the specific sequences described. Fragments provided herein are defined as sequences of at least 6 (contiguous) nucleic acids, a length sufficient to allow for specific hybridization of nucleic acid primers or probes, and are at most some portion less than a full-length sequence. Fragments may be derived from any contiguous portion of a nucleic acid sequence of choice. Derivatives and analogs may be full length or other than full length, if the derivative or analog contains a modified nucleic acid or amino acid, as described below.

Derivatives, analogs, homologues, and variants of the nucleic acids of the invention include, but are not limited to, molecules comprising regions that are substantially homologous to the nucleic acids of the invention, in various embodiments, by at least about 70%, 80%, 85%, 90%, 95%, 96%, 97%, 98%, or even 99% identity over a nucleic acid sequence of identical size or when compared to an aligned sequence in which the alignment is done by a computer homology program known in the art.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms can be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Alternatively or additionally, the term “homology” or “identity”, for instance, with respect to a nucleotide or amino acid sequence, can indicate a quantitative measure of homology between two sequences. The percent sequence homology can be calculated as (NC_{ref}−N_{dif})*100/−N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N N_{ref}=8; N N_{dif}=2). “Homology” or “identity” can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur & Lipman, Proc Natl Acad Sci USA 1983; 80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being considered equal to uracil (U) in RNA sequences. Without undue experimentation, the skilled artisan can consult with many other programs or references for determining percent homology.

In embodiments where expression of a particular gene is assessed by determining the expression of the protein product of the gene, any suitable assay for detecting protein levels and/or activity may be employed. For example, suitable protein activity assays include ubiquitination assays, kinase assays, protein-binding assays, DNA-binding and unwinding assays, and any other suitable assay for assessing the activity of the protein product of a translated gene according to the invention.

### Sampling

In order to determine the genotype or expression level of a particular SWI/SNF complex and/or MEDIATOR complex gene of a patient according to the methods of the present invention, it may be necessary to obtain a sample of genomic DNA or RNA from that patient. That sample of genomic DNA or RNA may be obtained from a sample of tissue or cells taken from that patient.

A sample may comprise any clinically relevant tissue sample, such as a tumor biopsy or fine needle aspirate, hair (including roots), skin, buccal swabs, saliva, or a sample of bodily fluid, such as blood, plasma, serum, lymph, ascitic fluid, cystic fluid, urine or nipple exudate. The sample may be taken from a human, or, in a veterinary context, from non-human animals such as ruminants, horses, swine or sheep, or from domestic companion animals such as felines and canines.

The tissue sample may be marked with an identifying number or other indicia that relates the sample to the individual patient from which the sample was taken. The identity of the sample advantageously remains constant throughout the methods of the invention thereby guaranteeing the integrity and continuity of the sample during extraction and analysis. Alternatively, the indicia may be changed in a regular fashion that ensures that the data, and any other associated data, can be related back to the patient from whom the data was obtained. The amount/size of sample required is known to those ordinarily skilled in the art.

Generally, the tissue sample may be placed in a container that is labeled using a numbering system bearing a code corresponding to the patient. Accordingly, the genotype of a particular patient is easily traceable.

In one embodiment of the invention, a sampling device and/or container may be supplied to the physician. The sampling device advantageously takes a consistent and reproducible sample from individual patients while simultaneously avoiding any cross-contamination of tissue. Accordingly, the size and volume of sample tissues derived from individual patients would be consistent.

According to the present invention, a sample of genomic DNA or RNA is obtained from the tissue sample of the patient of interest. Whatever source of cells or tissue is used, a sufficient amount of cells must be obtained to provide a sufficient amount of DNA or RNA for analysis. This amount will be known or readily determinable by those ordinarily skilled in the art.

DNA or RNA is isolated from the tissue/cells by techniques known to those ordinarily skilled in the art (see, e.g., U.S. Pat. Nos. 6,548,256 and 5,989,431, Hirota et al., Jinrui Idengaku Zasshi. September 1989; 34(3):217-23 and John et al., Nucleic Acids Res. Jan. 25. 1991; 19(2):408; the disclosures of which are incorporated by reference in their entireties). For example, high molecular weight DNA may be purified from cells or tissue using proteinase K extraction and ethanol precipitation. DNA may be extracted from a patient specimen using any other suitable methods known in the art.

In certain embodiments, target polynucleotide molecules are extracted from a sample taken from an individual afflicted with breast cancer. The sample may be collected in any clinically acceptable manner, but must be collected such that marker-derived polynucleotides (e.g., RNA) are preserved. mRNA or nucleic acids derived therefrom (e.g., cDNA or amplified DNA) are preferably labeled distinguishably from standard or control polynucleotide molecules, and both are simultaneously or independently hybridized to a microarray comprising one or more markers of resistance to anticancer treatment as described above. Alternatively, mRNA or nucleic acids derived therefrom may be labeled with the same label as the standard or control polynucleotide molecules, wherein the intensity of hybridization of each at a particular probe is compared.

Methods for preparing total and poly(A)+ RNA are well known and are described generally in Sambrook et al., MOLECULAR CLONING-A LABORATORY MANUAL (2ND ED.), Vols. 1-3, Cold. Spring Harbor Laboratory, Cold Spring Harbor, N.Y. (1989)) and Ausubel et al., CURRENT PROTOCOLS IN MOLECULAR BIOLOGY, vol. 2, Current Protocols Publishing, New York (1994)).

RNA may be isolated from eukaryotic cells by procedures that involve lysis of the cells and denaturation of the proteins contained therein. Cells of interest include wild-type cells (i.e., non-cancerous), drug-exposed wild-type cells, tumor- or tumor-derived cells, modified cells, normal or tumor cell line cells, and drug-exposed modified cells.

Additional steps may be employed to remove DNA. Cell lysis may be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et al., Biochemistry 18:5294-5299 (1979)). Poly(A)+ RNA is selected by selection with oligo-dT cellulose (see Sambrook et al, MOLECULAR CLONING-A LABORATORY MANUAL (2ND ED.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol.

If desired, RNase inhibitors may be added to the lysis buffer. Likewise, for certain cell types, it may be desirable to add a protein denaturation/digestion step to the protocol.

In certain embodiments, it is desirable to preferentially enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or Sephadex™ (see Ausubel et al., CURRENT PROTOCOLS IN MOLECULAR BIOLOGY, vol. 2, Current Protocols Publishing, New York (1994). Once bound, poly(A)+ mRNA is eluted from the affinity column using 2 mM EDTA/0.1% SDS.

The sample of RNA can comprise a plurality of different mRNA molecules, each different mRNA molecule having a different nucleotide sequence. In a specific embodiment, the RNA sample is a mammalian RNA sample.

In a specific embodiment, total RNA or mRNA from cells are used in the methods of the invention. The source of the RNA can be cells of any animal, human, mammal, primate, non-human animal, dog, cat, mouse, rat, bird, yeast, eukaryote, etc. In specific embodiments, the method of the invention is used with a sample containing total mRNA or total RNA from 1×10⁶ cells or less. In another embodiment, proteins can be isolated from the foregoing sources, by methods known in the art, for use in expression analysis at the protein level.

In certain embodiments, expression of a biomarker according to the invention is measured using multiplex ligation-dependent probe amplification (MLPA) (see, e.g., WO 01/61033 and Schouten, J P et al. (2002) “Relative quantification of 40 nucleic acid sequences by multiplex ligation-dependent probe amplification” Nucleic Acids Res 30, e57) or reverse transcriptase MLPA (RT-MLPA) (see, e.g., Eldering, E et al. (2003) “Expression profiling via novel multiplex assay allows rapid assessment of gene regulation in defined signaling pathways” Nucleic Acids Res 31, e153). In RT-MLPA, mRNA is converted to cDNA by reverse transcriptase, followed by a normal MLPA reaction. In other embodiments, methylation-specific MLPA is employed to detect expression of a biomarker according to the instant invention (see, e.g., Nygren, A O et al. (2005) “Methylation-specific MLPA (MS-MPLA): simultaneous detection of CpG methylation and copy number changes of up to 40 sequences” Nucleic Acids Res 33, 14:e128).

### Arrays

As defined herein, a “nucleic acid array” refers to a plurality of unique nucleic acids (or “nucleic acid members”) attached to a support where each of the nucleic acid members is attached to a support in a unique pre-selected region.

In one embodiment, the nucleic acid member attached to the surface of the support is DNA. In another embodiment, the nucleic acid member attached to the surface of the support is either cDNA or oligonucleotides. In another embodiment, the nucleic acid member attached to the surface of the support is cDNA synthesized by polymerase chain reaction (PCR). In another embodiment, sequences bound to the array can be an isolated oligonucleotide, cDNA, EST or PCR product corresponding to any biomarker of the invention total cellular RNA is applied to the array.

Array technology and the various-techniques and applications associated with it is described generally in numerous textbooks and documents. These include Lemieux et al., 1998, Molecular Breeding 4, 277-289, Schena and Davis. Parallel Analysis with Biological Chips, in PCR Methods Manual (eds. M. Innis, D. Gelfand, J. Sninsky), Schena and Davis, 1999, Genes, Genomes and Chips. In DNA Microarrays: A Practical Approach (ed. M. Schena), Oxford University Press, Oxford, UK, 1999), The Chipping Forecast (Nature Genetics special issue; January 1999 Supplement), Mark Schena (Ed.), Microarray Biochip Technology, (Eaton Publishing Company), Cortes, 2000, The Scientist 14[17]:25, Gwynne and Page, Microarray analysis: the next revolution in molecular biology, Science, 1999 Aug. 6; and Eakins and Chu, 1999, Trends in Biotechnology, 17, 217-218.

Major applications for array technology include the identification of sequence (gene/gene mutation) and the determination of expression level (abundance) of genes. Gene expression profiling may make use of array technology, optionally in combination with proteomics techniques (Celis et al, 2000, FEBS Lett, 480(1):2-16; Lockhart and Winzeler, 2000, Nature 405(6788):827-836; Khan et al., 1999, 20(2):223-9). Other applications of array technology are also known in the art; for example, gene discovery, cancer research (Marx, 2000, Science 289: 1670-1672; Scherf, et-al, 2000, Nat Genet; 24(3):236-44; Ross et al, 2000, Nat. Genet. 2000 March; 24(3):227-35), SNP analysis (Wang et al, 1998, Science, 280(5366):1077-82), drug discovery, pharmacogenomics, disease diagnosis (for example, utilising microfluidics devices: Chemical & Engineering News, Feb. 22, 1999, 77(8):27-36), toxicology (Rockett and Dix (2000), Xenobiotica, 30(2): 155-77; Afshari et al., 1999, Cancer Res1; 59(19):4759-60) and toxicogenomics (a hybrid of functional genomics and molecular toxicology).

In general, any library may be arranged in an orderly manner into an array, by spatially separating the members of the library. Examples of suitable libraries for arraying include nucleic acid libraries (including DNA, cDNA, oligonucleotide, etc. libraries), peptide, polypeptide and protein libraries, as well as libraries comprising any molecules, such as ligand libraries, among others.

The samples (e.g., members of a library) are generally fixed or immobilized onto a solid phase, preferably a solid substrate, to limit diffusion and admixing of the samples. In particular, the libraries may be immobilized to a substantially planar solid phase, including membranes and non-porous substrates such as plastic and glass. Furthermore, the samples are preferably arranged in such a way that indexing (i.e., reference or access to a particular sample) is facilitated. Typically the samples are applied as spots in a grid formation. Common assay systems may be adapted for this purpose. For example, an array may be immobilized on the surface of a microplate, either with multiple samples in a well, or with a single sample in each well. Furthermore, the solid substrate may be a membrane, such as a nitrocellulose or nylon membrane (for example, membranes used in blotting experiments). Alternative substrates include glass, or silica-based substrates. Thus, the samples are immobilized by any suitable method known in the art, for example, by charge interactions, or by chemical coupling to the walls or bottom of the wells, or the surface of the membrane. Other means of arranging and fixing may be used, for example, pipetting, drop-touch, piezoelectric means, ink-jet and bubblejet technology, electrostatic application, etc. In the case of silicon-based chips, photolithography may be utilised to arrange and fix the samples on the chip.

The samples may be arranged by being “spotted” onto the solid substrate; this may be done by hand or by making use of robotics to deposit the sample. In general, arrays may be described as macroarrays or microarrays, the difference being the size of the sample spots. Macroarrays typically contain sample spot sizes of about 300 microns or larger and may be easily imaged by existing gel and blot scanners. The sample spot sizes in microarrays are typically less than 200 microns in diameter and these arrays usually contain thousands of spots. Thus, microarrays may require specialized robotics and imaging equipment, which may need to be custom made. Instrumentation is described generally in a review by Cortese, 2000, The Scientist 14[11]:26.

Techniques for producing immobilized libraries of DNA molecules have been described in the art. Generally, most prior art methods described how to synthesize single-stranded nucleic acid molecule libraries, using for example masking techniques to build up various permutations of sequences at the various discrete positions on the solid substrate. U.S. Pat. No. 5,837,832 describes an improved method for producing DNA arrays immobilized to silicon substrates based on very large scale integration technology. In particular, U.S. Pat. No. 5,837,832 describes a strategy called “tiling” to synthesize specific sets of probes at spatially-defined locations on a substrate which may be used to produced the immobilized DNA libraries of the present invention. U.S. Pat. No. 5,837,832 also provides references for earlier techniques that may also be used. Arrays may also be built using photo deposition chemistry.

To aid detection, labels are typically used—such as any readily detectable reporter, for example, a fluorescent, bioluminescent, phosphorescent, radioactive, etc. reporter. Labelling of probes and targets is also disclosed in Shalon et al., 1996, Genome Res 6(7):639-45.

Examples of DNA arrays include where probe cDNA (500˜5,000 bases long) is immobilized to a solid surface such as glass using robot spotting and exposed to a set of targets either separately or in a mixture. This method is widely considered as having been developed at Stanford University (Ekins and Chu, 1999, Trends in Biotechnology, 1999, 17, 217-218).

Another example of a DNA array is where an array of oligonucleotides (20-25-mer oligos, preferably, 40-60 mer oligos) or peptide nucleic acid (PNA) probes are synthesized either in situ (on-chip) or by conventional synthesis followed by on-chip immobilization. The array is exposed to labelled sample DNA, hybridized, and the identity/abundance of complementary sequences are determined. Such a DNA chip is sold by Affymetrix, Inc., under the GeneChip® trademark. Agilent and Nimblegen also provide suitable arrays (eg. genomic tiling arrays).

In other embodiments, high throughput DNA sequencing promises to become an affordable and more quantitative alternative for microarrays to analyze large collections of DNA sequences. Examples of high-throughput sequencing approaches are listed in E. Y. Chan, Mutation Research 573 (2005) 13-40 and include, but are not limited to, near-term sequencing approaches such as cycle-extension approaches, polymerase reading approaches and exonuclease sequencing, revolutionary sequencing approaches such as DNA scanning and nanopore sequencing and direct linear analysis. Examples of current high-throughput sequencing methods are 454 (pyro)sequencing, Solexa Genome Analysis System, Agencourt SOLiD sequencing method (Applied Biosystems), MS-PET sequencing (Ng et al., 2006, http://nar(dot)oxfordjournals(dot)org/cgi/content/full/34/12/e84).

### Probes

As used herein, the term “probe” refers to a molecule (e.g., an oligonucleotide, whether occurring naturally as in a purified restriction digest or produced synthetically, recombinantly or by PCR amplification), that is capable of hybridizing to another molecule of interest (e.g., another oligonucleotide). When probes are oligonucleotides they may be single-stranded or double-stranded. Probes are useful in the detection, identification and isolation of particular targets (e.g., gene sequences). As described herein, it is contemplated that probes used in the present invention may be labelled with a label so that is detectable in any detection system, including, but not limited to enzyme (e.g., ELISA, as well as enzyme-based histochemical assays), fluorescent, radioactive, and luminescent systems.

With respect to arrays and microarrays, the term “probe” is used to refer to any hybridizable material that is affixed to the array for the purpose of detecting a nucleotide sequence that has hybridized to said probe. Preferably, these probes are 25-60 mers or longer.

The present invention further encompasses probes according to the present invention that are immobilized on a solid or flexible support, such as paper, nylon or other type of membrane, filter, chip, glass slide, microchips, microbeads, or any other such matrix, all of which are within the scope of this invention.

The primers and probes described herein may be readily prepared by, for example, directly synthesizing the fragment by chemical means or by introducing selected sequences into recombinant vectors for recombinant production. Methods for making a vector or recombinants or plasmid for amplification of the fragment either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6,312,683; U.S. patent application Ser. No. 920,197, filed Oct. 16, 1986; WO 90/01543; WO91/11525; WO 94/16716; WO 96/39491; WO 98/33510; EP 265785; EP 0 370 573; Andreansky et al., Proc. Natl. Acad. Sci. USA 1996; 93:11313-11318; Ballay et al., EMBO J. 1993; 4:3861-65; Feigner et al., J. Biol. Chem. 1994; 269:2550-2561; Frolov et al., Proc. Natl. Acad. Sci. USA 1996; 93:11371-11377; Graham, Tibtech 1990; 8:85-87; Grunhaus et al., Sem. Virol. 1992; 3:237-52; Ju et al., Diabetologia 1998; 41:736-739; Kitson et al., J. Virol. 1991; 65:3068-3075; McClements et al., Proc. Natl. Acad. Sci. USA 1996; 93:11414-1.1420; Moss, Proc. Natl. Acad. Sci. USA 1996; 93:11341-11348; Paoletti, Proc. Natl. Acad. Sci. USA 1996; 93:11349-11353; Pennock et al., Mol. Cell. Biol. 1984; 4:399-406; Richardson (Ed), Methods in Molecular Biology 1995; 39, “Baculovirus Expression Protocols,” Humana Press Inc.; Smith et al. (1983) Mol. Cell. Biol. 1983; 3:2156-2165; Robertson et al., Proc. Natl. Acad. Sci. USA 1996; 93:11334-11340; Robinson et al., Sem. Immunol. 1997; 9:271; and Roizman, Proc. Natl. Acad. Sci. USA 1996; 93:11307-11312. Strategies for probe design are described in WO95/11995, EP 717,113 and WO97/29212.

In order to generate data from array-based assays a signal is detected that signifies the presence of or absence of hybridization between a probe and a nucleotide sequence. The present invention further contemplates direct and indirect labelling techniques. For example, direct labelling incorporates fluorescent dyes directly into the nucleotide sequences that hybridize to the array-associated probes (e.g., dyes are incorporated into nucleotide sequence by enzymatic synthesis in the presence of labelled nucleotides or PCR primers). Direct labelling schemes yield strong hybridization signals, typically using families of fluorescent dyes with similar chemical structures and characteristics, and are simple to implement. In some embodiments comprising direct labelling of nucleic acids, cyanine or alexa analogs are utilized in multiple-fluor comparative array analyses. In other embodiments, indirect labelling schemes can be utilized to incorporate epitopes into the nucleic acids either prior to or after hybridization to the microarray probes. One or more staining procedures and reagents are used to label the hybridized complex (e.g., a fluorescent molecule that binds to the epitopes, thereby providing a fluorescent signal by virtue of the conjugation of dye molecule to the epitope of the hybridised species).

Oligonucleotide sequences used as probes according to the present invention may be labeled with a detectable moiety. Various labeling moieties are known in the art. Said moiety may be, for example, a radiolabel (e.g., 3H, 125I, 35S, 14C, 32P, etc.), detectable enzyme (e.g. horse radish peroxidase (HRP), alkaline phosphatase etc.), a fluorescent dye (e.g., fluorescein isothiocyanate, Texas red, rhodamine, Cy3, Cy5, Bodipy, Bodipy Far Red, Lucifer Yellow, Bodipy 630/650-X, Bodipy R6G-X and 5-CR 6G, and the like), a colorimetric label such as colloidal gold or colored glass or plastic (e.g. polystyrene, polypropylene, latex, etc.), beads, or any other moiety capable of generating a detectable signal such as a colorimetric, fluorescent, chemiluminescent or electrochemiluminescent (ECL) signal.

Probes may be labeled directly or indirectly with a detectable moiety, or synthesized to incorporate the detectable moiety. In one embodiment, a detectable label is incorporated into a nucleic acid during at least one cycle of a cyclic polymerase-mediated amplification reaction. For example, polymerases can be used to incorporate fluorescent nucleotides during the course of polymerase-mediated amplification reactions. Alternatively, fluorescent nucleotides may be incorporated during synthesis of nucleic acid primers or probes. To label an oligonucleotide with the fluorescent dye, one of conventionally-known labeling methods can be used (Nature Biotechnology, 14, 303-308, 1996; Applied and Environmental Microbiology, 63, 1143-1147, 1997; Nucleic Acids Research, 24, 4532-4535, 1996). An advantageous probe is one labeled with a fluorescent dye at the 3′ or 5′ end and containing G or C as the base at the labeled end. If the 5′ end is labeled and the 3′ end is not labeled, the OH group on the C atom at the 3′-position of the 3′ end ribose or deoxyribose may be modified with a phosphate group or the like although no limitation is imposed in this respect.

Spectroscopic, photochemical, biochemical, immunochemical, electrical, optical or chemical means can be used to detect such labels. The detection device and method may include, but is not limited to, optical imaging, electronic imaging, imaging with a CCD camera, integrated optical imaging, and mass spectrometry. Further, the amount of labeled or unlabeled probe bound to the target may be quantified. Such quantification may include statistical analysis. In other embodiments the detection may be via conductivity differences between concordant and discordant sites, by quenching, by fluorescence perturbation analysis, or by electron transport between donor and acceptor molecules.

In yet another embodiment, detection may be via energy transfer between molecules in the hybridization complexes in PCR or hybridization reactions, such as by fluorescence energy transfer (FET) or fluorescence resonance energy transfer (FRET). In FET and FRET methods, one or more nucleic acid probes are labeled with fluorescent molecules, one of which is able to act as an energy donor and the other of which is an energy acceptor molecule. These are sometimes known as a reporter molecule and a quencher molecule respectively. The donor molecule is excited with a specific wavelength of light for which it will normally exhibit a fluorescence emission wavelength. The acceptor molecule is also excited at this wavelength such that it can accept the emission energy of the donor molecule by a variety of distance-dependent energy transfer mechanisms. Generally the acceptor molecule accepts the emission energy of the donor molecule when they are in close proximity (e.g., on the same, or a neighboring molecule). FET and FRET techniques are well known in the art. See for example U.S. Pat. Nos. 5,668,648, 5,707,804, 5,728,528, 5,853,992, and 5,869,255 (for a description of FRET dyes), Tyagi et al. Nature Biotech. vol. 14, p 303-8 (1996), and Tyagi et al., Nature Biotech. vol 16, p 49-53 (1998) (for a description of molecular beacons for FET), and Mergny et al. Nucleic Acid Res. vol 22, p 920-928, (1994) and Wolf et al. PNAS vol 85, p 8790-94 (1988) (for general descriptions and methods fir FET and FRET), each of which is hereby incorporated by reference.

The probes for use in an array of the invention may be greater than 40 nucleotides in length and may be isothermal.

In some embodiments, the probes, array of probes or set of probes will be immobilized on a support. Supports (e.g., solid supports) can be made of a variety of materials, such as glass, silica, plastic, nylon or nitrocellulose. Supports are preferably rigid and have a planar surface. Supports typically have from about 1-10,000,000 discrete spatially addressable regions, or cells. Supports having about 10-1,000,000 or about 100-100,000 or about 1000-100,000 cells are common. The density of cells is typically at least about 1000, 10,000, 100,000 or 1,000,000 cells within a square centimeter. In some supports, all cells are occupied by pooled mixtures of probes or a set of probes. In other supports, some cells are occupied by pooled mixtures of probes or a set of probes, and other cells are occupied, at least to the degree of purity obtainable by synthesis methods, by a single type of oligonucleotide.

Arrays of probes or sets of probes may be synthesized in a step-by-step manner on a support or can be attached in presynthesized form. One method of synthesis is VLSIPS™ (as described in U.S. Pat. No. 5,143,854 and EP 476,014), which entails the use of light to direct the synthesis of oligonucleotide probes in high-density, miniaturized arrays. Algorithms for design of masks to reduce the number of synthesis cycles are described in U.S. Pat. No. 5,571,639 and U.S. Pat. No. 5,593,839. Arrays can also be synthesized in a combinatorial fashion by delivering monomers to cells of a support by mechanically constrained flowpaths, as described in EP 624,059. Arrays can also be synthesized by spotting reagents on to a support using an ink jet printer (see, for example, EP 728,520).

### Data Analysis

Data analysis is also an important part of an experiment involving arrays. The raw data from an array experiment typically are images, which need to be transformed into matrices—tables where rows represent, for example, genes, columns represent, for example, various samples such as tissues or experimental conditions, and numbers in each cell for example characterize the expression of a particular sequence (for example, a second sequence that has ligated to the first (target) nucleotide sequence) in the particular sample. These matrices have to be analyzed further, if any knowledge about the underlying biological processes is to be extracted. Methods of data analysis (including supervised and unsupervised data analysis as well as bioinformatics approaches) are disclosed in Brazma and Vilo J (2000) FEBS Lett 480(1):17-24.

### Kits

The materials for use in the methods of the present invention are ideally suited for preparation of kits. Oligonucleotides may be provided in containers that can be in any form, e.g., lyophilized, or in solution (e.g., a distilled water or buffered solution), etc. In one aspect of the present invention, there is provided a kit comprising a set of probes as described herein, an array and optionally one or more labels. In another aspect, there is provided an RT-MLPA kit comprising a set of reverse transcriptase primers as described herein, and appropriate ligases, buffers, and PCR primers. In the kits of the invention, a set of instructions will also typically be included.

The oligonucleotide primers and probes of the present invention have commercial applications in prognostic kits for the detection of the expression level of a gene, such as a MEDIATOR complex and/or SWI/SNF complex gene, in the tumor cells of a patient. A test kit according to the invention may comprise any of the oligonucleotide primers or probes according to the invention. Such a test kit may additionally comprise one or more reagents for use in cyclic polymerase mediated amplification reactions, such as DNA polymerases, nucleotides (dNTPs), buffers, and the like. A kit according to the invention may also include, for example, a lysing buffer for lysing cells contained in the specimen.

A test kit according to the invention may comprise a pair of oligonucleotide primers according to the invention and a probe comprising an oligonucleotide according to the invention. Advantageously, the kit further comprises additional means, such as reagents, for detecting or measuring the binding of the primers and probes of the present invention, and also ideally a positive and negative control.

The invention will now be further described by way of the following non-limiting examples.

### Example 1

### Identification of MED12, ARID1A and SMARCE1 as Molecular Determinants of Resistance to ALK Inhibitors in an EML4-ALK Positive NSCLC Cell Line Using a shRNA Barcode Screen

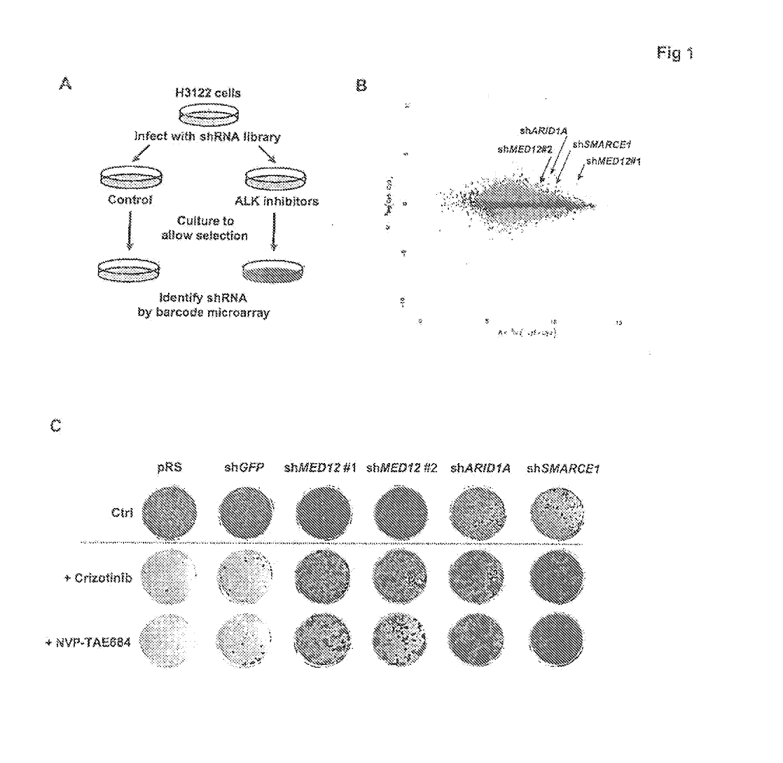

The ALK inhibitors crizotinib and NVP-TAE684 potently inhibit the human NSCLC cell lines that harbor EML4-ALK translocations (Galkin et al., 2007; Koivunen et al., 2008; Soda et al., 2007). The NSCLC cell line H3122 carries the EML4-ALK translocation and is exquisitely sensitive to ALK inhibitors. To identify novel determinants of resistance to ALK inhibitors in NSCLC cell lines, Applicants performed a large-scale RNAi-based loss-of-function genetic screen using a collection of 24,000 short hairpin (shRNA) vectors targeting 8,000 human genes (Berns et al., 2004; Brummelkamp et al., 2002). Applicants used a barcoding technology to identify genes whose suppression causes resistance to ALK inhibitors (Brummelkamp et al., 2006; Holzel et al.). The entire shRNA library was introduced into H3122 cells by retroviral infection and cells were plated at low density with or without ALK inhibitors (FIG. 1A). After four weeks of incubation with ALK inhibitors and the emergence of resistant cell clones, genomic DNA was isolated from treated and untreated cultures. The stably integrated shRNA cassettes (19-mer bar code sequences) were recovered by PCR from genomic DNA. The relative abundance of individual shRNA vectors was quantified by hybridization of the PCR products to microarrays harboring all 24,000 barcode sequences. The barcode screen was carried out in triplicate and the combined results are shown in FIG. 1B. Each dot in the M/A-plot represents one individual shRNA vector in the library. M- and A-values reflect relative enrichment and hybridization signal intensity. Reproducible outliers are generally located in the right upper corner. Low-intensity spots are prone to technical artifacts and thus unreliable. Therefore, Applicants restricted their candidate selection by applying M/A cut-off values of M≧7.5 and A≧7.5 as previously described (Holzel et al.). The identification of independent shRNAs against the same gene or single shRNAs targeting multiple components of the same complex or signaling pathway strongly suggest a genuine hit from the screen. Applying these filter criteria, Applicants identified shRNAs against the genes MED12, ARID1A and SMARCE1.

### MED12, ARID1A and SMARCE1 are Components of Large Multi-Subunit Mediator and SWI/SNF Complexes Involved in Transcriptional Regulation and Chromatin Remodeling

The MED12 gene encodes for a component of the large mediator complex (˜2MDa) that contains at least 33 different subunits and associates with RNA polymerase II at the promoters of genes (Malik and Roeder). Thereby, the Mediator complex is involved in transcriptional regulation. Initially it was thought that the mediator complex is exclusively required for active transcription of genes, but recent studies suggest additional and broader roles in transcriptional regulation, such as epigenetic silencing. In particular, MED12 was implicated in contributing to silencing of neuronal genes in non-neuronal cells by the recruitment of the H3K9 histone methyltransferase EHMT2 (G9a) in a REST dependent manner (Ding et al., 2008). Interestingly, mutations in MED12 are causal for some rare mental retardation syndromes and aberrant gene regulation might contribute to the phenotypic manifestations of these diseases (Risheg et al., 2007; Schwartz et al., 2007). In general, only a few studies have addressed the specific function of individual components of the mediator complex.

ARID1A and SMARCE1 are both components of the SWI/SNF chromatin-remodeling complex (Reisman et al., 2009). The SWI/SNF complex is also a large multi-subunit complex that contains two mutual exclusive but non-redundant subunits with ATPase activity. The ATPases SMARCA2 (BRM1) and SMARCA4 (BRG1) are required for the ATP dependent re-positioning of histones within the chromatin. This ATP-dependent chromatin remodeling activity impacts diverse chromatin related biological processes such as gene transcription and DNA repair. The SWI/SNF complex is conserved throughout evolution from yeast to man. Hence, it is remarkable that several subunits of the SWI/SNF complex have been identified as tumor suppressors. Deletions of SMARCB1 (INI1, BAF47) are found in malignant rhabdoid tumors, a highly aggressive childhood cancer (Versteege et al., 1998). Inactivating truncating mutations of ARID1A and PBRM1 were found in more than 50% and 40% of clear cell ovarian and renal cancer, respectively (Jones et al.; Varela et al.). SMARCA4 (BRG1) is frequently mutated in NSCLC cell lines, but also in primary tumors (Medina et al., 2008; Rodriguez-Nieto et al.). In conclusion, there is substantial evidence in the literature that specific components of the SWI/SNF complex function as tumor suppressors in a tumor type dependent manner, but the molecular basis of this selectivity remains unknown.

Validation of shRNA Barcode Screen Results

To validate the results of their screen, Applicants individually introduced the respective knockdown vectors from the NKI shRNA library against MED12 (#1 and #2), ARID1A and SMARCE1 into H3122 cells by retroviral infections and confirmed that all four shRNA vectors confer resistance to the ALK inhibitors crizotinib and NVP-TAE684 in H3122 cells (FIG. 1C). To rule out ‘off-target’ effects, a common problem in the field of RNAi screening, Applicants only consider a gene identified from the screen as a genuine hit, if at least two independent shRNAs suppress the expression of the target mRNA and also confer resistance to the ALK inhibitors (Echeverri et al., 2006). In particular, Applicants considered a gene identified in the screen as a genuine hit, if at least two independent shRNAs suppress the expression of the target and also confer crizotinib resistance. Only one gene fulfilled these criteria: MED12, encoding a component of the large MEDIATOR transcriptional adapter complex.

To validate MED12 as a gene whose suppression confers resistance to crizotinib, Applicants individually introduced the two MED12 shRNA vectors (#1 and #2) from the library and one newly generated shRNA (#3) into H3122 cells by retroviral infection. Empty vector (pRS) or shRNA targeting GFP (shGFP) served as controls throughout the study. All three distinct MED12 knockdown vectors conferred resistance to both crizotinib and the second ALK inhibitor NVP-TAE684 in long-term colony formation assays (FIG. 3A) and also efficiently suppressed MED12 mRNA and protein expression (FIGS. 3B, 3C). Similarly, expression of additional independent lentiviral shMED12 vectors (#4 and #5) in H3122 cells also conferred resistance to ALK inhibitors (FIG. 4A-C and data not shown). Furthermore, reconstitution of Med12 in MED12 knockdown (MED12KD) H3122 cells by introducing a RNAi-resistant mouse Med12 cDNA restored the sensitivity of these cells to ALK inhibition (FIG. 4A). Applicants confirmed that the reconstituted MED12/Med12 total proteins in MED12KD cells were at physiological levels similar to parental cells (FIG. 4B), and that knockdown of human MED12 mRNA was maintained in cells expressing both human shMED12 vectors and the mouse Med12 cDNA (FIG. 4C, D). Together, these results validate MED12 as a genuine on target hit and establish its role in resistance to ALK inhibition.

Next, Applicants validated that ARID1A and SMARCE1 are on-target hits causally involved in the resistance to ALK inhibitors. As Applicants have only identified single shRNAs (shARID1A#1, shSMARCE1#1) against these genes from the barcode screen, they generated additional non-overlapping shRNAs against ARID1A and SMARCE1 (shARID1A#2, shSMARCE1#2) and introduced them into H3122 cells by retroviral infection. The independent shRNAs recapitulated the resistance to ALK inhibitors (FIG. 5A). It is noteworthy that knockdown of either ARID1A or SMARCE1 impaired proliferation of H3122 cells in the absence of the inhibitors. Applicants confirmed the suppression of ARID1A and SMARCE1 mRNA and protein levels by qRT-PCR and immunoblotting (FIG. 5B-5E). Again, these results show that ARID1A and SMARCE1 are genuine on-target hits from the screen.

Next, Applicants introduced silent mutations into a human SMARCE1 cDNA expression construct and thereby generated two separate shRNA resistant (non-degradable, ND) forms of SMARCE1 (SMARCE1-ND) that cannot be targeted by shSMARCE1#1 and shSMARCE1#2. H3122 cells stably infected with pRS, shSMARCE1#1 or #2 were super-infected with retroviral expression constructs encoding for the respective non-degradable forms of SMARCE1 or the pMx empty control vector. Reconstitution of SMARCE1 restored sensitivity of SMARCE1 knockdown cells to ALK inhibitors (FIG. 6A). Applicants confirmed reconstituted SMARCE1 protein levels in SMARCE1 knockdown cells by immunoblotting using an SMARCE1 specific antibody, again achieving close to endogenous level of SMARCE1 (FIG. 6B). Applicants also verified a persistent knockdown of the endogenous human SMARCE1 mRNA in cells expressing the non-degradable SMARCE1 cDNAs by qRT-PCR using a human SMARCE1 3′UTR specific primer pair (FIG. 6C). In turn, Applicants also confirmed expression of the SMARCE1 cDNA using an open reading frame specific primer pair detecting endogenous and ectopic (total) SMARCE1 (FIG. 6D). In summary, these experiments demonstrate that SMARCE1 is a genuine on-target hit from the ALK inhibitor shRNA resistance screen.

### MED12, ARID1A and SMARCE1 are Molecular Determinants of Resistance to Tyrosine Kinase Inhibitors in Multiple NSCLC Cell Lines

Next, Applicants addressed the context dependency of their findings by studying independent NSCLC cell lines. The RAS/PI3K signaling cascade is a common denominator of all activated tyrosine kinases in NSCLC such as the EGFR (Pao and Chmielecki). Therefore, Applicants hypothesized that loss of MED12, SMARCE1 and ARID1A might also confer resistance to other tyrosine kinase inhibitors in cell lines that harbor respective activating mutations or amplifications.

NSCLC with activating mutations of the EGFR can be effectively treated with the EGFR inhibitors gefitinib and erlotinib. Several NSCLC cell lines with EGFR mutations (PC9, H3255) were identified that are exquisitely sensitive to gefitinib and erlotinib at low nanomolar concentrations. Applicants introduced MED12 specific shRNAs (shMED12_TRC#3 and #5) into PC9 cells (EGFR^{delE746-A750}). Suppression of MED12 rendered PC9 cells insensitive to the EGFR inhibitor gefitinib (FIG. 7A, left panel). In addition, reconstitution of PC9 MED12-knockdown cells with the mouse Med12 cDNA restored their sensitivity to gefitinib (FIG. 7A, right panel). Using an antibody that recognizes human and mouse MED12/Med12, Applicants confirmed the suppression and restoration of MED12 protein level in the indicated PC9 cell lines by immunoblotting (FIG. 7B). Applicants also verified persistent knockdown of endogenous MED12 by qRT-PCR using a human MED12 specific primer pair (FIG. 7C). Likewise, Applicants controlled the ectopic expression of the mouse Med12 cDNA by qRT-PCR using a mouse Med12 specific primer pair (FIG. 7D). Furthermore, H3255 (EGFR^{L858R}) cells were stably infected with three MED12 shRNA or control constructs (pRS and shGFP) and incubated with two EGFR inhibitors (gefitinib and erlotinib). Control cells were effectively eradicated, whereas shMED12 cells were insensitive to the treatment with the inhibitors (FIG. 8A). Applicants confirmed suppression of MED12 by qRT-PCR (FIG. 8B). In conclusion, Applicants demonstrated that loss of MED12 confers resistance to ALK and EGFR tyrosine kinase inhibitors in multiple NSCLC cell lines.

Next, Applicants asked whether ARID1A determines sensitivity to tyrosine kinase inhibitors in multiple NSCLC cell lines (context dependency). Applicants introduced the retroviral shRNA vectors against ARID1A (#1 and #2) or control vectors (pRS and shGFP) into PC9 (EGFR^{delE746-A750}) and H1993 (MET-amplified) cells (FIGS. 1A and 1C). Suppression of ARID1A conferred resistance to the EGFR inhibitor gefitinib and the MET inhibitor crizotinib in PC9 and H1993 cells, respectively. Knockdown of ARID1A mRNA was confirmed by qRT-PCR (FIGS. 3B and 3D).

Now, Applicants addressed whether SMARCE1 is also determinant of tyrosine kinase inhibitor sensitivity in multiple NSCLC cell lines (context dependency). PC9 (EGFR^{delE746-A750}), H1993 (MET-amplified) and EBC-1 (MET-amplified) cells were stably infected with the retroviral shRNA constructs pRS, shSMARCE1#1 and #2 and were treated with the EGFR inhibitor geftitinib (PC9) or MET inhibitor crizotinib (H1993, EBC1). In all cases, suppression of SMARCE1 conferred resistance to the respective inhibitors (FIG. 10A, 11A and 12A, left panels). In parallel, the PC9, H1993 and EBC-1 cells expressing shSMARCE1#1 and #2 were infected with retroviral expression constructs encoding for the non-degradable forms of SMARCE1 (SMARCE1-ND). Reconstitution of SMARCE1 restored the sensitivity of SMARCE1-knockdown cells to the EGFR inhibitor geftitinib or MET inhibitor crizotinib (FIG. 10A, 11A and 12A, right panels). Applicants confirmed reconstituted SMARCE1 protein levels in SMARCE1-knockdown cells by immunoblotting using an SMARCE1 specific antibody, again achieving close to endogenous level of SMARCE1 in most of the cases (FIG. 10B, 11B and 12B). Applicants also verified a persistent knockdown of the endogenous human SMARCE1 mRNA in cells expressing the non-degradable SMARCE1 cDNAs by qRT-PCR using a human SMARCE1 3′UTR specific primer pair (FIG. 10C, 11C and 12C). In turn, Applicants also confirmed expression of the non-degradable SMARCE1 cDNAs using an open reading frame specific primer pair detecting endogenous and ectopic (total) SMARCE1 (FIG. 10D, 11D and 12D). It has been shown that excess SMARCE1 protein is rapidly degraded by the proteasome, suggesting that SMARCE1 protein stability requires incorporation into the SWI/SNF complex. This finding is in line with Applicants' observations from the reconstitution experiments that the protein levels of the non-degradable forms SMARCE1 were close to endogenous SMARCE1 protein level despite a significant mRNA overexpression. In conclusion, SMARCE1 is a determinant of resistance to tyrosine kinase inhibitors in multiple NSCLC cell lines.

### the Role of RAS-GAPs in the Control of Tyrosine Kinase Inhibitor Sensitivity in NSCLC Cell Lines

Constitutive signaling from mutated receptor tyrosine kinases such EGFR leads to activation of the RAS small GTP-binding proteins (KRAS, HRAS, NRAS). In particular KRAS is one of the most frequently mutated genes in a variety of cancers including NSCLC. RAS mutations impair the intrinsic GTPase activity and therefore prevent the conversion of active GTP-bound form into the inactive GDP-bound form (Karnoub and Weinberg, 2008). Introduction of constitutive active alleles of RAS in NSCLC cell lines renders the insensitive to tyrosine kinase inhibitors (data not shown). Therefore, inhibition of RAS is key mechanism of the efficacy of tyrosine kinase inhibitors. Applicants reasoned that direct negative regulators of RAS proteins might be critical determinants of sensitivity to tyrosine kinase inhibitors in NSCLC cell lines. The human genome encodes for 14 putative RAS-GTPase activating proteins (RAS-GAPs) that stimulate the GTPase activity of RAS proteins and promote the conversion of active GTP-loaded RAS into the inactive GDP-loaded form (Bernards, 2003). Applicants retrieved shRNAs covering the 14 putative human RAS-GAPs from the TRC shRNA collection and all shRNAs targeting the same gene were pooled together. Applicants infected PC9 cells with the 14 RAS-GAP pools in addition to the control vectors pLKO and shGFP. The cells were plated at low density and treated with the two EGFR inhibitors gefitinib and erlotinib or left untreated (FIG. 13). Several RAS-GAP pools conferred resistance to the EGFR inhibitors in the PC9 cell lines: Applicants observed the strongest resistance phenotype for the pool targeting the RAS-GAP DAB2IP. The pools directed against NF1 and RASAL3 also rendered the cells less sensitive to both EGFR inhibitors, whereas the pools targeting RASA2 exhibited inconsistent results.

First, Applicants focused on the RAS-GAPs DAB2IP and NF1. NF1 is bona-fide tumor suppressor mutated in several cancers and also causal for the hereditable disease neurofibromatosis type I, a benign tumor syndrome with strong predisposition to several malignant cancers (Cichowski and Jacks, 2001). DAP2IP plays an important role in prostate cancer and loss of its expression is associated with an aggressive metastatic disease (Min et al.). To validate the results of Applicants' focused shRNA mini-screen, Applicants individually introduced the five DAB2IP shRNAs from the TRC shRNA collection into PC9 cells (FIG. 14A). Applicants noticed that shDAB2IP#2 and to a lesser extent shDAB2IP#5 exhibited toxicity. Applicants assume that this toxicity is unrelated to the suppression of DAB2IP, as shDAB2IP#5 failed to induce a knockdown of DAB2IP. The two best shRNA vectors (shDAB2IP#1 and #3) conferred resistance to the EGFR inhibitors gefitinib and erlotinib. Suppression of DAB2IP mRNA levels was confirmed by qRT-PCR (FIG. 14B). Next, Applicants addressed whether loss of DAB2IP affects the activity of downstream signaling components of the RAS pathway, in particular the phosphorylation (activation) status of ERK. Total cell lysates were prepared from control and shDAB2IP cells (PC9) in the absence or presence of gefitinib (FIG. 14C). Applicants confirmed suppression of DAB2IP protein level in shDAB2IP expressing cells. Consistent with the inhibition of RAS by RAS-GAPs, Applicants observed elevated levels of phospho-ERK in shDAB2IP cells indicating hyperactivation of downstream components of the RAS signaling cascade. Importantly, phosphorylation of ERK was maintained in shDAB2IP cells treated with gefitinib being in line with resistance to EGFR inhibitors in the colony formation assays. Next, Applicants individually introduced the five NF1 shRNAs from the TRC shRNA collection into PC9 cells (FIG. 15A). The two best shRNA vectors (shNF1#2 and #5) conferred resistance to the EGFR inhibitors gefitinib and erlotinib. Suppression of NF1 mRNA and protein levels was confirmed by qRT-PCR and immunoblotting (FIGS. 15B and 15C). Applicants' results show that the DAB2IP and NF1 are important determinant of sensitivity NSCLC cell to EGFR inhibitors.

### Suppression of MED12 and SMARCE1 Leads to Activation of ERK Signaling in NSCLC Cells.

Given that loss of MED12 or SMARCE1 causes resistance to multiple tyrosine kinase inhibitors in NSCLC cell lines, Applicants asked whether the activity of downstream components of receptor tyrosine kinase signaling is altered. ERK is a key downstream component and its phosphorylation status positively correlates with its activation that can be determined by specific antibodies against the phosphorylated form of ERK. H3122 cells were infected with two independent controls shRNA vectors or shRNAs targeting either MED12 or SMARCE1 and confirmed loss of MED12 or SMARCE1 protein by immunoblotting (FIGS. 16A and B). The cells were also treated of left untreated with the ALK inhibitor NVP-TAE684, to address the activation status of ERK in the presence or absence of the inhibitor. Interestingly, H3122 MED12 knockdown cells maintained higher levels of ERK phosphorylation in the presence of the inhibitor (FIG. 16A). Loss of SMARCE1 resulted in an increased ERK activation even in the absence of the inhibitor and consistently maintained higher levels of phosphorylated ERK in the presence of NVP-TAE684 (FIG. 16B). In conclusion, elevated activation of the key downstream component ERK upon suppression of MED12 or SMARCE1 is consistent with resistance to upstream inhibition by tyrosine kinase inhibitors. Further, Applicants could also show that loss of MED12 resulted in elevated levels of ERK phosphorylation and hence activation in PC9 cells (FIG. 16C). Applicants conclude that MED12 and SMARCE1 regulate ERK activation in multiple NSCLC lung cancer cell lines. Accordingly, in certain embodiments, MED12 and/or SMARCE1 expression and/or mutation status is an important determinant of treatment responses to tyrosine kinase inhibitors in the clinic.

### MED12 Loss Leads to ERK Activation and Multi Targeted-Drug Resistance in Different Cancer Types

Applicants' finding that MED12 suppression confers resistance to both ALK and EGFR inhibitors in NSCLCs suggests that MED12 might act on a critical pathway downstream of both ALK and EGFR. As pointed out above, RAS signaling is downstream of all activated RTKs in NSCLC (Pao and Chmielecki, 2010). Applicants first asked which components of the RAS pathway could cause resistance to RTK inhibition in H3122 and PC9 cells by expressing active alleles of these genes (FIG. 31). As expected, activation of RAS signaling by expression of KRASV12 conferred resistance to upstream inhibition by TKIs targeting ALK and EGFR (FIG. 31). BRAFV600E and MEK-DD also conferred resistance to TKIs, but PIK3CAH1047R, RALAQ75L and RALBQ72L failed to do so in both cell systems used. These results indicate that activation of the RAS-RAF-MEK cascade is sufficient to cause resistance to ALK and EGFR inhibitors. Applicants therefore asked whether the activity of RAF-MEK-ERK is altered in MED12KD cells. Indeed, H3122 cells expressing shMED12 vectors maintained higher levels of phosphorylated ERK (p-ERK) in the presence of ALK inhibitor (FIG. 17A). Similarly, knockdown of MED12 in PC9 and H3255 cells leads to higher levels of p-ERK in both absence and presence of EGFR inhibitors (FIG. 17B and data not shown). These findings suggest that MED12 loss confers resistance to ALK and EGFR inhibitors in NSCLCs by enhancing ERK activation.

If suppression of MED12 leads to ERK activation, one would expect that MED12 loss might also confer resistance to other cancer drugs targeting the MAPKs upstream of ERK. The small molecule drug PLX4032 (vemurafenib) has proven to be very effective in the treatment of melanoma with BRAFV600E mutations and the MEK inhibitor AZD6244 (seluteminib) is being tested in the clinical trials for the treatment of several cancers. A375 melanoma cells harboring the BRAFV600E mutation are highly sensitive to PLX4032 and AZD6244. Consistent with Applicants' observations made in NSCLC models, Applicants found that suppression of MED12 in A375 cells caused ERK activation (FIG. 17D) and conferred potent resistance to both PLX4032 and AZD6244 (FIG. 17C). Similar results were obtained in an additional melanoma cell line SK-MEL-28 (FIG. 18C, D). SK-CO-1 colorectal cancer (CRC) cells harbor a KRASV12 mutation and are highly sensitive to MEK inhibition by AZD6244. Knockdown of MED12 also resulted in activation of ERK (FIG. 17F) and conferred resistance to AZD6244 in SKCO-1 cells (FIG. 17E). Identical results were observed in the CRC cell line SW1417 harboring a BRAFV600E mutation (FIG. 18E, F).

To extend their findings even further, Applicants asked whether MED12 also confers resistance to a class of multi-kinase inhibitors. Sorafenib targets multiple tyrosine kinases and RAF kinases and is used clinically to treat advanced renal cell carcinoma and hepatocellular carcinoma (HCC). HCC Huh-7 cells are sensitive to sorafenib, but became resistant after knockdown of MED12 (FIG. 17G, H). Taken together, Applicants' data demonstrate that MED12 loss leads to ERK activation and confers resistance to a range of targeted cancer drugs that act upstream of the ERK kinases. Applicants also note that the effects of MED12 suppression appear to be mostly context-independent as its consequences are readily apparent in several major cancer types including NSCLC, melanoma, CRC and HCC.

### Results Melanoma:

### Suppression of MED12 Confers Drug Resistance to BRAF and MEK Inhibitors in BRAF^{V600E} Melanoma Cells

As a first step in expanding Applicants' finding in NSCLC, they examined the potential role of MED12 in drug responses to BRAF and MEK inhibitors in BRAF^{V600E} melanomas where activation of ERK is a common feature of resistant tumors. Since MED12 knockdown leads to higher levels of ERK phosphorylation in NSCLC cells, Applicants asked if MED12 is also critical for drug responses to BRAF and MEK inhibitors in BRAF^{V600E} melanoma cells. A375 (BRAF^{V600E}) melanoma cells stably expressing the retroviral shRNA constructs pRS, shGFP, shSMARCE1#1 and #2 were treated with the BRAF^{V600E} inhibitor PLX4720 or MEK inhibitor PD-0325901. In all cases, suppression of MED12 conferred resistance to the respective inhibitors (FIG. 19).

In addition, Applicants observed similar effects in the melanoma cell line, SK-MEL-28, which expresses BRAF^{V600E}. In particular, Applicants demonstrate that downregulation of MED12 induces resistance to the BRAF inhibitor, PLX 4032, in SK-MEL-28 cells.

### REFERENCES

Bernards, A. (2003). GAPs galore! A survey of putative Ras superfamily GTPase activating proteins in man and Drosophila. Biochim Biophys Acta 1603, 47-82.
Berns, K., Hijmans, E. M., Mullenders, J., Brummelkamp, T. R., Velds, A., Heimerikx, M., Kerkhoven, R. M., Madiredjo, M., Nijkamp, W., Weigelt, B., et al. (2004). A large-scale RNAi screen in human cells identifies new components of the p53 pathway. Nature 428, 431-437.
Brummelkamp, T. R., Bernards, R., and Agami, R. (2002). A system for stable expression of short interfering RNAs in mammalian cells. Science 296, 550-553.
Brummelkamp, T. R., Fabius, A. W., Mullenders, J., Madiredjo, M., Velds, A., Kerkhoven, R. M., Bernards, R., and Beijersbergen, R. L. (2006). An shRNA barcode screen provides insight into cancer cell vulnerability to MDM2 inhibitors. Nat Chem Biol 2, 202-206.
Choi, Y. L., Soda, M., Yamashita, Y., Ueno, T., Takashima, J., Nakajima, T., Yatabe, Y., Takeuchi, K., Hamada, T., Haruta, H., et al. (2010). EML4-ALK mutations in lung cancer that confer resistance to ALK inhibitors. N Engl J Med 363, 1734-1739.
Cichowski, K., and Jacks, T. (2001). NF1 tumor suppressor gene function: narrowing the GAP. Cell 104, 593-604.
Ding, N., Zhou, H., Esteve, P. O., Chin, H. G., Kim, S., Xu, X., Joseph, S. M., Friez, M. J., Schwartz, C. E., Pradhan, S., et al. (2008). Mediator links epigenetic silencing of neuronal gene expression with x-linked mental retardation. Mol Cell 31, 347-359.
Echeverri, C. J., Beachy, P. A., Baum, B., Boutros, M., Buchholz, F., Chanda, S. K., Downward, J., Ellenberg, J., Fraser, A. G., Hacohen, N., et al. (2006). Minimizing the risk of reporting false positives in large-scale RNAi screens. Nat Methods 3, 777-779.
Engelman, J. A., Zejnullahu, K., Mitsudomi, T., Song, Y., Hyland, C., Park, J. O., Lindeman, N., Gale, C. M., Zhao, X., Christensen, J., et al. (2007). MET amplification leads to gefitinib resistance in lung cancer by activating ERBB3 signaling. Science 316, 1039-1043.
Galkin, A. V., Melnick, J. S., Kim, S., Hood, T. L., Li, N., Li, L., Xia, G., Steensma, R., Chopiuk, G., Jiang, J., et al. (2007). Identification of NVP-TAE684, a potent, selective, and efficacious inhibitor of NPM-ALK. Proc Natl Acad Sci USA 104, 270-275.
Hammerman, P. S., Janne, P. A., and Johnson, B. E. (2009). Resistance to Epidermal Growth Factor Receptor Tyrosine Kinase Inhibitors in Non-Small Cell Lung Cancer. Clin Cancer Res 15, 7502-7509.
Holzel, M., Huang, S., Koster, J., Ora, I., Lakeman, A., Caron, H., Nijkamp, W., Xie, J., Callens, T., Asgharzadeh, S., et al. (2010). NF1 is a tumor suppressor in neuroblastoma that determines retinoic acid response and disease outcome. Cell 142, 218-229.
Jackman, D., Pao, W., Riely, G. J., Engelman, J. A., Kris, M. G., Janne, P. A., Lynch, T., Johnson, B. E., and Miller, V. A. (2010). Clinical definition of acquired resistance to epidermal growth factor receptor tyrosine kinase inhibitors in non-small-cell lung cancer. J Clin Oncol 28, 357-360.
Jones, S., Wang, T. L., Shih Ie, M., Mao, T. L., Nakayama, K., Roden, R., Glas, R., Slamon, D., Diaz, L. A., Jr., Vogelstein, B., et al. (2010). Frequent mutations of chromatin remodeling gene ARID1A in ovarian clear cell carcinoma. Science 330, 228-231.
Karnoub, A. E., and Weinberg, R. A. (2008). Ras oncogenes: split personalities. Nat Rev Mol Cell Biol 9, 517-531.
Kobayashi, S., Boggon, T. J., Dayaram, T., Janne, P. A., Kocher, O., Meyerson, M., Johnson, B. E., Eck, M. J., Tenen, D. G., and Halmos, B. (2005). EGFR mutation and resistance of non-small-cell lung cancer to gefitinib. N Engl J Med 352, 786-792.
Koivunen, J. P., Mermel, C., Zejnullahu, K., Murphy, C., Lifshits, E., Holmes, A. J., Choi, H. G., Kim, J., Chiang, D., Thomas, R., et al. (2008). EML4-ALK fusion gene and efficacy of an ALK kinase inhibitor in lung cancer. Clin Cancer Res 14, 4275-4283.
Kwak, E. L., Bang, Y. J., Camidge, D. R., Shaw, A. T., Solomon, B., Maki, R. G., Ou, S. H., Dezube, B. J., Janne, P. A., Costa, D. B., et al. (2010). Anaplastic lymphoma kinase inhibition in non-small-cell lung cancer. N Engl J Med 363, 1693-1703.
Lynch, T. J., Bell, D. W., Sordella, R., Gurubhagavatula, S., Okimoto, R. A., Brannigan, B. W., Harris, P. L., Haserlat, S. M., Supko, J. G., Haluska, F. G., et al. (2004). Activating mutations in the epidermal growth factor receptor underlying responsiveness of non-small-cell lung cancer to gefitinib. N Engl J Med 350, 2129-2139.
Maemondo, M., Inoue, A., Kobayashi, K., Sugawara, S., Oizumi, S., Isobe, H., Gemma, A., Harada, M., Yoshizawa, H., Kinoshita, I., et al. (2010). Gefitinib or chemotherapy for non-small-cell lung cancer with mutated EGFR. N Engl J Med 362, 2380-2388.
Malik, S., and Roeder, R. G. (2010) The metazoan Mediator co-activator complex as an integrative hub for transcriptional regulation. Nat Rev Genet. 11, 761-772.
Medina, P. P., Romero, O. A., Kohno, T., Montuenga, L. M., Pio, R., Yokota, J., and Sanchez-Cespedes, M. (2008). Frequent BRG1/SMARCA4-inactivating mutations in human lung cancer cell lines. Hum Mutat 29, 617-622.
Min, J., Zaslavsky, A., Fedele, G., McLaughlin, S. K., Reczek, E. E., De Raedt, T., Guney, I., Strochlic, D. E., Macconaill, L. E., Beroukhim, R., et al. (2010). An oncogene-tumor suppressor cascade drives metastatic prostate cancer by coordinately activating Ras and nuclear factor-kappaB. Nat Med 16, 286-294.
Pao, W., and Chmielecki, J. (2010). Rational, biologically based treatment of EGFR-mutant non-small-cell lung cancer. Nat Rev Cancer 10, 760-774.
Reisman, D., Glaros, S., and Thompson, E. A. (2009). The SWI/SNF complex and cancer. Oncogene 28, 1653-1668.
Risheg, H., Graham, J. M., Jr., Clark, R. D., Rogers, R. C., Opitz, J. M., Moeschler, J. B., Peiffer, A. P., May, M., Joseph, S. M., Jones, J. R., et al. (2007). A recurrent mutation in MED12 leading to R961W causes Opitz-Kaveggia syndrome. Nat Genet. 39, 451-453.
Rodriguez-Nieto, S., Canada, A., Pros, E., Pinto, A. I., Torres-Lanzas, J., Lopez-Rios, F., Sanchez-Verde, L., Pisano, D. G., and Sanchez-Cespedes, M. (2010). Massive parallel DNA pyrosequencing analysis of the tumor suppressor BRG1/SMARCA4 in lung primary tumors. Hum Mutat.
Rosell, R., Moran, T., Queralt, C., Porta, R., Cardenal, F., Camps, C., Majem, M., Lopez-Vivanco, G., Isla, D., Provencio, M., et al. (2009). Screening for epidermal growth factor receptor mutations in lung cancer. N Engl J Med 361, 958-967.
Rudin, C. M., Avila-Tang, E., Harris, C. C., Herman, J. G., Hirsch, F. R., Pao, W., Schwartz, A. G., Vahakangas, K. H., and Samet, J. M. (2009). Lung cancer in never smokers: molecular profiles and therapeutic implications. Clin Cancer Res 15, 5646-5661.
Schwartz, C. E., Tarpey, P. S., Lubs, H. A., Verloes, A., May, M. M., Risheg, H., Friez, M. J., Futreal, P. A., Edkins, S., Teague, J., et al. (2007). The original Lujan syndrome family has a novel missense mutation (p.N1007S) in the MED12 gene. J Med Genet. 44, 472-477.
Sharma, S. V., Bell, D. W., Settleman, J., and Haber, D. A. (2007). Epidermal growth factor receptor mutations in lung cancer. Nat Rev Cancer 7, 169-181.
Sharma, S. V., and Settleman, J. (2007). Oncogene addiction: setting the stage for molecularly targeted cancer therapy. Genes Dev 21, 3214-3231.
Soda, M., Choi, Y. L., Enomoto, M., Takada, S., Yamashita, Y., Ishikawa, S., Fujiwara, S., Watanabe, H., Kurashina, K., Hatanaka, H., et al. (2007). Identification of the transforming EML4-ALK fusion gene in non-small-cell lung cancer. Nature 448, 561-566.
Varela, I., Tarpey, P., Raine, K., Huang, D., Ong, C. K., Stephens, P., Davies, H., Jones, D., Lin, M. L., Teague, J., et al. (2011). Exome sequencing identifies frequent mutation of the SWI/SNF complex gene PBRM1 in renal carcinoma. Nature 469, 539-542.
Versteege, I., Sevenet, N., Lange, J., Rousseau-Merck, M. F., Ambros, P., Handgretinger, R., Aurias, A., and Delattre, O. (1998). Truncating mutations of hSNF5/INI1 in aggressive paediatric cancer. Nature 394, 203-206.

### Experimental Procedures

shRNA Barcode Screen

The human NKI shRNA library and the barcode screen were performed as described (Berns et al., 2004; Brummelkamp et al., 2006). Additional details can be found at http://www(dot)screeninc(dot)nki(dot)nl.

### Cell Proliferation Assays

Single cell suspensions of the lung cancer cell lines were seeded into 6-well plates (2×10⁴ cells/well) and cultured both in the absence and presence of the ALK inhibitors. At the endpoints of colony formation assays, cells were fixed with formaldehyde, stained with crystal violet (0.1% w/v) and photographed. All relevant assays were performed independently at least three times. All knockdown and overexpression experiments were done by retroviral or lentiviral infections.

### Cell Culture and Viral Transduction

H3122, PC9, H1993, EBC-1, H3255, SK-CO-1, and SW1417 cells were cultured in RPMI with 8% heat-inactivated fetal bovine serum, penicillin and streptomycin at 5% CO₂. 293T, Phoenix cells, A375, SK-MEL-28, and Huh-7 cells were cultured in DMEM with 8% heat-inactivated fetal bovine, serum, penicillin and streptomycin at 5% CO₂. Subclones of each NSCLC cell line expressing the murine ecotropic receptor were generated and used for all experiments shown. Retroviral infections were performed using Phoenix cells as producers of retroviral supernatants using 2.5-3 μg of plasmid DNA as described (http://www(dot)stanford(dot)edu/group/nolan/retroviral systems/phx(dot)html). 293T cells were used as producers of lentiviral supernatants by co-transfecting 3^{rd} generation lentiviral 15 packaging constructs (2 μg of plasmid DNA) along with the pLKO shRNA vectors (2 μg of plasmid DNA). For transfections of 293T cells, Applicants seeded 1.8×10⁶ cells in a 6-well dish in the morning and transfected the cells 6-8 hours later. For transfections of Phoenix cells, Applicants seeded 1.0×10⁶ cells in a 6-well dish in the morning and transfected the cells 6-8 hours later. Cells were refreshed the next day in the morning and afternoon. Viral supernatant was harvested the day thereafter for infections of the target cells. The calcium phosphate method was used for the transfection of Phoenix and 293T cells. Infected NSCLC cells were selected for successful retroviral integration using 2 μg/ml of puromycin.

### Reagents and Antibodies

Crizotinib (S1068), NVP-TAE648 (S1108), gefitinib (S1025), erlotinib (S1023), PLX4032 (S1267) and AZD6244 (S1008) were purchased from Selleck Chemicals. TRC human genome-wide shRNA collection (TRC-Hs1.0) was purchased from Open Biosystems (Huntsville, USA). Further information is available at http://www(dot)broad(dot)mit(dot)edu/genome bio/trc/rnai(dot)html. Antibody against MED12 (A300-774A), SMARCE1 (A300-810A), DAB2IP (A302-439A) and NF1 (A300-140A) was from Bethyl Laboratories; antibody against Vimentin (RV202) was from Abcam; antibody against N-cadherin (ab18203) was from Cell Signaling; antibodies against NF1 (SC-67), HSP90 (H-14), p-ERK (E-4), ERK1 (C-16), ERK2 (C-14), CDK8 (D-9), Lamin A/C (636), SP1 (PEP2) and α-TUBULIN(H-183) were from Santa Cruz Biotechnology; The antibody against ARID1A (H00008289-M01) was from Abnova. A mixture of ERK1 and ERK2 antibodies was used for detection of total ERK.

### Plasmids

All retroviral shRNA vectors were generated by ligating synthetic oligonucleotides (Invitrogen) against the target genes into in the pRetroSuper (pRS) retroviral vector as described (Brummelkamp et al., 2002). The following RNAi target sequences were used for this study.

| | |
|---|---|
| shGFP | GCTGACCCTGAAGTTCATC |
| shMED121#1 | GTACCATGACTCCAATGAG |
| shMED12#2 | GGAAGAGGTGTTTGGGTAC |
| shMED12#3 | GGAGGAACTGCTTGTGCAC |
| shARID1A#1 | GGGGTGAGCTGCAACAAAG |
| shARID1A#2 | AGGAGAAGCTGATCAGTAA |
| shSMARCE1#1 | GGAGAACCGTACATGAGCA |
| shSMARCE1#2 | GGAAGAAAGTCGACAGAGA |

All lentiviral shRNA vectors (TRCN number) were retrieved from the arrayed human TRC shRNA library. Additional information about the shRNA vectors can be found at http://www.broadinstitute.org/rnai/public/clone/search using the TRCN number.

| pLKO_ | | |
|---|---|---|
| control | | No hairpin insert |
| shGFP | | GCAAGCTGACCCTGAAGTTCA |
| shMED12_ | TRCN0000018574 | GCAGCATTATTGCAGAGAAAT |
| TRC#1 | | |
| shMED12_ | TRCN0000018575 | GCTGTTCTCAAGGCTGTGTTT |
| TRC#2 | | |
| shMED12_ | TRCN0000018576 | CGGGTACTTCATACTTTGGAA |
| TRC#3 | | |
| shMED12_ | TRCN0000018577 | GCAGTTCATCTTCGACCTCAT |
| TRC#4 | | |
| shMED12_ | TRCN0000018578 | GCAGAGAAATTACGTTGTAAT |
| TRC#5 | | |
| shNF1_ | TRCN0000039713 | CCATGTTGTAATGCTGCACTT |
| TRC#1 | | |
| shNF1_ | TRCN0000039714 | GCCAACCTTAACCTTTCTAAT |
| TRC#2 | | |
| shNF1_ | TRCN0000039715 | CCTCACAACAACCAACACTTT |
| TRC#3 | | |
| shNF1_ | TRCN0000039716 | CCTGACACTTACAACAGTCAA |
| TRC#4 | | |
| shNF1_ | TRCN0000039717 | GCTGGCAGTTTCAAACGTAAT |
| TRC#5 | | |
| shDAB2IP_ | TRCN0000001457 | GTAATGTAACTATCTCACCTA |
| TRC#1 | | |
| shDAB2IP_ | TRCN0000001458 | GACTCCAAACAGAAGATCATT |
| TRC#2 | | |
| shDAB2IP_ | TRCN0000001459 | GAGTTCATCAAAGCGCTGTAT |
| TRC#3 | | |
| shDAB2IP_ | TRCN0000001460 | CTGCAAGACTATCAACTCCTA |
| TRC#4 | | |
| shDAB2IP_ | TRCN0000001461 | GCACATCACTAACCACTACCT |
| TRC#5 | | |

shTGFβR2#1, TRCN0000000830;
shTGFβR2#2, TRCN0000010445.

The mouse Med12 expression constructs were generated by the following steps:

1), An linker containing first 89 bp of Med12 open reading frame (ORF) and multiple restriction sites was cloned into pcDNA3.1(+) vector by NheI and BamHI restriction sites and was sequence verified; The oligo sequences of the top strand for the linker is CTAGCTCGAGTCGACCATGGCGGCTTTCGGGATCTTGAGCTATGAACACCGACCC CTGAAGCGGCTGCGGCTGGGGCCTCCCGATGTGTACCCTCAG and the bottom strand is GATCCTGAGGGTACACATCGGGAGGCCCCAGCCGCAGCCGCTTCAGGGGTCGGT GTTCATAGCTCAAGATCCCGAAAGCCGCCATGGTCGACTCGAG.

2), A PCR fragment of partial Med12 (from 89 to 1777 bp) was generated using a forward primer (CAGGATCCCAAACAGAAGGAGGATGAACTGACGGCTTTGAATGTAA), a reverse primer (TGGGAGAAGACATCATGTCG) and a Med12 partial cDNA as the template (IMAGE id: 6830443); This PCR fragment was then cloned into the pcDNA3.1(+)-Med12 (first 89 bp) vector described in step 1 by BamHI and EcoRI restriction sites and was sequence verified. Note that a silence mutation (A to G) at 81 bp of Med12 ORF was introduced in the forward PCR primer to generate BamHI site in the PCR fragment.

3), An EcoRI/NotI fragment (containing from 1778 to 6573 bp of Med12 ORF) from the Med12 partial cDNA (IMAGE id: 6830443) was cloned into the pcDNA3.1 (+)-Med12 (first 1777 bp) described above by EcoRI and NotI restriction sites to generate the pcDNA3.1(+)-Med12 (full-length).

4), The XhoI/NotI fragment containing the full-length Med12 ORF from pcDNA3.1(+)-Med12 was then cloned into the retroviral expression vector pMX-IRES-blasticidine using the XhoI and NotI restriction sites.

The human SMARCE1 expression construct and the non-degradable (ND) forms of were generated by PCR amplifying SMARCE1 from H3122 cDNA using the following primers:

### Forward, GTACGAATFCCACCATGTCAAAAAGACCATCTTATGC;

Reverse, GAATAAGTGTTGCCTTGTTTTGTGCTCGAGACTG. The fragment was cloned into the retroviral expression vector pMX-IRES-blasticidine using the EcoRI and XhoI restriction sites in the multiple cloning site and sequence verified. The SMARCE1-ND that is resistant against shSMARCE1#1 was generated by site directed mutagenesis using the following primer pair:

### Forward, GCATGGAGAAAGGAGAGCCATATATGAGCATTCAGCCTG;

### Reverse, CAGGCTGAATGCTCATATATGGCTCTCCTTTCTCCATGC.

The SMARCE1-ND that is resistant against shSMARCE1#2 was generated by site directed mutagenesis using the following primer pair:

### Forward, GAAGCTGCTTTAGAGGAGGAGAGCCGACAGAGACAATCTC;

Reverse, GAGATTGTCTCTGTCGGCTCTCCTCCTCTAAAGCAGCTTC. Both SMARCE1-ND clones were sequence verified.

Retroviral expression constructs (pBabe) for KRASG 12V (#12544), MEK-DD (#15268), RALAQ75L (#19719), RALBQ72L (#19721), PIK3CAH1047R (#12524) and pCMV5BTGFbeta receptor II (#24801) were obtained from Addgene and sequence validated. The pBabe-BRAFV600E plasmid was a kind gift of Daniel Peeper. The cDNA encoding Myr-AKT was cloned into pBabe-puro and validated by sequencing. These active alleles of RAS effector pathways were also described previously (Holzel et al., 2010)

Quantitative RT-PCR (qRT-PCR)

QRT-PCR assays were carried out to measure mRNA levels of genes using 7500 Fast Real-Time PCR System. (Applied Biosystems). Total RNA was isolated using Trizol (Invitrogen) and 1 μg of total RNA was used for cDNA synthesis using superscript II reverse transcriptase (Invitrogen) and random hexamer primers (Invitrogen). Relative mRNA levels of each gene shown were normalized to the expression of the house keeping gene GAPDH. The sequences of the primers for assays using SYBR® Green master mix (Roche) are listed below (h, human: m, mouse).

| | |
|---|---|
| hGAPDH_QPCR_Forward | AGGTGAAGGTCGGAGTCAA |
| hGAPDH_QPCR_Reverse | AATGAAGGGGTCATTGATGG |
| hNF1_QPCR_Forward | TGTCAGTGCATAACCTCTTGC |
| hNF1_QPCR_Reverse | AGTGCCATCACTCTTTTCTGAAG |
| hMED12_QPCR_Forward | GCTGGTGCACATAGCCACT |
| hMED12_QPCR_Reverse | TACTCCAGCCAGCCTTACCA |
| mMed12_QPCR_Forward | TCAGGCAGTGGGATTACAATGA |
| mMed12_QPCR_Reverse | TCCAGGGCGTATTTTCTCAAAAC |
| hSMARCE1_QPCR_Forward | CGGCTTATCTGGTGGCTTT |
| hSMARCEl_QPCR_Reverse | AACAACTACAGGCTGGGAGG |
| hSMARCE1_3′UTR_QPCR_Forward | GGCTTTTGGACCATTTAGCA |
| hSMARCE1_3′UTR_QPCR_Reverse | GAGGCTTTCAGCAGTTGAGG |
| hARID1A_QPCR_Forward | CCAACAAAGGAGCCACCAC |
| hARID1A_QPCR_Reverse | TCTTGCCCATCTGATCCATT |
| hDAB2IP_QPCR_Forward | AGCGAGACTCCTTCAGCCTC |
| hDAB2IP_QPCR_Reverse | GACCGCAACCACAGCTTC |
| TGFβR2_Forward, | GCACGTTCAGAAGTCGGTTA; |
| TGFβR2_Reverse, | TCTGGTTGTCACAGGTGGAA; |
| ANGPTL4_Forward, | GGAACAGCTCCTGGCAATC; |
| ANGPTL4_Reverse, | GCACCTAGACCATGAGGTGG; |
| TAGLN_Forward, | GTCCGAACCCAGACACAAGT; |
| TAGLN_Reverse, | CTCATGCCATAGGAAGGACC; |
| CYR61_Forward, | GCTGGAATGCAACTTCGG; |
| CYR61_Reverse, | CCCGTTTTGGTAGATTCTGG; |
| CTGF_Forward, | TACCAATGACAACGCCTCCT; |
| CTGF_Reverse, | TGGAGATTTTGGGAGTACGG; |
| VIM_Forward, | CTTCAGAGAGAGGAAGCCGA; |
| VIM_Reverse, | ATTCCACTTTGCGTTCAAGG; |
| CDH2_Forward, | CCACCTTAAAATCTGCAGGC; |
| CDH2_Reverse, | GTGCATGAAGGACAGCCTCT. |

### REFERENCES

Berns, K., Hijmans, E. M., Mullenders, J., Brummelkamp, T. R., Velds, A., Heimerikx, M., Kerkhoven, R. M., Madiredjo, M., Nijkamp, W., Weigelt, B., et al. (2004). A large-scale RNAi screen in human cells identifies new components of the p53 pathway. Nature 428, 431-437.
Brummelkamp, T. R., Bernards, R., and Agami, R. (2002). A system for stable expression of short interfering RNAs in mammalian cells. Science 296, 550-553.
Brummelkamp, T. R., Fabius, A. W., Mullenders, J., Madiredjo, M., Velds, A., Kerkhoven, R. M., Bernards, R., and Beijersbergen, R. L. (2006). An shRNA barcode screen provides insight into cancer cell vulnerability to MDM2 inhibitors. Nat Chem Biol 2, 202-206.

### TGFβ Signaling is Required for Drug Resistance Caused by MED12 Loss

The studies described herein show that suppression of MED12 leads to ERK activation and thus confers what in some embodiments is a “multi targeted-drug resistance” phenotype. To gain further mechanistic insights, Applicants set out to screen a lentiviral shRNA library representing the full complement of 518 human kinases (the “kinome”, (Manning et al., 2002)) and 17 additional kinase-related genes (FIG. 36) for genes whose inhibition restores sensitivity to ALK inhibitors in MED12^{KD} cells. This “drop out” screen is the inverse of the resistance screen shown in FIG. 2A,B, as here Applicants select for shRNAs that are depleted upon drug treatment rather than enriched. H3122 cells stably expressing shMED12 were infected with the lentiviral kinome shRNA collection and cultured in the presence or absence of crizotinib for 10 days. After this, the relative abundance of shRNA vectors was determined by next generation sequencing of the bar code identifiers present in each shRNA vector (FIG. 21A). To prioritize the candidates for study, Applicants arbitrarily considered only shRNA vectors that had been sequenced at least 200 times and which were depleted at least 2.5 fold by the drug treatment. Only very few of the 3388 shRNA vectors in the library met this stringent selection criterion (FIG. 21B). Among these candidates, only one gene, transforming growth factor beta receptor II (TGFβR2), was represented by two independent shRNA vectors that met the selection criterion. This suggested that suppression of TGFβR2 synergizes with ALK inhibition in MED12KD cells. To validate this finding, Applicants infected the same MED12^{KD} H3122 cells with each of these two shTGFβR2 vectors (both of which reduced TGFβR2 levels (FIG. 21D)) and cultured these cells with or without crizotinib for two weeks. Inhibition of TGFβR2 did not significantly affect proliferation of the parental or MED12KD cells in the absence of crizotinib (FIG. 21C). In contrast, suppression of TGFβR2 in combination with ALK inhibitor caused a marked inhibition of proliferation only in MED12KD cells (FIG. 21C). These findings indicate that suppression of TGFβR2 re-sensitizes the MED12^{KD} cells to ALK inhibitors and suggest that TGFβ signaling is required for the drug resistance driven by MED12 loss.

### TGFβ Activation is Sufficient to Confer Resistance to Multiple Targeted Drugs in Different Cancer Types

Next, Applicants asked whether activation of TGFβ signaling alone is sufficient to cause resistance to the cancer drugs studied above. In the absence of exogenous TGFβ, proliferation of the H3122 cells was greatly inhibited by crizotinib. In contrast, cells treated with TGFβ in combination with crizotinib continued to proliferate in a TGFβ-dosage dependent manner (FIG. 4A). These data indicate that TGFβ activation, similar to suppression of MED12, is sufficient to confer resistant to ALK inhibitors in EML4-ALK positive NSCLCs. Interestingly, H3122 cells treated with recombinant TGFβ had a similar large and flat cell morphology as MED12KD cells, which was not seen in parental cells (FIG. 26A). Similar morphological observations were seen in other cell types (FIG. 26B and data not shown).

Recombinant TGFβ treatment also conferred resistance to EGFR inhibitors in PC9 and H3255 NSCLC cells (FIG. 24B and data not shown). Similarly, treatment of TGFβ resulted in a dosage dependent resistance to AZD6244 and PLX4032 in SK-CO-1 CRC cells and A375 melanoma cells (FIG. 24C, D). In some cells, such as A375 and Huh-7 cells, (FIG. 24D and data not shown), recombinant TGFβ treatment alone resulted in growth inhibition, but clearly became beneficial for proliferation when cells were cultured in the presence of targeted cancer drugs, mimicking the effects of MED12 knock down in the same cells (FIG. 17C, G). Collectively, these results demonstrate that activation of TGFβ signaling is sufficient to confer resistance to multiple targeted cancer drugs in the same cancer types in which MED12^{KD} also confers drug resistance.

### MED12 Loss Activates TGFβ Signaling by Elevating TGFβR2 Protein Levels

The fact that TGFβ signaling is required for the drug resistance driven by MED12 suppression and that activation of TGFβ signaling phenocopies MED12^{KD} in mediating drug resistance suggested that MED12 can act as a suppressor of TGFβ signaling. Applicants explored this possibility by studying differential gene expression by unbiased transcriptome sequencing analysis using next generation sequencing (RNA-Seq) for the same panel of cells lines tested above (H3122, PC9, SK-CO-1, A375 and Huh-7), for both the parental cells and multiple MED12KD derivatives thereof. The genes deregulated by MED12^{KD} (>2 fold) in at least three out of five cell lines used are listed in FIG. 37 and are referred to as MED12^{KD} signature genes henceforth (237 genes up—and 22 genes downregulated). Strikingly, many of these genes are bona fide TGFβ targets. To confirm these observations, Applicants first examined mRNA expression levels of a panel of TGFβ target genes, including ANGPTL4, TAGLN, CYR61, CTGF, SERPINE1 and CDKN1A in both H3122 and PC9 cells by qRT-PCR (FIG. 29A to 29D and data not shown). In agreement with Applicants' RNA-Seq data, all of these TGFβ target genes were significantly induced upon MED12^{KD} in these NSCLC cells. Applicants also observed induction of these TGFβ target genes upon MED12^{KD} in many cell lines of other tumor types, including melanoma A375 and SK-MEL-28, CRC SK-CO-1 and SW1417 and HCC Huh-7 (FIG. 30A to 30D and data not shown). It is well-established that TGFβ induces an epithelial-mesenchymal transition (EMT), leading to the induction of several mesenchymal markers such as Vimentin (VIM) and N-cadherin (CDH2) (Thiery et al., 2009). Importantly, MED12^{KD} also caused expression of the mesenchymal markers VIM and CDH2, indicating that an EMT-like process is initiated in MED12^{KD} cells (FIG. 29E-F and FIG. 30E-F). Accordingly, the protein products of these mesenchymal-specific genes such as Vimentin and N-cadherin were also detected in MED12^{KD} cells by Western blotting (FIG. 30I and data not shown). Expression of the epithelial marker E-cadherin (CDH1) was not lost in MED12^{KD} cells (data not shown), suggesting that MED12^{KD} induces a partial EMT. Together, these unbiased gene expression studies support the notion that MED12 is a suppressor of TGFβ signaling in a wide range of cancer types and that its loss activates TGFβ signaling.

To further elucidate the molecular mechanism by which MED12 suppresses TGFβ signaling, Applicants studied the effect of knockdown of MED12 on expression and activation of key components of the TGFβ signaling pathway. Strikingly, Applicants found that suppression of MED12 resulted in a strong induction of TGFβR2 protein levels in H3122 and PC9 cells (FIG. 29G, H). Consistently, SMAD2, the key mediator for TGFβ target gene activation, was activated as indicated by a strong increase in SMAD2 phosphorylation upon MED12 knockdown. Similar results were also obtained in A375 melanoma, in SK-CO-1 CRC cells and other cancer cell lines, indicating that this interplay between MED12KD and TGFβ signaling is conserved across different tumor types (FIG. 30H-I and data not shown).

Since MED12 is part of the MEDIATOR transcriptional complex that functions in the nucleus, Applicants assumed that MED12 would act on TGFβR2 through a transcriptional step. However, there was only a marginal increase of TGFβR2 mRNA upon MED12 knockdown (FIG. 30G), suggesting that MED12 suppresses TGFβR2 in a post-transcriptional manner. To investigate this, Applicants first determined the subcellular localization of MED12. Applicants carried out nuclear and cytoplasmic fractionation of PC9 cells expressing control vector or shMED12, followed by western blotting (FIG. 29I). Lamin A/C and SP1 were used as marker controls for nuclear fractions, while α-TUBULIN and HSP90 were used as controls for cytoplasmic fractions. Abundant nuclear MED12 was detected, in agreement with its known function in a transcriptional complex. Unexpectedly, a significant quantity of MED12 was also present in the cytoplasmic fraction. Applicants confirmed that the cytoplasmic MED12 signal was genuine as it was greatly reduced in the lysate from MED12KD cells. Cytoplasmic MED12 was also seen in H3122 cells (FIG. 30J). Interestingly, no significant cytoplasmic CDK8 was detected, another subunit of the MEDIATOR kinase module with which MED12 is known to associate closely. This suggested that cytoplasmic MED12 might have a second function, independent of its role in the MEDIATOR complex.

The observation of the cytoplasmic localization of MED12 prompted Applicants to examine a potential physical interaction between MED12 and TGFβR2. Since low expression of endogenous TGFβRs in most cell types hinders the study of physical interaction with TGFβRs, Applicants performed co-immunoprecipitation experiments using Phoenix cells cotransfected with TGFβR2 and MED12. As indicated in FIG. 29J, TGFβR2 coimmunoprecipitated with MED12 and conversely MED12 co-immunoprecipitated with TGFβR2, indicating that MED12 interacts physically with TGFβR2. Thus, in certain embodiments, MED12 is a critical suppressor of TGFβ signaling by negatively regulating TGFβR2 and this effect is mediated in certain embodiments by a novel cytoplasmic function of MED12 in complex with TGFβR2. Hence, without being bound to theory, this finding provides an explanation why MED12 suppression leads to activation of TGFβ signaling.

### a MED12KD Gene Signature has Features of EMT and is Both Prognostic and Predictive

As described above, MED12 suppression leads to activation of TGFβ signaling and expression of mesenchymal markers, suggestive of a partial EMT-like process. Recently, EMT has been identified as a program in human CRC that correlates with poor prognosis (Loboda et al., 2011). Applicants therefore asked whether MED12^{KD} indeed induces an EMT-like process and whether the processes induced by MED12KD are likewise associated with poor survival in CRC.

Applicants first compared the 237 genes that were upregulated in the MED12^{KD} signature (as described herein; FIG. 37) to the 229 genes upregulated in a more general EMT signature (see FIG. 38). Applicants found a significant overlap of 31 genes in both signatures (p=8.9*10-23; FIG. 33A and FIG. 39). This result further supports the notion that MED12 loss initiates a partial EMT. There was no overlap between the 22 genes downregulated in the MED12KD signature and the genes downregulated in the EMT signature, most likely due to the small number of genes. Next, Applicants asked whether genes that are deregulated after MED12 knockdown predict survival in CRC. Hierarchical clustering of a set of 231 CRC tumor samples using the MED12^{KD} signature genes led to the identification of two subsets of tumors having significantly different disease-specific survival (FIG. 33B). These results indicate that the processes induced by MED12^{KD} result in a poor survival in CRC patients.

To further substantiate Applicants' finding that MED12 suppression confers resistance to cancer drugs targeting the MEK-ERK pathway downstream of RTKs, Applicants asked if the MED12^{KD} signature could predict responses to MEK inhibitors in a large and heterogeneous panel of cancer cell lines of different tissue types. Since MEK inhibitors are currently being evaluated for the treatment of tumors having activating mutations in RAS or BRAF, Applicants focused their studies on 152 tumor cell lines harboring either RAS or BRAF mutations for whom the IC50 values of four different MEK inhibitors and gene expression patterns have been determined (FIG. 41). Of the 237 genes that were up-regulated by MED12D as identified by RNA-Seq, Applicants could read the expression levels for 170 genes in these 152 cell lines (FIG. 40). Applicants found that high expression of these 170 genes is significantly associated with higher IC50s for all four MEK inhibitors in these cell lines (AZD6244, p=0.009; CI-1040, p=0.004; PD-0325901, p=0.007; RDEA119, p=0.013; FIG. 33C and FIG. 40). The analysis of one of these genes, ZBED2, is shown as an example in FIG. 34). Thus, the group of genes that is upregulated following MED12^{KD} can predict response to MEK inhibitors in a very heterogeneous panel of cancer cell lines, consistent with the notion that MED12 acts independent of cellular context to influence cancer drug responses (FIG. 33C).

### TGFβR Inhibitor and TKIs Synergize to Suppress Proliferation of MED12KD NSCLC Cells

Applicants have demonstrated that TGFβ activation by either MED12 loss or recombinant TGFβ stimulation confers resistance to multiple targeted cancer drugs in a range of cancer types. It is therefore of potential clinical relevance to explore new treatment strategies to target drug resistant tumors having acquired elevated TGFβ signaling. Since inhibition of TGFβR2 by RNAi re-sensitized MED12^{KD} NSCLC cells to TKIs (FIG. 21 and data not shown), Applicants reasoned that TGFβR inhibitors would synergize with TKIs to inhibit MED12^{KD} NSCLC cells.

To test this concept; Applicants cultured control or MED12^{KD} H3122 cells in the absence and the presence of crizotinib, the TGFβR inhibitor LY2157299 or the combination of crizotinib and LY2157299 (FIG. 35A). LY2157299 is a small molecule inhibitor targeting both TGFβR1 and TGFβR2, and is currently being evaluated in clinical trials for the treatment of several cancer types. Consistent with Applicants' previous data, crizotinib alone potently inhibited the proliferation of the control, but not of the MED12^{KD} cells. LY2157299 monotherapy had little effect on all cells. However, strong synergy was seen when crizotinib was combined with LY2157299, consistent with the notion derived from the RNAi experiment that TGFβR2 inhibition restored the sensitivity of MED12^{KD} cells to crizotinib. Importantly, the same synergistic response was also obtained when LY2157299 was combined with gefitinib to suppress proliferation of MED12KD PC9 cells (FIG. 35B). Thus, in certain embodiments, the combination of TGFβR inhibitors and TKIs is a strategy for treating tumors with elevated TGFβ signaling.

### Experimental Procedures

Pooled “Dropout” shRNA Screen

A Kinome shRNA library targeting the full complement of 518 human kinases and 17 kinaserelated genes was constructed from the TRC human genome-wide shRNA collection (TRCHs1.0). The Kinome library was used to generate pools of lentiviral shRNA to infect H3122 cells stably expressing shMED12. Cells were cultured in the presence or absence of crizotinib. Massive parallel sequencing was applied to determine the abundance of shRNA in cells. shRNAs prioritized for further analysis were selected by the fold of depletion by crizotinib treatment.

### Long-Term Cell Proliferation Assays

Cells were seeded into 6-well plates (2-5×10⁴ cells/well) and cultured both in the absence and presence of drugs as indicated. More details are described in Huang et al., 2009 (Huang et al., 2009). All knockdown and overexpression experiments were done by retroviral or lentiviral infection. All relevant assays were performed independently at least three times.

### Gene Expression and Statistical Analysis

Transcriptome sequencing analysis of cell lines were performed using RNA-Seq. To rule out “off-target” effects, Applicants considered genes that are significantly deregulated in the same direction by two independent shMED12 vectors. The MED12KD gene signature was then assembled containing genes that were more than 2 folds up- or downregulated upon MED12 knock-down in at least three out of five cell lines. This signature was employed to hierarchically cluster a dataset consisting of gene expression data for 231 which CRC tumor samples. Differences in disease specific survival were determined using the Kaplan-Meier statistics.

### EMT Signature

An EMT signature was created by combining EMT expression signatures published by Taube et al. (Taube et al., 2010) and Loboda et al. (Loboda et al., 2011), and from the SABiosciences EMT PCR array (SABiosciences, Frederick, Md.). All genes were annotated as down- or upregulated during EMT according to the source. Genes with annotation of conflicting expression changes in several sources were excluded. All gene symbols were translated to probe set identifiers.

### COSMIC Cell Line Panel Analysis

Drug response data (IC50 values) and gene expression levels were obtained from COSMIC (Forbes et al., 2010) for 152 cell lines that have activating mutations in RAS or BRAF. The IC50 values were classified as sensitive or resistant and gene expression levels were classified as normal, up- or downregulated. For each pair of a gene and a MEK inhibitor an overlap enrichment test was applied to evaluate if significantly many cell lines were both upregulated for the gene and resistant to the MEK inhibitor. The number of significant associations within in the MED12 signature gene set was counted and compared to 100,000 randomly drawn sets of the same size and variance distribution to evaluate the significance of the MED12 signature.

### Nuclear and Cytoplasmic Fractionation

Subcellular fractionation experiments were performed according manufacture protocol using the NE-PER Nuclear and Cytoplasmic Extraction Kit (78835) purchased from Thermo Scientific.

shRNA “Dropout” Screen with a Custom TRC Kinome Library

Lentiviral plasmids (pLKO.1) encoding shRNA that target kinome candidates were listed in FIG. 36. The kinome library consists of 7 plasmids pools (TK1-TK7).

Lentiviral supernatants were generated as described at http://www(dot)broadinstitute(dot)org/rnai/public/resources/protocols. H3122 cells stably expressing shMED12#3 were infected separately by the 7 virus pools (Multiplicity Of Infection of 1). Cells were then pooled and plated at 300,000 cells per 15 cm dish in absence or presence of 300 nM crizotinib (5 dishes for each condition) and the medium was refreshed twice per week for 10 days. Genomic DNA was isolated as described (Brummelkamp et al., 2006). shRNA inserts were retrieved from 8 ug genomic DNA by PCR amplification (PCR1 and PCR2, see below for primer information) using the following conditions: (1) 98° C., 30 s; (2) 98° C., 10 s; (3) 60° C., 20 s; (4) 72° C., 1 min; (5) to step 2, 15 cycles; (6) 72° C., 5 min; (7) 4° C. Indexes and adaptors for deep sequencing (Illumina) were incorporated into PCR primers. 2.5 ul PCR1 products were used as templates for PCR2 reaction. PCR products were purified using Qiagen PCR purification Kit according to the manufacturer manual. Sample quantification is performed by BioAnalyzer to ensure samples generated at different conditions were pooled at the same molar ratio before analyzed by Illumina genome analyzer.

shRNA stem sequence was segregated from each sequencing reads and aligned to TRC library. The matched reads were counted and the counts were transformed to abundance that was assigned to the corresponding shRNA.

Primers used are as follows:

PCR1_Untreated replicate#1_Forward,
ACACTCTTTCCCTACACGACGCTCTTCCGATCTCTGATCCTTGTGGAAAG
GACGAAACACCGG;
PCR1_Untreated replicate#2_Forward,
ACACTCTTTCCCTACACGACGCTCTTCCGATCTAAGCTACTTGTGGAAAG
GACGAAACACCGG;
PCR1_PLX treated replicate#1_Forward,
ACACTCTTTCCCTACACGACGCTCTTCCGATCTGTAGCCCTTGTGGAAAG
GACGAAACACCGG;
PCR1_PLX treated replicate#1_Forward,
ACACTCTTTCCCTACACGACGCTCTTCCGATCTTACAAGCTTGTGGAAAG
GACGAAACACCGG;
PCR1_Reverse (P7_pLKO1_r),
CAAGCAGAAGACGGCATACGAGATTTCTTTCCCCTGCACTGTACCC;
PCR2_Forward,
AATGATACGGCGACCACCGAGATCTACACTCTTTCCCTACACGACGCTCT
TCCGATCT:
PCR2_Reverse (P5_IlluSeq),
CAAGCAGAAGACGGCATACGAGAT.

### RNA-Seq Gene Expression Analysis

Total mRNA of each sample was converted into a library of template molecules suitable for subsequent cluster generation using the reagents provided in the Illumina® TruSeq™ RNA Sample Preparation Kit, following the manufacture protocol. Sequence reads were generated using Illumina HiSeq 2000 with TruSeq™ v3 reagent kits and software. The reads (between 20-45 million 50 bp paired-end reads per sample) were mapped to the human reference genome (build 37) using TopHat (v. 1.3.1, (Trapnell et al., 2009)), which allows to span exon-exon splice junctions. The open-source tool HTSeq-count (v. 0.5.3p3), available from EMBL, was then used to generate a list of the total number of uniquely mapped reads (between 16-33 million pairs of reads per sample) for each gene that is present in the provided Gene Transfer Format (GTF) file.

In order to determine which genes are differentially expressed between samples, the R package DEGseq (Wang et al., 2010) was used, which takes the output of HTSeq-count as input. The method used to identify differentially expressed genes is the MA-plot-based method with technical Replicates (MATR), which makes use of the presence of technical replicates. The genes that have no expression for all samples in the comparison were discarded from the dataset. The expression levels of all remaining genes in the dataset were added with 1 in order to avoid negative values after log 2 transformation. Normalization for the number of reads is performed within this method and the cut off for differentially expressed genes is based on a p-value of 0.05.

### Gene Expression Statistical Analysis

Gene expression datasets GSE14333 (Jorissen et al., 2009), GSE17536 and GSE17537 (Smith et al., 2010) were downloaded from the Gene Expression Omnibus (Barrett et al., 2011).

Duplicated samples in GSE14333 and GSE17536 were removed from GSE14333 resulting in a final dataset comprising 389 tumor samples. Expression data were first normalized together using the RMA method as implemented in the affy package (Gautier et al., 2004) for R/Bioconductor (Gentleman et al., 2004) and then mean-centered separately for each dataset. The hclust method was employed for hierarchically clustering the samples based on MED12KD and Pearson correlation distance. The survival and Design packages were used for performing a Kaplan-Meier survival time analysis and plotting survival curves, respectively.

### COSMIC Cell Line Panel Analysis

The predictive value of the MED12 knockdown signature was assessed using the Catalogue Of Somatic Mutations In Cancer (COSMIC), which is part of the Cancer Genome Project (CGP) (Forbes et al., 2010). From COSMIC Applicants collected the IC50 values of four MEK inhibitors (AZD6244, CI-1040, PD-0325901 and RDEA119) for 152 cell lines that have a mutation in KRAS, HRAS, NRAS and/or BRAF. For these cell lines Applicants also obtained gene expression levels for 11354 genes from COSMIC.

The IC50 values across the 152 cell lines for each MEK inhibitor were discretized into “sensitive” and “resistant” using a simple discretization strategy. Briefly, if the distribution of IC50 values was not unimodal (using Hartigan's dip test (Hartigan and Hartigan, 1985), p<0.05), a two component Gaussian mixture model was used to assign the cell lines to the sensitive or resistant category. Otherwise, an outlier detection strategy was used to call the cell lines that are far to the left of the bulk of the data (i.e., low IC50 values) as sensitive and the others as resistant. Overall, about 18% of the cell lines were called sensitive for each of the MEK inhibitors.

The same strategy was used to discretize the expression levels of each gene into “downregulated”, “normal”, and “upregulated.” In this case, either a two or three component mixture model was used for multimodal distributions (using the BIC to choose the number of components), and for unimodal distributions the outlier scheme called cell lines to the right of the bulk (i.e. high expression levels) as upregulated and those to the left (i.e. low expression levels) as down-regulated.

Next, for each pairing of a gene and a MEK inhibitor a simple enrichment test (i.e. hypergeometric test) was applied to evaluate if significantly many cell lines were both upregulated for the gene and resistant to the MEK inhibitor. For the four MEK inhibitors, AZD6244, CI-1040, PD-0325901 and RDEA119, Applicants respectively detected 474, 807, 856 and 681 genes at p<0.05.

Applicants evaluated whether there was an overrepresentation of the MED12 signature genes in these sets of genes. Of the 237 genes upregulated after MED12 knockdown, 170 are part of the gene expression set of COSMIC. Of the 22 genes downregulated after MED12 knockdown, only 12 are present in the gene expression set. Because the latter set is very small, Applicants decided to focus only on the set of 170 upregulated genes. In these 170 genes, and the four MEK inhibitors, AZD6244, CI-1040, PD-0325901 and RDEA119, Applicants detected 22, 36, 35, and 26 genes at p<0.05, respectively. Seven genes were found in all of the four groups. The association of gene expression with response to AZD6244 for one of these genes, ZBED2, is depicted in FIG. 34.

In order to determine the statistical significance of the number of genes in the MED12 signature whose gene expression was found to be associated with each of the inhibitors, Applicants compared these numbers to what would be expected under the null hypothesis. More specifically, Applicants randomly drew 100,000 sets of 170 genes with the same distribution of expression variance across the dataset as the 170 MED12 upregulated signature genes. Applicants computed a permutation test p-value, which indicates the fraction of times (out of 100,000) that the randomly drawn gene set showed more significantly associated genes than the 170 MED12 signature genes. These p-values are 0.009, 0.004, 0.007 and 0.013 for AZD6244, CI-1040, PD-0325901 and RDEA119, respectively. These numbers are found in FIG. 33C and in the main text.

Applicants observed that the variance of genes in the MED12 signature was higher than the average for the complete expression dataset. Applicants focused on random gene sets with the same variance distribution, since genes with no or low variance across the dataset can never be significantly associated with the varying IC50 values, and should therefore not be part of the random gene sets.

Having thus described in detail embodiments of the present invention, it is to be understood that the invention defined by the above paragraphs is not to be limited to particular details set forth in the above description as many apparent variations thereof are possible without departing from the spirit or scope of the present invention.

Each patent, patent application, and publication cited or described in the present application is hereby incorporated by reference in its entirety as if each individual patent, patent application, or publication was specifically and individually indicated to be incorporated by reference.

1. A method of predicting resistance to anticancer treatment in a patient in need thereof, comprising:
(a) measuring expression levels of one or more SWI/SNF complex nucleic acid and/or proteins in the patient; and
(b) comparing the expression levels of the one or more SWI/SNF complex nucleic acid and/or proteins in (a) with the expression levels of one or more reference SWI/SNF complex nucleic acid and/or proteins, wherein the one or more reference SWI/SNF complex nucleic acid and/or proteins are from a control sample,
wherein a reduction in the expression of the one or more SWI/SNF complex nucleic acid and/or proteins in comparison to the one or more reference SWI/SNF complex nucleic acid and/or proteins is indicative of resistance to anticancer treatment in the patient; and/or
(c) isolating nucleic acid from the patient, wherein the nucleic acid comprises one or more SWI/SNF complex DNA and/or RNA; and
(d) analyzing the nucleic acid of (c) for the presence of one or more inactivating mutations in the SWI/SNF complex DNA and/or RNA in comparison to one or more reference SWI/SNF complex DNA and/or RNA,
wherein the presence of one or more inactivating mutations in the one or more SWI/SNF complex DNA and/or RNA analyzed in (d) is indicative of resistance to anticancer treatment in the patient; and/or
(e) isolating protein from the patient, wherein the protein comprises one or more SWI/SNF complex proteins;
(f) analyzing the activity of the one or more SWI/SNF complex proteins in (e); and
(g) comparing the activity of the one or more SWI/SNF complex proteins in (f) with the activity of one or more reference SWI/SNF complex proteins,
wherein a difference in activity of the one or more SWI/SNF complex proteins from (f) in comparison to the one or more SWI/SNF complex reference proteins in (g) is indicative of resistance to anticancer treatment in the patient.

2.-19. (canceled)

20. The method of claim 1, wherein the resistance to anticancer treatment is resistance to treatment with a receptor tyrosine kinase inhibitor.

21. (canceled)

22. The method of claim 1, wherein the resistance to anticancer treatment is resistance to treatment with an inhibitor of ERK activation.

23.-25. (canceled)

26. The method of claim 1, wherein the SWI/SNF complex nucleic acid and/or protein is selected from the group consisting of: ARID1A, ARID1B, ARID2, SMARCA2, SMARCA4, PBRM1, SMARCC2, SMARCC1, SMARCD1, SMARCD2, SMARCD3, ACTL6A, ACTL6B, and SMARCB1.

27. The method of claim 26, wherein the SWI/SNF complex nucleic acid and/or protein is ARID1A.

28.-31. (canceled)

32. The method of claim 1, wherein the patient has lung cancer.

33. (canceled)

34. The method of claim 1, wherein the patient has melanoma.

35. The method of claim 1, wherein the nucleic acid expression levels in (a) are measured using a microarray comprising a plurality of polynucleotide probes each complementary and hybridizable to a sequence in a different gene that is a SWI/SNF complex gene that is a marker for resistance to anticancer treatment in a patient that has cancer and wherein analyzing the nucleic acid in (d) comprises sequencing the nucleic acid or subjecting the nucleic acid to a method selected from the group consisting of: MLPA, CGH, and FISH.

36.-77. (canceled)

78. The method of claim 1, wherein the resistance to anticancer treatment is resistance to treatment with a B-RAF inhibitor or a MEK inhibitor.

79.-94. (canceled)

95. The method of claim 102, wherein the resistance to anticancer treatment is resistance to one or more inhibitors of ERK activation in the patient, and wherein the method further comprises treating resistance to one or more inhibitors of ERK activation by administering to the patient at least one inhibitor of the TGF-beta pathway in combination with the one or more inhibitors of ERK activation.

96.-100. (canceled)

101. The method of claim 1, wherein the resistance to anticancer treatment is resistance to one or more inhibitors of ERK activation in the patient, and wherein the method further comprises treating resistance to one or more inhibitors of ERK activation by administering to the patient at least one inhibitor of the TGF-beta pathway in combination with the one or more inhibitors of ERK activation.

102. A method of predicting resistance to anticancer treatment in a patient in need thereof comprising:
(a) measuring expression levels of one or more TGFβ pathway nucleic acid and/or proteins in the patient; and
(b) comparing the expression levels of the one or more TGFβ pathway nucleic acid and/or proteins in (a) with the expression levels of one or more reference TGFβ pathway nucleic acid and/or proteins, wherein the one or more reference TGFβ pathway nucleic acid and/or proteins are from a control sample,
wherein an increase in the expression of the one or more TGFβ pathway nucleic acid and/or proteins in comparison to the one or more reference TGFβ pathway nucleic acid and/or proteins is indicative of resistance to anticancer treatment in the patient; and/or
(c) isolating nucleic acid from the patient, wherein the nucleic acid comprises one or more TGFβ pathway DNA and/or RNA; and
(d) analyzing the nucleic acid of (c) for the presence of one or more activating mutations in the TGFβ pathway complex DNA and/or RNA in comparison to one or more reference TGFβ pathway complex DNA and/or RNA,
wherein the presence of one or more activating mutations in the one or more TGFβ pathway DNA and/or RNA analyzed in (d) is indicative of resistance to anticancer treatment in the patient; and/or
(e) isolating protein from the patient, wherein the protein comprises one or more TGFβ pathway proteins;
(f) analyzing the activity of the one or more TGFβ pathway proteins in (e); and
(g) comparing the activity of the one or more TGFβ pathway proteins in (f) with the activity of one or more reference TGFβ pathway proteins,
wherein a difference in activity of the one or more TGFβ pathway proteins from (f) in comparison to the one or more TGFβ pathway reference proteins in (g) is indicative of resistance to anticancer treatment in the patient.

103.-108. (canceled)

109. The method of claim 102, wherein the TGFβ pathway nucleic acid is a TGFβ pathway target gene selected from the group consisting of: ALOX5AP, COL5A1, TAGLN, ANGPTL4, LGALS1, IL11, LBH, and COL4A1.

110.-126. (canceled)

127. A method of predicting resistance to anticancer treatment in a patient in need thereof, comprising:
(a) measuring expression levels of one or more MED12KD signature nucleic acid and/or proteins in one or more cancer cells of the patient; and
(b) comparing the expression levels of the one or more MED12KD signature nucleic acid and/or proteins in (a) with the expression levels of one or more positive reference MED12KD signature nucleic acid and/or proteins, wherein if expression of the one or more MED12KD signature nucleic acid and/or proteins in (a) is similar to the one or more positive reference MED12KD signature nucleic acid and/or proteins, then resistance to anticancer treatment is indicated in the patient; and/or
(c) comparing the expression levels of the one or more MED12KD signature nucleic acid and/or proteins in (a) with the expression levels of one or more negative reference MED12KD signature nucleic acid and/or proteins, wherein if expression of the one or more MED12KD signature nucleic acid and/or proteins in (a) is greater or lesser than the expression of the one or more negative reference MED12KD signature nucleic acid and/or proteins, then resistance to anticancer treatment is indicated in the patient.

128.-137. (canceled)

138. The method of claim 127, wherein the one or more MED12KD signature nucleic acids are upregulated nucleic acids.

139. The method of claim 138, wherein the upregulated nucleic acids are selected from the upregulated nucleic acids presented in FIG. 37, FIG. 39, or FIG. 40.

140. (canceled)

141. (canceled)

142. The method of claim 127, wherein the one or more MED12KD signature nucleic acids are downregulated nucleic acids.

143. The method of claim 142, wherein the downregulated nucleic acids are selected from the downregulated nucleic acids presented in FIG. 37, FIG. 39, FIG. 40.

144. (canceled)

145. (canceled)

146. The method of claim 127, wherein the resistance to anticancer treatment is resistance to treatment with a MEK inhibitor or a B-RAF inhibitor.

147.-150. (canceled)

151. The method of claim 127, further comprising:
(d) comparing the expression levels of the one or more MED12KD signature nucleic acid and/or proteins in (a) with the expression levels of (i) one or more MED12 KD signature nucleic acid and/or proteins from cells known to be resistant to said anticancer treatment and (ii) one or more MED12KD signature nucleic acid and/or proteins from cells known to be sensitive to said anticancer treatment,
wherein the cancer cells of the patient are considered to be resistant if the difference in expression levels between the cells in (a) and the cells in (i) is smaller than the difference in expression levels between the cells in (a) and the cells in (ii); and
wherein the cancer cells of the patient are considered to be sensitive if the difference in expression levels between the cells in (a) and the cells in (i) is greater than the difference in expression levels between the cells in (a) and the cells in (ii); and/or
(e) comparing the expression levels of the one or more MED12KD signature nucleic acid and/or proteins in (a) with the average expression levels of (i) one or more MED12KD signature nucleic acid and/or proteins taken from two or more cell samples,
wherein the cancer cells of the patient are considered to be resistant if the difference in expression levels of the one or more MED12KD signature nucleic acid and/or proteins between the cells in (a) and the average expression levels of the one or more MED12KD signature nucleic acid and/or proteins in (i) is greater than a factor 1.2.

152.-154. (canceled)
